(19) United States
(12) Patent Application Publication
(10) Pub. No.: US 2010/0029682 A1
(43) Pub. Date: Feb. 4, 2010

(54) NOVEL CHROMENE AND THIOCHROMENE CARBOXAMIDE DERIVATIVES, METHODS FOR PREPARING SAME AND THERAPEUTIC APPLICATIONS OF SAME

(76) Inventors: Pierre Sokoloff, Belleserre (FR); Thierry Imbert, Viviers Les Montagnes (FR); Laurent Vergnes, Cambounet-Sur-Le-Sor (FR); Florence Cuisiat, Castres (FR)

Correspondence Address: BIRCH STEWART KOLASCH & BIRCH, PO BOX 747, FALLS CHURCH, VA, 22040-0747, US

(21) Appl. No.: 12/374,088

(22) PCT Filed: Jul. 20, 2007
(86) PCT No.: PCT/EP2007/057511

§ 371 Date: Jan. 16, 2009

(30) Foreign Application Priority Data
Jul. 21, 2006 (FR) 0606682

(51) Int. Cl. A61K 31/497; C07D 409/12; C07D 409/14; C07D 413/14

(52) U.S. Cl. 514/254.02; 544/376; 514/254.11; 544/368

(57) ABSTRACT

The present invention relates to novel chromene or thiochromene carboxamide derivatives, the preparation of same, pharmaceutical compositions of same and the use of same as dopamine D3 ligands as a medicament for central nervous system disorders.

The present invention relates to chromene and thiochromene carboxamide derivatives, methods for preparing same, pharmaceutical compositions containing same and therapeutic applications of same as dopamine D3 receptor (DRD3) agonists, partial agonists or antagonists for the treatment of various neurological and psychiatric conditions.

Schizophrenia is a term used to describe a group of pathologies of unknown origin which affects roughly 1% of the general population. This pathology is characterized by a variety of symptoms, classified as positive symptoms (hallucinations, delirium, disorganized thoughts) and negative symptoms (social withdrawal and emotional dulling), with onset during adolescence or young adulthood and which can persist in chronic form with intensified episodes for many years.

Patients afflicted with schizophrenia can be treated with medicaments called neuroleptics, also known as antipsychotics. The therapeutic effect of antipsychotics is generally understood to result from blockage in the brain of the receptors for the neurotransmitter dopamine. There are five known subtypes of dopamine receptors, called D1, D2, D3, D4 and D5 (Sokoloff et al., Novel dopamine receptor subtypes as targets for antipsychotic drugs. Annals New-York Academy of Sciences 1995, 757, 278); conventional antipsychotics are D2 and D3 receptor antagonists. However, antipsychotics are frequently responsible for undesirable extrapyramidal side effects (EPS) and for abnormal movements called tardive dyskinesia, which are attributed to blockage of D2 receptors in the striatal region of the brain. Receptor D3 (DRD3) blockage was suggested as being responsible for the therapeutic effects of antipsychotics (Schwartz, J. C. Eur. Neuropsychopharmacol. 2003, 13 (suppl. 4): S 166). Thus, pharmacological agents that selectively modulate DRD3 functioning are regarded as effective antipsychotics free of neurological side effects (international patent WO 91/15513).

Selective modulation of DRD3 can be obtained with molecules that bind selectively with DRD3 and that act either as agonists, antagonists, or partial agonists. Antipsychotic activity resulting from the modulation of DRD3 functioning can be predicted in animals by using mouse models of schizophrenia (Leriche, L. Neuropharmacology 2003, 45, 174). Furthermore, it has been shown that selective blockage of DRD3, but not concomitant blockage of DRD2 and DRD3, increases extracellular levels of dopamine and acetylcholine, another neurotransmitter, in the prefrontal cortex (Lacroix, L. P. Neuropsychophamacol. 2003, 28, 839). Dopamine and acetylcholine in this region of the brain are essential for cognitive functioning. Consequently, it is believed that selective DRD3 antagonists can improve cognition, which is altered in schizophrenia as well as in neurodegenerative pathologies such as Alzheimer's disease.

Depression is a common mood pathology, characterized by feelings of intense sadness, pessimistic thoughts and self depreciation, often accompanied by loss of energy, enthusiasm and libido. The inability to experience pleasure from normally pleasurable life events, also known as anhedonia, is also regarded as a common symptom of depression. An important role in pleasure and motivation has been attributed to dopaminergic neurons projecting into the nucleus accumbens region of the brain (Koob G. F. Sem. Neurosci. 1992, 4, 139; Salamone J. D. Behav. Brain Res. 1994, 61, 117). Consequently, such neurons have been implicated in the neurobiology of depression, in particular in anhedonia, and in the therapeutic effects of certain antidepressant medicaments (Kapur S. and Mann J. Biol. Psychiatry 1992, 32, 1-17; Willner P. Int. Clin. Psychopharmacol. 1997, 12, S7-S14). It has been shown more recently that various antidepressant treatments selectively increase the expression of DRD3 in the nucleus accumbens (Lammers C. H. Mol. Psychiatry. 2000, 5, 378), suggesting that increasing DRD3 functioning could be a new mode of antidepressant treatment. An increase in DRD3 D3 receptor functioning can be achieved with DRD3 aqonists or partial agonists, resulting in an effective treatment for depression.

Dependency on or addiction to drugs or other addictive substances is a chronic, recurring pathology in which risky, drug seeking behaviors and compulsive drug-taking behaviors persist in spite of the negative consequences perceived by the patient (Deroche-Gamonet V. Science 2004, 305, 1014; Vanderschuren L. J. Science 2004, 305, 1017). The withdrawal phenomenon, which appears when addicts abstain from addictive substances, can be triggered or exacerbated by environmental stimuli, which acquire a motivational power due to the fact that they have been associated repeatedly with the effects of a drug, both in man (Childress A. R. Am. J. Psychiatry 1999, 156, 11; Robinson T. E. Brain Research Reviews 1993, 18, 247) and in animals (Goldberg S. R. NIDA Res. Monogr. 1981, 37, 241; Arroyo M. Psychopharmacology 1999, 140, 331). In animals, highly selective DRD3 agonists or partial antagonists specifically reduce responses to stimuli associated with cocaine (Pilla M. Nature, 1999, 400, 371; Le Foll, B. Eur. J. Neurosci. 2002, 15, 2016; Vorel S. R. J. Neurosci. 2002, 22, 9595), with an opiate (Frances H. Neuroreport 2004, 15, 2245) or with nicotine ((Le Foll B. Mol. Psychiatry. 2003, 8, 225), while having no influence on the primary effects of the drugs. Brain DRD3 density is abnormally high in cocaine addicts (Staley J. K. J. Neurosci. 1996, 16, 6106). It is thus believed that D3 receptor antagonists or partial agonists are effective medicaments for facilitating abstinence and for reducing relapse risk.

Parkinson's disease is a pathology characterized by resting tremors, limb rigidity and akinesia (difficulty initiating movements). The disease is caused by the degeneration of dopaminergic neurons. Treatment of Parkinson's disease is based on the substitution of dopamine by the administration of L-DOPA (3,4-dihydroxy-L phenylalanine) or direct agonists of dopamine. However, in many cases, the long term use of L-DOPA is associated with the appearance of abnormal movements, called dyskinesia. It has been shown in a non-human primate model of Parkinson's disease that modulating DRD3 with a highly selective partial agonist attenuates dyskinesia (Bezard E. Nat. Med. 2003, 6, 762). Consequently, the compounds disclosed in the present document are regarded as supplemental treatments for Parkinson's disease. Moreover, since it has been shown that DRD3 agonists increase neurogenesis in the rat, they may also be of use as medicaments which delay the development of the disease.

A mutation in the DRD3 gene is associated and cosegregates with essential tremor, a common hereditary neurological disorder, which is characterized by intention tremor in all or part of the body in the absence of another neurological pathology (Lucotte G. Clin. Genet. 2006, 69, 437-440). The mutation increases DRD3 functioning. Normalization of DRD3 functioning by using DRD3 antagonists or partial agonists could thus be an effective treatment for essential tremor.

As used above, the terms “dopamine D3 receptor,” “D3 receptor” or “DRD3” denotes a dopamine receptor subtype primarily expressed in the limbic system (Sokoloff P, Nature, 1990, 347, 146-151). DRD3 has been described in international patent WO 91/15513.

As used above, the term “D3 receptor partial agonist” denotes a compound that forms a complex with DRD3 and that acts as a combined agonist-antagonist, i.e., it induces a physiological response whose intensity is less than that of the natural mediator, dopamine. In vitro, in a cell expressing DRD3, a DRD3 partial agonist produced an active response whose maximum intensity was lower than that produced by dopamine or a by a full agonist, for example quinpirole[(4aR-trans)-4,4a,5,6,7,8,8a,9 octahydro-5-propyl-1H (or 2H)-pyrazolo(3,4-g)quinoline]. A DRD3 partial agonist can also partially prevent the response produced by dopamine or by its full agonists. In vivo, a DRD3 partial agonist produces dopaminergic responses, particularly when dopamine levels are lowered, as is the case with rats with lesions caused by 6-hydroxydopamine or monkeys injected with 1 methyl-4-phenyl-1,2,3,6-tetrahydropyridine (MPTP). Moreover, a DRD3 partial agonist can act as an antagonist in vivo, particularly when DRD3 is subject to sustained dopamine stimulation.

A “DRD3 antagonist” denotes a molecule that forms a complex with DRD3 and that is able to prevent a response triggered by dopamine or its agonists in a cell expressing DRD3.

As used here, the term “salts” designates inorganic acid and base addition salts of compounds of the present invention. Preferably, the salts are pharmaceutically acceptable, i.e., they are nontoxic for the patients to whom they are administered. Examples of acid addition salts include hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthalate salts and similar. (See, for example S. M. Berge et al., “Pharmaceutical salts,” J. Pharm. Sci., 66: p. 1-19 (1977)). Base addition salts include metal and amino pharmaceutically acceptable salts. Suitable metal salts contain sodium, potassium, calcium, barium, zinc, magnesium, and aluminum. Suitable amino base addition salts are prepared from amines, which are sufficiently basic to form a stable salt, and preferably include amines that are frequently used in medicinal chemistry due to their low toxicity in medical use. Such amines include ammonia, ethylenediamine, N-methyl-glucamine, ornithine, choline, N,N′-dibenzylethylenediamine, chloroprocaine, diethalolamine, procaine, N,N′-benzylphenethylamine, diethylamine, piperazine, dimethylamine, trimethylamine, ethylamine, bases made from amino acids, for example lysine and arginine, or dicyclohexylamine, and similar bases.

“Pharmaceutically acceptable” refers to molecular entities and compositions that do not produce adverse effects, allergies or other undesirable reactions when administered in animals or humans.

When used herein, the term “pharmaceutically acceptable excipient” includes any diluent, adjuvant or excipient, such as preservatives, fillers, disintegrants, wetting agents, emulsifiers, dispersants, antibacterials, antifungals, or agents which delay intestinal and digestive absorption and resorption. The use of such media or carriers is well-known to those persons skilled in the art. Except in the case where the agent is chemically incompatible with a chromene or thiochromene carboxamide derivative, its use in pharmaceutical compositions with the compounds according to the invention is envisaged.

In the context of the invention, the term “treatment,” as used herein, means the prevention or inhibition of the appearance or progression of the condition to which the term is applied, or to one or more symptoms of said condition.

“Therapeutically active quantity” means a quantity of a chromene or thiochromene carboxamide derivative that is effective for achieving the desired therapeutic effect according to the invention.

According to the invention, the term “patient” refers to a human or non-human mammal affected or susceptible to being affected by pathology. Preferentially, the patient is human.

The present invention relates to novel chromene and thiochromene carboxamide derivatives, methods for preparing same and the use of same as medicaments, as DRD3 receptor ligands, for the treatment of neurological or psychiatric diseases, conditions or disorders. Said novel compounds conform to general formula 1

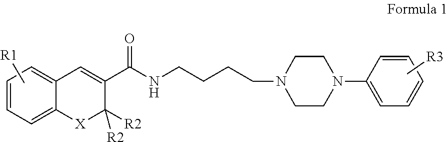

wherein:
X represents a heteroatom, O or S;
R1 represents an atom of hydrogen or one or more identical or different substituents on the homocycle such as halogen, Cl, F, Br or a C₁₋₄ alkoxy, OH, C₁₋₄ alkyl or CF₃ group;
R2 represents a hydrogen atom or C₁₋₄ alkyl group;
R3 represents a hydrogen atom or one or more identical or different substituents such as halogen, Cl, F, Br or a C₁₋₄ alkyl, C₁₋₄ alkoxy or thioalkoxy, O(CH₂)ₙO with n being 1 or 2, NO₂, NH₂, NHCOCH₃, NHSO₂CH₃, OH, CF₃, CN, COOEt or CH₂OH group, a phenyl or benzyl substituent optionally substituted, or R3 forms a ring fused with the aromatic ring which carries it, such as an aryl, heteroaryl or C₅, C₆ or C₇ cycloalkyl or a heterocycle.

The invention also relates to pharmaceutically acceptable water-soluble salts of compounds, possible enantiomers of same as well as pharmaceutical compositions containing same, and the use of same as a medicament for treating central nervous system disorders.

The chromene and thiochromene carboxamide compounds according to formula 1 are novel. The literature, such as patents WO 9929687 and WO 2000 075136, mentions benzopyranes or chromenes acting on stomach disorders. Compounds of 2-oxo-2H-chromene-3-carboxylic structure have been reported in J. Med. Chem. 2003, 46, 3883. Patents WO 2004 004729 and WO 2003 028728 describe butyl phenyl piperazine heteroaryl carboxamides as D3 ligands and We 2006 008133 describes nicotine receptor modulators, but these documents in no way mention the inventive chromenes or thiochromenes of the present invention.

The fact of introducing an oxygen or sulfur heteroatom within the ring structure, thus forming a chromene or thiochromene, shows the advantage of these compounds in making D3 dopamine antagonists or partial agonists.

The preferred compounds are as follows:
2H Chromene 3 carboxylic acid {4 [4-(2-methoxyphenyl)-piperazin-1-yl]-butyl}-amide
2H-Chromene-3-carboxylic acid {4-[4-(4-methoxyphenyl)piperazin-1-yl]-butyl}-amide
2H-Chromene-3-carboxylic acid {4-[4-(2-fluorophenyl)-piperazin-1-yl]-butyl}-amide
2H-Chromene-3-carboxylic acid {4-[4-(4-fluorophenyl)-piperazin-1-yl]-butyl}-amide
2H-Chromene-3-carboxylic acid {4-[4-phenylpiperazin-1-yl]-butyl}-amide
2H-Chromene-3-carboxylic acid {4-[4-(2-chlorophenyl)-piperazin-1-yl]-butyl}-amide
2H-Chromene-3-carboxylic acid {4-[4-(4-chlorophenyl)-piperazin 1 yl]-butyl}-amide
2H-Chromene 3 carboxylic acid {4-[4-(2,3-dichlorophenyl)piperazin-1-yl]-butyl}-amide
2H-Chromene-3-carboxylic acid {4-[4-(3-chlorophenyl)-piperazin-1-yl]-butyl}-amide
2H-Chromene-3-carboxylic acid {4-[4-(3-trifluoromethylphenyl)-piperazin-1-yl]-butyl}-amide
2H-Chromene-3-carboxylic acid {4-[4-(2-trifluoromethylphenyl)-piperazin-1-yl]-butyl}-amide
2H-Chromene-3-carboxylic acid {4-[4-(4-trifluoromethylphenyl)-piperazin-1-yl]-butyl}-amide
2H-Chromene-3-carboxylic acid {4-[4-(4-nitrophenyl)-piperazin-1-yl]-butyl}-amide
2H-Chromene 3 carboxylic acid {4-[4-(3-nitrophenyl)piperazin-1-yl]-butyl}-amide
2H-Chromene-3-carboxylic acid {4-[4-(3-aminophenyl)-piperazin-1-yl]-butyl}-amide
2H-Chromene-3-carboxylic acid {4-[4-(3-acetamidophenyl)piperazin-1-yl]-butyl}amide
2H-Chromene-3-carboxylic acid {4-[4-(3-methylsulfonamidophenyl)piperazin-1-yl]-butyl}-amide
2H-Chromene-3-carboxylic acid {4-[4-(2-nitrophenyl)-piperazin-1-yl]-butyl}-amide
2H-Chromene-3-carboxylic acid {4-[4-(2,3-dimethylphenyl)-piperazin-1-yl]-butyl}-amide
2H-Chromene-3-carboxylic acid {4-[4-(3,4-dimethylphenyl)-piperazin-1-yl]-butyl}-amide
2H-Chromene-3-carboxylic acid {4-[4-(2,4-dimethylphenyl)-piperazin-1-yl]-butyl}-amide
2H-Chromene-3-carboxylic acid {4-[4-(2-methylphenyl)-piperazin-1-yl]-butyl}-amide
2H-Chromene-3-carboxylic acid {4-[4-(3-methoxyphenyl)-piperazin-1-yl]-butyl}-amide
2H-Chromene-3-carboxylic acid {4-[4-(2-hydroxyphenyl)-piperazin-1-yl]-butyl}-amide
2H-Chromene-3-carboxylic acid {4-[4-(3-hydroxyphenyl)-piperazin-1-yl]-butyl}-amide
2H-Chromene-3-carboxylic acid {4-[4-(4-hydroxyphenyl)-piperazin-1-yl]-butyl}-amide
2H-Chromene-3-carboxylic acid {4-[4-(3,4-methylenedioxyphenyl)-piperazin-1-yl]-butyl}amide
2H-Chromene-3-carboxylic acid {4-[4-(3,4-dimethoxyphenyl)-piperazin-1-yl]-butyl}-amide
2H-Chromene-3-carboxylic acid {4-[4-(3,5-dimethoxyphenyl)-piperazin-1-yl]-butyl}-amide
2H-Chromene-3-carboxylic acid {4-[4-(2-cyanophenyl)-piperazin-1-yl]-butyl}-amide
2H-Chromene-3-carboxylic acid {4-[4-(3-cyanophenyl)-piperazin-1-yl]-butyl}-amide
2H-Chromene-3-carboxylic acid {4-[4-(4-cyanophenyl)-piperazin-1-yl]-butyl}-amide
2H-Chromene-3-carboxylic acid {4-[4-(3-ethoxycarbonylphenyl)-piperazin-1-yl]-butyl}amide
2H-Chromene-3-carboxylic acid {4-[4-(4-ethoxycarbonylphenyl)-piperazin-1-yl]-butyl}-amide
2H-Chromene-3-carboxylic acid {4-[4-(3-hydroxymethylphenyl)-piperazin-1-yl]-butyl}-amide
6-Methoxy-2H-chromene-3-carboxylic acid {4-[4-(2-methoxyphenyl)-piperazin-1-yl]-butyl}amide
6-Methoxy-2H-chromene-3-carboxylic acid {4-[4-(2-fluorophenyl)-piperazin-1-yl]-butyl}-amide
6-Methoxy-2H-chromene-3-carboxylic acid {4-[4-(2,3-dichlorophenyl)piperazin-1-yl]-butyl}-amide
6-Methoxy 2H-chromene-3-carboxylic acid {4-[4-(3-methoxyphenyl)-piperazin-1-yl]-butyl}-amide
6-Methoxy-2H-chromene-3-carboxylic acid {4-[4-(4-methoxyphenyl)-piperazin-1-yl]-butyl}-amide
6-Methoxy-2H-chromene-3-carboxylic acid {4-[4-(3-hydroxyphenyl)-piperazin-1-yl]-butyl}-amide
6-Methoxy-2H-chromene-3-carboxylic acid {4-[4-(3-cyanophenyl)-piperazin-1-yl]-butyl}-amide
6-Methoxy-2H-chromene-3-carboxylic acid {4-[4-(4-cyanophenyl)-piperazin-1-yl]-butyl}-amide
6-Methoxy-2H-chromene-3-carboxylic acid {4-[4-(2-cyanophenyl)-piperazin-1-yl]-butyl}-amide
6-Methoxy-2H-chromene-3-carboxylic acid {4-[4-(3-ethoxycarbonylphenyl)-piperazin-1-yl]-butyl}-amide
6-Methoxy 2H-chromene-3-carboxylic acid {4-[4-(3-hydroxymethylphenyl)piperazin-1-yl]-butyl}-amide
6-Methoxy-2H-chromene-3-carboxylic acid {4-[4-(3,4-dimethoxyphenyl)-piperazin-1-yl]-butyl}-amide
6-Methoxy-2H-chromene-3-carboxylic acid {4-[4-(3,4-methylenedioxyphenyl)piperazin-1-yl]-butyl}-amide
2,2-Dimethyl-2H-chromene-3-carboxylic acid {4-[4-(3-hydroxyphenyl)-piperazin-1-yl]-butyl}-amide
2,2-Dimethyl-2H-chromene-3-carboxylic acid {4-[4-(2-cyanophenyl)-piperazin-1-yl]-butyl}-amide
2,2-Dimethyl-2H-chromene-3-carboxylic acid {4-[4-(3-cyanophenyl)piperazin-1-yl]-butyl}-amide
2,2-Dimethyl-2H-chromene-3-carboxylic acid {4-[4-(3-hydroxymethylphenyl)-piperazin-1-yl]-butyl}-amide
2H-Thiochromene-3-carboxylic acid {4-[4-(2-methoxyphenyl)-piperazin-1-yl]-butyl}-amide
2H-Thiochromene-3-carboxylic acid {4-[4-(2-fluorophenyl)-piperazin-1-yl]-butyl}-amide
2H-Thiochromene-3-carboxylic acid {4-[4-(2-chlorophenyl)-piperazin-1-yl]-butyl}-amide
2H-Thiochromene-3-carboxylic acid {4-[4-(2,3-dichlorophenyl)-piperazin-1-yl]-butyl}-amide
2H-Thiochromene-3-carboxylic acid {4-[4-(3-chlorophenyl)-piperazin-1-yl]-butyl}-amide
2H-Thiochromene-3-carboxylic acid {4-[4-(3-trifluoromethylphenyl)-piperazin-1-yl]-butyl}-amide
2H-Thiochromene-3-carboxylic acid {4-[4-(3-methoxyphenyl)-piperazin-1-yl]-butyl}-amide
2H-Thiochromene-3-carboxylic acid {4-[4-(4-methoxyphenyl)-piperazin-1-yl]-butyl}-amide
2H-Thiochromene-3-carboxylic acid {4-[4-(3,4-dimethoxyphenyl)-piperazin-1-yl]-butyl}-amide
2H-Thiochromene-3-carboxylic acid {4-[4 (3-hydroxyphenyl)-piperazin-1-yl]-butyl}-amide
2H-Thiochromene-3-carboxylic acid {4-[4-(2 hydroxyphenyl)-piperazin-1-yl]-butyl}-amide
2H-Thiochromene-3-carboxylic acid {4-[4-(4-hydroxyphenyl)piperazin-1-yl]-butyl}-amide
2H-Thiochromene-3-carboxylic acid {4-[4-(2 cyanophenyl)-piperazin-1-yl]-butyl}-amide
2H-Thiochromene-3-carboxylic acid {4-[4-(4-cyanophenyl)-piperazin-1-yl]-butyl}-amide
2H-Thiochromene-3-carboxylic acid {4-[4-(3 cyanophenyl)-piperazin-1-yl]-butyl}-amide
2H-Thiochromene-3-carboxylic acid {4-[4-(3 methoxycarbonylphenyl)-piperazin-1-yl]-butyl}-amide
2H-Thiochromene-3-carboxylic acid {4-[4-(3 hydroxymethylphenyl)-piperazin-1-yl]-butyl}-amide
6-Methoxy-2H-thiochromene-3-carboxylic acid {4-[4-(2-methoxyphenyl)-piperazin-1-yl]-butyl}-amide
6-Methoxy-2H-thiochromene-3-carboxylic acid {4-[4-(2-fluorophenyl)-piperazin-1-yl]-butyl}-amide
6-Methoxy-2H-thiochromene-3-carboxylic acid {4-[4-(3-chlorophenyl)-piperazin-1-yl]-butyl}-amide
6-Methoxy-2H-thiochromene-3-carboxylic acid {4-[4-(3-trifluoromethylphenyl)-piperazin-1-yl]-butyl}-amide
6-Methoxy-2H-thiochromene-3-carboxylic acid {4-[4-(3-methoxyphenyl)-piperazin-1-yl]-butyl}-amide
6-Methoxy-2H-thiochromene-3-carboxylic acid {4-[4-(3,4-dimethoxyphenyl)-piperazin-1-yl]-butyl}-amide
6-Methoxy-2H-thiochromene-3-carboxylic acid {4-[4-(3-hydroxyphenyl)-piperazin-1-yl]-butyl}-amide
6-Methoxy-2H-thiochromene-3-carboxylic acid {4-[4-(3-cyanophenyl)-piperazin-1-yl]-butyl}-amide
6-Methoxy-2H-thiochromene-3-carboxylic acid {4-[4-(3-ethoxycarbonylphenyl)-piperazin-1-yl]-butyl}-amide
6-Methoxy-2H-thiochromene-3-carboxylic acid {4-[4-(3-hydroxymethylphenyl)-piperazin-1-yl]-butyl}-amide
2,2-Dimethyl 2H-thiochromene-3-carboxylic acid {4-[4-(2-cyanophenyl)piperazin 1-yl]-butyl}-amide
2,2-Dimethyl-2H-thiochromene-3-carboxylic acid {4-[4-(3-hydroxymethylphenyl)-piperazin-1-yl]-butyl}-amide
2,2-Dimethyl-2H-thiochromene-3-carboxylic acid {4-[4-(2-methoxyphenyl)-piperazin-1-yl]-butyl}-amide
2,2-Dimethyl-2H-thiochromene-3-carboxylic acid {4-[4-(2-fluorophenyl)-piperazin-1-yl]-butyl}-amide
2,2-Dimethyl-2H-thiochromene-3-carboxylic acid {4 [4-(2,3-dichlorophenyl)-piperazin-1-yl]-butyl}-amide
2,2-Dimethyl-2H-thiochromene-3-carboxylic acid {4-[4-(3-chlorophenyl)piperazin-1-yl]-butyl}-amide
2,2-Dimethyl-2H-thiochromene-3-carboxylic acid {4-[4-(3-hydroxyphenyl)piperazin-1-yl]-butyl}-amide
2,2-Dimethyl 2H-thiochromene-3-carboxylic acid {4-[4-(3-cyanophenyl)-piperazin-1-yl]-butyl}-amide
5-Bromo-8-methoxy-2H-chromene-3-carboxylic acid {4-[4-(2-methoxyphenyl)piperazin-1-yl]-butyl}-amide
5-Bromo-8-methoxy-2H-chromene-3-carboxylic acid {4-[4-(2,3-dichlorophenyl)piperazin-1-yl]-butyl}-amide
6-Chloro-2H-chromene-3-carboxylic acid {4-[4-(2-methoxyphenyl)-piperazin-1-yl]-butyl}-amide
6-Chloro-2H-chromene-3-carboxylic acid {4-[4-(2,3-dichloro-methoxyphenyl)piperazin-1-yl]-butyl}-amide
6-Chloro-2H-chromene-3-carboxylic acid {4-[4-(2-fluorophenyl)-piperazin-1-yl]-butyl}-amide
6-Chloro-2H-chromene-3-carboxylic acid {4-[4-(2 cyanophenyl)-piperazin-1-yl]-butyl}-amide
6-Chloro-2H-chromene-3-carboxylic acid {4-[4-(3-cyanophenyl)-piperazin-1-yl]-butyl}-amide
6-Chloro-2H-chromene-3-carboxylic acid {4-[4-(4-cyanophenyl)-piperazin-1-yl]-butyl}-amide
6-Chloro-2H-chromene-3-carboxylic acid {4-[4-(3-hydroxyphenyl)-piperazin-1-yl]-butyl}-amide
6-Chloro-2H-chromene-3-carboxylic acid {4-[4-(3,4-dimethoxy phenyl)-piperazin-1-yl]-butyl}-amide
6-Chloro-2H-chromene-3-carboxylic acid {4-[4-(3,4-methylenedioxy phenyl)piperazin-1-yl]-butyl}-amide
7-methoxy-2H-chromene-3-carboxylic acid {4-[4-(2-methoxy-phenyl)-piperazin-1-yl]-butyl}-amide
7-methoxy-2H-chromene-3-carboxylic acid {4-[4-(3 cyano-phenyl)-piperazin-1-yl]-butyl}-amide
7-methoxy-2H-chromene-3-carboxylic acid {4-[4-(2,3-dichloro-phenyl)-piperazin-1-yl]-butyl}-amide
7-methoxy-2H-chromene-3-carboxylic acid {4-[4-(3-hydroxy-phenyl)-piperazin-1-yl]-butyl}-amide
7-methoxy-2H-chromene-3-carboxylic acid {4-[4-(2,3-dihydro benzo[1,4]dioxin-6-yl)-piperazin-1-yl]-butyl}-amide
7-methoxy-2H-chromene-3-carboxylic acid {4-[4-(3-methyloxy-carbonyl-)-piperazin-1-yl]-butyl}-amide
6-methoxy-2H-chromene-3-carboxylic acid {4-[4-(2,4-dichloro-phenyl)-piperazin-1-yl]-butyl}-amide
6-methoxy-2H-chromene-3-carboxylic acid {4-[4-(3-amino-phenyl)-piperazin-1-yl]-butyl}-amide
6-methoxy-2H-chromene-3-carboxylic acid {4-[4-(3-nitro-phenyl)-piperazin-1-yl]-butyl}-amide
6-methoxy-2H-chromene-3-carboxylic acid {4-[4-(3 acetylamino-phenyl)-piperazin-1-yl]-butyl}-amide
6-methoxy-2H-chromene-3-carboxylic acid {4-[4-(2,3-benzo-1,4-dioxanyl-)-piperazin-1-yl]-butyl}-amide
6-methoxy-2H-chromene-3-carboxylic acid {4-[4-(3,4-benzo-1,4-dioxanyl)-piperazin-1-yl]-butyl}-amide
6-methoxy-2H-chromene-3-carboxylic acid {4-[4-(2-oxo 2,3-dihydro-1H-benzimidazol-4-yl)-piperazin-1-yl]-butyl}-amide
6-methoxy-2H-chromene-3-carboxylic acid {4-[4-(3H-benzimidazol-4-yl)-piperazin-1-yl]-butyl}-amide
6-methoxy-2H-chromene-3-carboxylic acid {4-[4-(2-oxo-2,3-dihydro-1H-benzoxazol-7-yl)-piperazin-1-yl]-butyl}-amide
6-methoxy-2H-chromene-3-carboxylic acid {4-[4-(3-methylamino-carbonyl)-piperazin-1-yl]-butyl}-amide
6-methoxy-2H-chromene-3 carboxylic acid {4-[4-(3-mesylamino-phenyl)piperazin-1-yl]-butyl}-amide
6-chloro-2H-chromene-3-carboxylic acid {4-[4-(2,4-dichloro-phenyl)-piperazin-1-yl]-butyl}-amide
6-chloro-2H-chromene-3-carboxylic acid {4-[4 (3-nitro-phenyl)-piperazin-1-yl]-butyl}-amide
6-chloro-2H-chromene-3-carboxylic acid {4-[4-(3-amino-phenyl)piperazin-1-yl]-butyl}-amide
6-chloro-2H-chromene-3-carboxylic acid {4-[4-(3-acetylamino-phenyl)-piperazin-1-yl]-butyl}-amide
6-chloro-2H-chromene-3-carboxylic acid {4-[4-(3-hydroxymethyl-phenyl)-piperazin-1-yl]-butyl}-amide
6-chloro-2H-chromene-3-carboxylic acid {4-[4-(3-mesylamino-phenyl)piperazin-1-yl]-butyl}-amide
6-fluoro-2H-chromene-3-carboxylic acid {4-[4-(2,3-dichloro-phenyl)-piperazin-1-yl]-butyl}-amide
6-fluoro-2H-chromene-3-carboxylic acid {4-[4-(2-methoxy-phenyl)-piperazin-1-yl]-butyl}-amide
6-fluoro-2H-chromene-3-carboxylic acid {4-[4-(3-cyano-phenyl)-piperazin-1-yl]-butyl}-amide
6-fluoro-2H-chromene-3-carboxylic acid {4-[4-(3-acetylamino-phenyl)-piperazin-1-yl]-butyl}-amide
6-fluoro-2H-chromene-3-carboxylic acid {4-[4-(3-hydroxy-phenyl)-piperazin-1-yl]-butyl}-amide
6-fluoro-2H-chromene-3-carboxylic acid {4-[4 (3-nitro-phenyl)-piperazin-1-yl]-butyl}-amide
6-fluoro-2H-chromene-3 carboxylic acid {4-[4-(3-mesylamino phenyl)piperazin-1-yl]-butyl}-amide
6-fluoro-2H-chromene-3-carboxylic acid {4-[4-(3-amino-phenyl)-piperazin-1-yl]-butyl}-amide
6-fluoro-2H-chromene-3-carboxylic acid {4-[4-(3-methylcarbamoyl-phenyl)-piperazin-1-yl]-butyl}-amide
6-fluoro-2H-chromene-3-carboxylic acid {4-[4-(2,3-benzo-1,4-dioxanyl)-piperazin-1-yl]-butyl}-amide
6-fluoro-2H-chromene-3-carboxylic acid {4-[4-(2-oxo-2,3-dihydro-1H-benzimidazol-4-yl)-piperazin-1-yl]-butyl}-amide
6-fluoro-2H-chromene-3-carboxylic acid {4-[4-(3H-benzimidazol-4-yl)-piperazin-1-yl]-butyl}-amide
6-fluoro-2H-chromene-3-carboxylic acid {4-[4-(2-oxo-2,3-dihydro-1H-benzoxazol-7-yl)-piperazin-1-yl]-butyl}-amide
6-fluoro-2H-chromene-3-carboxylic acid {4-[4-(3-methyloxy-carbonyl)-piperazin-1-yl]-butyl}-amide
6-fluoro-5 (4-{4-[2H-chromene-3-carbonyl)amino]-butyl}-piperazin-1-yl)-benzofuran-2-carboxylic acid methyl ester
2H-chromene-3-carboxylic acid {4-[4-(3,4,5-trimethoxy phenyl)-piperazin-1-yl]-butyl}-amide
2H-chromene-3-carboxylic acid {4-[4-(1H-indol-4-yl)-piperazin-1-yl]-butyl}-amide
2H-chromene-3-carboxylic acid {4-[4-(2,3-dihydro benzo[1,4]dioxin-6-yl)-piperazin-1-yl]-butyl}-amide
2H-chromene-3-carboxylic acid {4-[4-(2,3-dihydro-benzo[1,4]dioxin-5-yl)-piperazin-1-yl]-butyl}-amide
5-(4-{4-[2H-chromene-3-carbonyl)-amino]butyl}-piperazin-1-yl)-benzofuran-2-carboxylic acid methyl ester
2H-chromene-3-carboxylic acid {4-[4-(2,3-dihydro-1H-indol-4-yl)-piperazin-1-yl]-butyl}-amide
2H-chromene-3-carboxylic acid {4-[4-(3-mesylamino-phenyl)piperazin-1-yl]-butyl}-amide
2H-chromene-3-carboxylic acid {4-[4-(1-acetyl-2,3-dihydro-1-H-indol-4-yl)-piperazin-1-yl]-butyl}-amide
2H-chromene-3-carboxylic acid {4-[4-(2-oxo-2,3-dihydro-benzoxazol-7-yl)piperazin-1-yl]-butyl}-amide
2H-chromene-3-carboxylic acid {4-[4-(2-oxo-2,3-dihydro-1H-benzimidazol-4-yl)-piperazin-1-yl]-butyl}-amide
2H-chromene-3-carboxylic acid {4-[4-(3H-benzimidazol-4-yl)-piperazin-1-yl]-butyl}-amide
2H-chromene-3-carboxylic acid {4-[4-(3-carbamoyl-phenyl)-piperazin-1-yl]-butyl}-amide
2H-chromene-3-carboxylic acid {4-[4-(3-methylcarbamoyl-phenyl)-piperazin-1-yl]-butyl}-amide
2H-chromene-3-carboxylic acid {4-[4-(2,3-dihydro-benzofuran-7-yl)-piperazin-1-yl]-butyl}-amide
2H-thiochromene-3-carboxylic acid {4-[4-(2,3-dimethyl)-piperazin-1-yl]-butyl}-amide
2H-thiochromene-3-carboxylic acid {4-[4-(3-methyl-phenyl)-piperazin-1-yl]-butyl}-amide
2H-thiochromene-3-carboxylic acid {4-[4-(4-chloro-phenyl)-piperazin-1-yl]-butyl}-amide
2H-thiochromene-3-carboxylic acid {4-[4-(2,4-dimethoxy-phenyl)-piperazin-1-yl]-butyl}-amide
2H-thiochromene-3-carboxylic acid {4-[4-(3-formyl-phenyl)-piperazin-1-yl]-butyl}-amide
2H-thiochromene-3-carboxylic acid {4-[4-(3-mesylamino-phenyl)-piperazin-1-yl]-butyl}-amide
2H-thiochromene-3-carboxylic acid {4-[4-(3 nitro-phenyl)-piperazin-1-yl]-butyl}-amide
5-(4-{4-[2H-thiochromene-3-carbonyl)-amino]-butyl}-piperazin-1-yl)-benzofuran-2-carboxylic acid methyl ester
2H-thiochromene-3-carboxylic acid {4-[4-(2-oxo-2,3-dihydro-1H-benzimidazol-4-yl)piperazin-1-yl]-butyl}-amide
2H-thiochromene-3-carboxylic acid {4-[4-(3H-benzimidazol-4-yl)-piperazin-1-yl]-butyl}-amide
2H-thiochromene-3-carboxylic acid {4-[4-(2-oxo-2,3-dihydro-benzoxazol-7-yl)piperazin-1-yl]-butyl}-amide
2H-thiochromene-3-carboxylic acid {4-[4-(3-methylcarbamoyl-phenyl)piperazin-1-yl]-butyl}-amide
2H-thiochromene-3-carboxylic acid {4-[4-(3-carbamoyl-phenyl)-piperazin-1-yl]-butyl}-amide
2H-thiochromene-3-carboxylic acid {4-[4-(2,3-dihydro-benzo[1,4]dioxin-6-yl)-piperazin-1-yl]-butyl}-amide
2H-thiochromene-3-carboxylic acid {4-[4-(3-acetylamino-phenyl)-piperazin-1-yl]-butyl}-amide
2H-thiochromene-3-carboxylic acid {4-[4-(2,3-dihydro-benzo[1,4]dioxin-5-yl)-piperazin-1-yl]-butyl}-amide
6-chloro-2H-thiochromene-3-carboxylic acid {4-[4-(2,3-dihydro-benzo[1,4]dioxin-6-yl)piperazin-1-yl]-butyl}-amide
6-chloro-2H-thiochromene-3-carboxylic acid {4-[4-(3-cyano-phenyl)-piperazin-1-yl]-butyl}-amide
6-chloro-2H-thiochromene-3-carboxylic acid {4-[4-(3-chloro-phenyl)-piperazin-1-yl]-butyl}-amide
6-chloro-2H-thiochromene-3-carboxylic acid {4-[4-(3-hydroxy-phenyl)-piperazin-1-yl]-butyl}-amide
6-chloro-2H-thiochromene-3-carboxylic acid {4-[4-(2-methoxy-phenyl)-piperazin-1-yl]-butyl}-amide
6-chloro-2H-thiochromene-3-carboxylic acid {4-[4-(2-fluoro-phenyl)piperazin-1-yl]-butyl}-amide
6-chloro-2H-thiochromene-3-carboxylic acid {4-[4-(2,4-dimethoxy-phenyl)-piperazin-1-yl]-butyl}-amide
6-Methoxy-2H-thiochromene-3-carboxylic acid {4-[4-(3-mesylamino-phenyl)piperazin-1-yl]-butyl}-amide
6-Fluoro 2H-thiochromene-3-carboxylic acid {4-[4-(3-mesylamino-phenyl)-piperazin-1-yl]-butyl}-amide
6-Chloro-2H-thiochromene-3-carboxylic acid {4-[4-(3-mesylamino-phenyl)-piperazin-1-yl]-butyl}-amide

The present invention also relates to methods for preparing said compounds.

The compounds of general formula 1 are prepared from chromene or thiochromene acids of formula 2 by conventional peptide coupling with substituted 4-(4-phenylpiperazin-1-yl)-butylamine. The great diversity of peptide coupling methods described in the literature (Tet. 2005, 61, 10827) leaves to those skilled in the art the choice of applying the most efficient method and providing the purest compound (SOCl₂, oxalyl chloride/DMF, DCC, mixed anhydrides, CDI, BOP and derivatives thereof, TBTU, etc.).

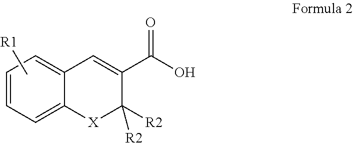

The chromene carboxylic acids of formula 2 (X═O, R2=H) are obtained (diagram 1) by the reaction of R1 substituted salicylic aldehydes with acrylonitrile in the presence of DABCO or DBU, by a Bayliss-Hillman reaction, according to the method of Wise, J. Med. Chem. 1988, 31, 688, or Bioorg. Med. Chem. Lett. 1996, 6, 1077, or of Shiraishi, J. Med. Chem. 2000, 43, 2049, in the presence of t-BuOK. Base hydrolysis provides the corresponding acids 2 (X═O, R2=H).

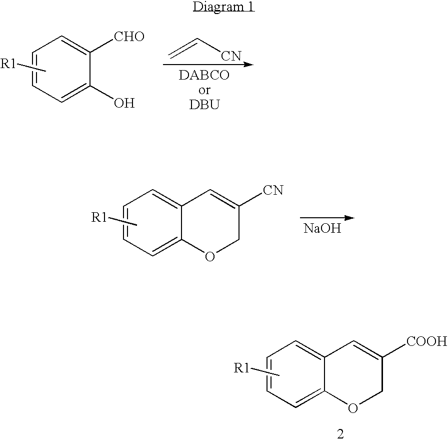

The corresponding thiochromene acids of formula 2 (X═S, R2=H) are obtained in 3 steps (diagram 2) from 2-mercaptobenzoic acids by reduction with LAH in 2-mercaptobenzylic alcohols 3, then by oxidation with MnO₂ in toluene in thiosalicylic aldehyde in the form of dimer 4, according to Synthesis 1989, 763. The condensation of this intermediate aldehyde 4 with acrylonitrile according to the method analogous to that of chromene acids (Synthesis, 2001, 2389) provides the thiochromene acids 2 (X═S, R2=H).

Similarly, the chromene or thiochromene carboxylic acids substituted in position 2 of formula 2 (X═O or S, R2=Me) are obtained with 3,3-dimethyl-acrylonitrile instead of acrylonitrile.

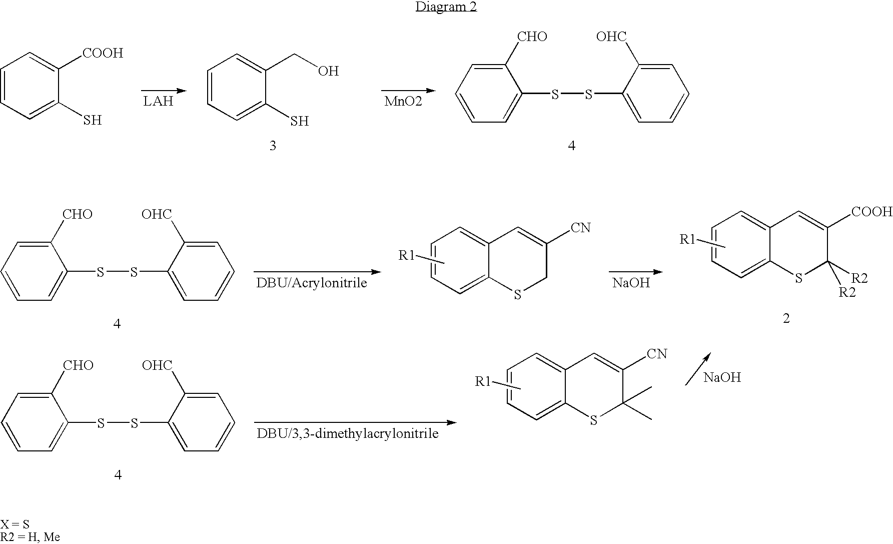

The substituted 4-(4-phenylpiperazin-1-yl)-butylamines of formula 5 are obtained (diagram 3) according to the various common methods described in the literature, such as for example J. Med. Chem. 2001, 44, 3175, (method via the hydrazinolysis of phthalimidobutyl piperazine derivative 6), or Bioorg. Med. Chem. Lett. 2004, 14, 195, (method via the reduction by LAH of nitrile 7), or J. Med. Chem. 2003, 46, 3883 (method by reduction of nitrile 7 with Ni-Raney), or finally J. Med. Chem. 2002, 45, 5727 (method by reduction of nitrile 7 with B₂H₆/dimethyl sulfide). These various methods are selected according to the substituents carried by the phenyl ring attached to the piperazine.

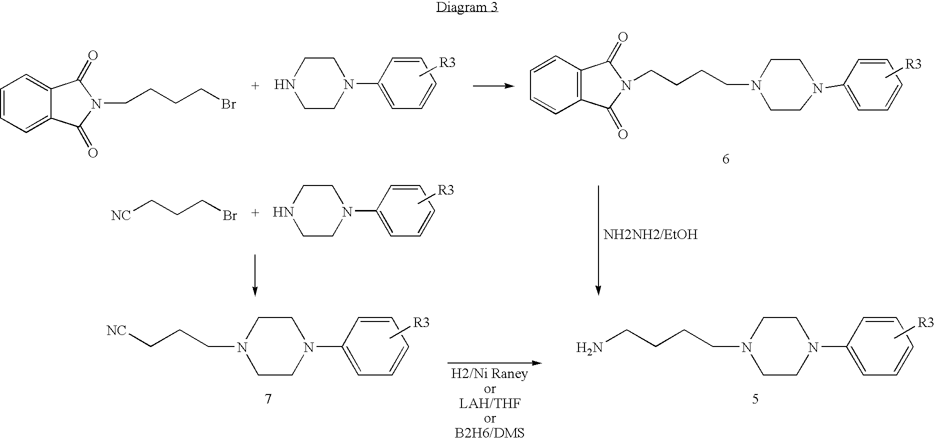

### Preparation:

The variously-substituted 4-phenyl-piperazines or 4-(4-phenylpiperazin-1-yl)-butylamines are prepared according to the various methods described in the literature. The 4-acetyl, mesyl or amino-phenyl piperazine derivatives are prepared in several steps. Catalytic hydrogenation in the presence of palladium of nitro-phenyl piperazine as described by Lopez-Rodriguez (J. Med. Chem. 2001, 44, 186-197) yields the aniline intermediate that is acylated or mesylated by acetyl chloride or mesyl chloride in basic medium as described by Orus (Pharmazie, 2002, 57, 515-518). The preparation of heterocyclic phenyl-piperazines such as benzimidazolone, benzimidazolyl-piperazine, benzoxazolone piperazine, derivatives such as benzo-1,4-dioxanyl or dihydro-indolyl-piperazine proceeds according to methods described in patents WO 9736893 or EPO 189612. Similarly, Devlin (Synth. Commun. 1995, 25, 711-718) described the method for preparing benzimidazole from 1,2-diaminobenzene which we selected.

The coupling of chromene or thiochromene acids of formula 2 with the primary amines of formula 5 is carried out under standard peptide coupling conditions, preferentially by the methods described in J. Org. Chem. 1996, 61, 2322, Bioorg. Med. Chem. 2005, 13, 519, Org. Lett. 2005, 7 (16) 3481, and J. Org. Chem. 2006, 71, 3364.

The compounds of formula 1, wherein R3 represents a hydroxymethyl or phenol group, can also be synthesized according to diagram 4:

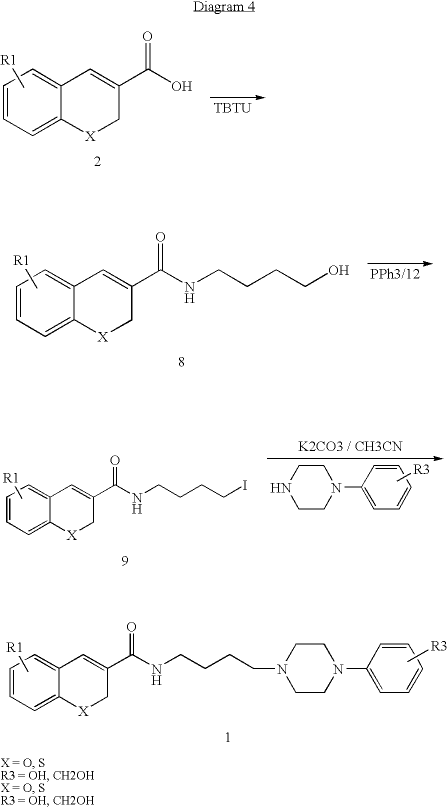

The chromene or thiochromene carboxylic acids of formula 2 are initially amidated with the amino butanol in the intermediate of formula 8, then iodized with PPh₃/I₂ in the compound of formula 9 according to J. Chem. Soc. Perkin Trans I, 2000, 219. Conventional condensation (K₂CO₃/CH₃CN) with substituted phenylpiperazines provide the compounds of formula 1 according to the same method as that described in J. Med. Chem. 2003, 46, 3822.

The activity of derivatives of formula 1 as DRD3 ligands, modulators of the activity of DRD3 according to the invention, was evaluated on cells expressing human recombinant DRD3. The inventors demonstrated that the compounds of formula 1 behave as powerful ligands, with inhibition constants (Kᵢ) from 0.1 to 10 nM/l⁻¹. These same compounds show an apparent affinity for the dopamine D2 receptor that is 100 to 500 times weaker. These same compounds have an affinity for the α₁ adrenergic receptor that is 20 to 500 times weaker than that for the D3 receptor. The compounds of formula 1 are either antagonists (intrinsic activity<0.10), partial agonists (0.2<intrinsic activity<0.6) or full agonists (intrinsic activity>0.8). The biological results relative to certain compounds of formula 1 are presented in table 2 at the end of the description.

By taking into account the selective modulation of dopamine signals which DRD3 exerts in limbic regions, which are implicated in emotional and cognitive processes, the inventive compounds are suitable for various therapeutic applications and do not interfere with dopaminergic signals of the extrapyramidal, ante-hypophyseal or vegetative systems (the area postrema, for example). Consequently, the inventive compounds are free of the side effects of existing compounds, which result from blockage of D2 receptors expressed in the extrapyramidal, ante hypophyseal and vegetative systems. The inventive derivatives can thus be used for preparing pharmaceutical compositions and medicaments for treating neurological or psychiatric diseases, conditions or disorders involving DRD3, such as psychotic states.

In addition, since one effect of antidepressants is to increase expression of DRD3 in areas of the brain involved in motivation, the compounds can mimic the action of antidepressants. The inventive derivatives can thus be used for preparing pharmaceutical compositions and medicaments for treating depression.

Taking into account the role of DRD3 in drug dependency, the pharmaceutical compositions or medicaments based on the derivatives described in the present invention can be usefully administered for states related to abstinence and/or to facilitate detoxification in patients dependent on or addicted to cocaine, heroin, alcohol, tobacco, and other addictive substances.

In the same way as DRD3 partial agonists in general, the derivatives according to the invention can also be used as a supplemental treatment to the treatment of Parkinson's disease by L-DOPA.

In the same way as DRD3 antagonists and partial agonists, the derivatives according to the invention can also be used to treat essential tremor.

Thus, the compounds of formula 1, or the acid or base salts thereof, can be used to treat neurological or psychiatric conditions, in particular conditions that can be treated by DRD3 antagonists, agonists or partial agonists.

Consequently, the invention also relates to a pharmaceutical composition that comprises at least one compound according to the invention, in combination with a conventional pharmaceutically acceptable excipient. The invention also relates to a method for treating neurological or psychiatric conditions, diseases or disorders, consisting of administering to a patient who requires treatment a compound of formula 1 in a therapeutically effective quantity. The invention also relates to compounds of formula 1 for the use thereof as a medicament and to the use of a compound of formula 1 for manufacturing a medicament for the treatment of a neurological or psychiatric disease or disorder.

Examples of conditions, diseases, or neurological or psychiatric disorders according to the invention include psychoses (schizophrenia in particular), depression, essential tremor, dependence on or addiction to various drugs or addictive substances such as tobacco or alcohol, cognitive deficits caused by aging or neurodegenerative diseases such as Alzheimer's disease, Parkinson's disease, dyskinesia, tardive dyskinesia or other movement disorders related to the use of medicaments used in the treatment of Parkinson's disease or schizophrenia.

The derivatives of formula 1 according to the invention can be administered by oral, systemic, parenteral, nasal or rectal route. In particular, the derivative can be administered by oral route in a suitable formulation. Formulations suitable for oral administration to a patient include therapeutic units such as capsules, packets or tablets, each containing a predetermined quantity of a compound of formula 1; such formulations also include powders or granules, solutions or suspensions in aqueous or non aqueous liquids, or oil-in-water liquid emulsions or water-in-oil liquid emulsions.

The amount of the compounds of formula 1 in the inventive compositions can be adjusted in order to have a quantity of active substance that is effective in achieving the desired therapeutic response using a composition specific to the administration method. The amount selected thus depends on desired therapeutic effect, administration route, treatment duration and other factors.

The total daily dosage of useful compounds according to the present invention administered in single or divided doses can be, for example, in the range of 0.001-100 mg per kilogram of body weight per day, preferably in the range of 0.01-10 mg/kg/day.

The specific dosage for a given patient will depend on a variety of factors including body weight, general health, sex, diet, administration duration and route, absorption, intestinal resorption and excretion rates, combination with other medicaments and the severity of the specific condition being treated.

Preparations of the inventive compounds are illustrated in the following non-limiting examples:

### EXAMPLE 1

### 2H-Chromene 3-carboxylic acid {4-[4-(2-methoxyphenyl)-piperazin-1-yl]-butyl}-amide

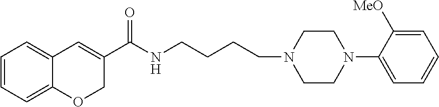

### Step 1: Preparation of 2-H-chromene-3-carbonitrile

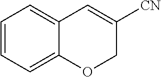

According to the method described in J. Med. Chem. 1988, 31, 688, 2-H-chromene is obtained in the following way: heat to 80° C. 15 g of salicylic aldehyde (0.123 mol) diluted in 50.8 g of acrylonitrile (0.958 mol) then add to the mixture 6.9 g of DABCO (0.061 mol). After 8 h of heating, allow the reaction mixture to return to room temperature. Add 100 ml of 1 N NaOH and then prepare three successive extractions with 50 ml of dichloromethane. After washing with water, drying on MgSO₄, filtration and concentration, an oil is obtained which is chromatographed on silica with dichloromethane eluent to give 10.5 g of 2H-chromene-3 carbonitrile in the form of white powder with a yield of 55%. ¹H NMR (DMSO): 4.88 (s, 2H, O—CH₂—), 6.90 (d, 1H, H arom), 7.03 (t, 1H, Haro), 7.31 (m, 2H, H arom), 7.58 (s, 1H, H₄).

### Step 2: Nitrile hydrolysis

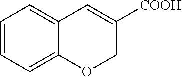

Heat at 100° C. 5 g of 2H-chromene-3-carbonitrile, obtained in the previous step, in solution in 50 ml of 10% NaOH. After 2 hours at reflux, the reaction mixture is allowed to return to room temperature and then a large volume of water (100 ml) is added. Acidification proceeds with care, at around 0-5° C., using concentrated HCl (up to pH 1). The acid precipitates in the aqueous phase and is recovered by filtration, washed with water and then dried under a vacuum. 5.4 g of 2H-chromene-3-carboxylic acid is obtained in the form of a cream-colored powder with a yield of 96%. ¹H NMR (DMSO): 4.90 (s, 2H, O—CH₂—), 6.85 (d, 1H, H arom), 6.95 (t, 1H, R arom), 7.25 (m, 2H, H arom), 7.44 (s, 1H, H₄), 12.55 (s, 1H, CO₂H).

### Step 3: Preparation of 2-{4-[4-(2-methoxy-phenyl)-piperazin-1-yl]-butyl}-isoindole-1,3-dione

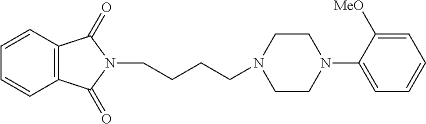

In 200 ml of acetonitrile dissolve successively 10 g of 1-(2-methoxyphenyl)piperazine (0.052 mol), 14.7 q of N-(4-bromobutyl)-phthalimide (0.052 mol). Add 7.2 g of K₂CO₃ (0.052 mol) and a KI crystal. The mixture is brought to acetonitrile reflux for 12 hours. After returning to room temperature and evaporation of the reaction medium, the result is taken up in 250 ml of water. Three dichloromethane extractions followed by drying on MgSO₄ and concentration allow isolation of a yellow oil which is taken up in 150 ml of isopropyl ether; after trituration the oil yields a precipitate that is isolated by filtration. After 2 washings with isopropyl ether, 17.7 q of 2-{4-[4-(2-methoxy-phenyl)piperazin-1-yl]-butyl}-isoindole-1,3-dione is isolated in the form of a white powder with a yield of 87%. This intermediate is used directly in step 4.

¹H NMR (CD₃OD): 1.52 (m, 2H, CH₂), 1.75 (m, 2H, CH₂), 2.47 (t, 2H, CH₂—Npip), 2.64 (m, 4H, piperazine), 3.03 (m, 4H, piperazine), 3.71 (t, 2H, CH₂-phthalimide), 3.76 (s, 3H, —OCH₃), 6.94 (m, 4H, Haro, arylpiperazine), 7.82 (m, 4H, arylphth.).

### Step 4: Preparation of 4-[4-(2-methoxy-phenyl)-piperazin-1-yl]-butylamine

17.7 g of 2-{4-[4-(2-methoxy-phenyl)-piperazin-1-yl]-butyl}-isoindole-1,3-dione (0.045 mol), prepared in previous step 3, is dissolved in 200 ml of absolute ethanol. 8.8 ml of a hydrated hydrazine solution (0.180 mol) is added and the mixture is carried at ethanol reflux for 6 h. A white precipitate is formed. After returning to room temperature, the precipitate is filtered, rinsed with ethanol and the organic filtrate evaporated. The residue obtained is taken up in 150 ml of dichloromethane and then washed twice with an equivalent volume of water. After drying and then concentrating the organic phase, 4-[4-(2-methoxy-phenyl)-piperazin-1-yl]-butylamine is obtained in the form of a yellow oil with a yield of 65%. This amine is used directly in amide formation step 5. ¹H NMR (CD₃OD): 1.52 (m, 4H, —CH₂—CH₂), 2.42 (m, 2H, CH₂—NH₂), 2.64 (m, 6H, 4H piperazine+CH₂-pip.) 3.05 (m, 4H, piperazine), 3.71 (t, 2H, CH₂-phthalimide), 3.83 (s, 3H, —OCH₃), 6.94 (m, 4H, H arom, arylpiperazine).

### Step 5: Preparation of 2H-chromene-3-carboxylic acid {4-[4-(2-methoxyphenyl)-piperazin-1-yl]-butyl}-amide

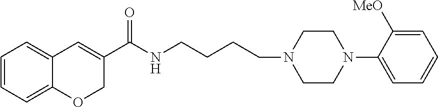

Dissolve successively 0.33 g of 2H-chromene-3-carboxylic acid (1.9 mmol) obtained in previous step 2 and 0.5 g of 4-[4-(2-methoxy phenyl)piperazin-1-yl]-butylamine (1.9 mmol) in 10 ml of dichloromethane. Add 0.5 ml of triethylamine (3.08 mmol) and 0.61 g of TBTU (1.9 mmol). The mixture is placed under agitation for 4 h. Adjust the organic volume to 25 ml then wash the phase twice with 25 ml of water. After drying and concentration, the organic residue is chromatographed on silica by using a suitable dichloromethane-ethyl acetate gradient. After purification, 2H-chromene-3-carboxylic acid {4-[4-(2-methoxyphenyl)-piperazin-1-yl]-butyl}-amide is obtained in the form of a thick brown oil with a yield of 70%. ¹H NMR (CDCl₃ base): 1.65-1.68 (m, 4H, —CH₂—CH₂—), 2.46 (t, 2H, —CH₂—N), 2.66 (m, 4H, H-piperazine), 3.09 (m, 4H, H-piperazine), 3.37-3.41 (m, 2H, —CH₂—N—CO—), 5.00 (s, 2H, O—CH₂), 6.50 (s, 1H, —NH), 6.83-7.26 (m, 9H, H arom+H₄).

Preparation of the salt: Dissolve 0.554 g of the base (1.31 mmol) in 10 ml of ethyl acetate. Add 0.83 ml of a 3.3 N solution of isopropanol-HCl (2.7 mmol). After concentration, take up the salt in ethyl ether, then filter and dry the salt. 2H-chromene-3-carboxylic acid {4-[4-(2-methoxyphenyl)piperazin-1-yl]-butyl}-amide dihydrochloride is isolated in the form of a cream colored powder with a yield of 74%. Analysis (salt): C₂₅H₃₁O₃N₃-2HCl Mass=421.54. MS (APCI⁺, 600° C.): MH⁺=422.2 (100%). MP=224° C.

### EXAMPLE 2

### 2H-Chromene-3-carboxylic acid {4-[4-(2,3-dichlorophenyl)-piperazin-1-yl]-butyl}-amide

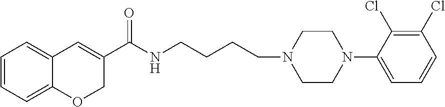

This compound is prepared according to the procedure of example 1, but using corresponding reagents. The acid used is 2H-chromene-3-carboxylic acid, obtained in step 2 of example 1; the amine used is prepared from 1-(2,3-dichlorophenyl)-piperazine, according to the same procedure as for obtaining 4-[4-(2-methoxy phenyl)-piperazin-1-yl]-butylamine in steps 3 and 4 of example 1. Thus, 2H-chromene-3-carboxylic acid {4-[4-(2,3-dichlorophenyl)-piperazin-1-yl]-butyl}-amide is obtained in the form of a yellow solid with a yield of 57%. ¹H NMR (CDCl₃): 1.63-1.68 (m, 4H, —CH₂—CH₂—), 2.48 (t, 2H, —CH₂—N), 2.65 (m, 4H, H-piperazine), 3.06 (m, 4H, H-piperazine), 3.37-3.42 (m, 2H, —CH₂—N—CO—), 5.00 (s, 2H, O—CH₂), 6.46 (s, 1H, —NH), 6.84-7.21 (m, 7H, H arom).

Preparation of the salt: Dissolve 0.434 g of the base (0.87 mmol) in 10 ml of ethyl acetate. Add 0.3 ml of a 3.3 N solution of isopropanol-HCl (1 mmol). After concentration, take up the salt in ethyl ether, filter and then dry the salt. 2H-chromene-3-carboxylic acid {4-[4-(2,3-dichlorophenyl)piperazin-1-yl]-butyl}-amide hydrochloride is isolated in the form of a cream-colored powder with a yield of 83%. Analysis (salt): C₂₄H₂₇O₂N₃Cl₂—HCl Mass=496.87. MS (ESI⁺, 250° C.): MH⁺=460.1 (100%). MP=201° C.

### EXAMPLE 3

### 2-H-Chromene-3-carboxylic acid {4-[4-(2-fluorophenyl)-piperazin-1-yl]-butyl}-amide

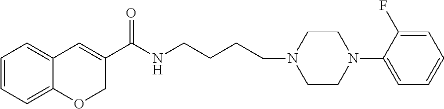

This derivative is obtained according to the procedure of example 1, but with corresponding reagents. ¹H NMR (CDCl₃ base): 1.63-1.68 (m, 4H, —CH₂—CH₂—), 2.48 (t, 2H, CH₂—N), 2.67 (m, 4H, H piperazine), 3.12 (m, 4H, H-piperazine), 3.37-3.42 (m, 2H, —CH₂—N—CO—), 5.00 (s, 2H, O—CH₂), 6.45 (s, 1H, —NH), 6.83-7.07 (m, 9H, H arom+H4). Analysis (sel): C₂₄H₂₈O₂FN₃—HCl Mass-445.97. MS (ESI⁺, 250° C.): MH⁺=410.3 (100%). MP=231° C.

### EXAMPLE 4

### 2H-Chromene-3-carboxylic acid {4-(4-phenylpiperazin-1-yl)-butyl}-amide

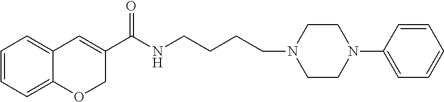

This derivative is obtained according to the procedure of example 1, but with corresponding reagents. ¹H NMR (CDCl₃ base): 1.62-1.66 (m, 4H, —CH₂—CH₂—), 2.45 (t, 2H, —CH₂—N), 2.60-2.63 (m, 4H, H-piperazine), 3.19-3.21 (m, 4H, H-piperazine), 3.40 (t, 2H, —CH₂—N—CO—), 5.00 (s, 2H, O—CH₂), 6.36 (s, 11, —NH), 6.71-7.27 (m, 10H, H arom+H4). Analysis (salt): C₂₄H₂₉O₂N₃—HCl Mass=427.98. MS (ESI⁺, 400° C.): MH⁺=392.3 (100%). MP=239° C.

### EXAMPLE 5

### 2H-Chromene-3-carboxylic acid {4-[4-(4-chlorophenyl)-piperazin-1-yl]-butyl}-amide

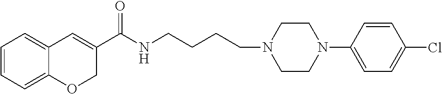

This derivative is obtained according to the procedure of example 1, but with corresponding reagents. ¹H NMR (CDCl₃ base): 1.63-1.67 (m, 4H, —CH₂—CH₂—), 2.44 (t, 2H, —CH₂—N), 2.59-2.61 (m, 4H, H piperazine), 3.14-3.17 (m, 4H, H piperazine), 3.40 (t, 2H, —CH₂—N—CO—), 4.99 (s, 2H, O—CH₂), 6.24 (s, 1H, —NH), 6.71-7.22 (m, 9H, H arom+H4). Analysis (salt): C₂₄H₂₈O₂ClN₃—HCl Mass=462.42. MS (ESI⁺, 400° C.): MH⁺=426.2 (100%). MP=236° C.

### EXAMPLE 6

### 2H-Chromene-3-carboxylic acid {4-[4-(3 chlorophenyl)-piperazin-1-yl]-butyl}-amide

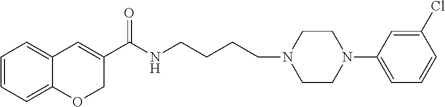

This derivative is obtained according to the procedure of example 1, but with corresponding reagents. ¹H NMR (CDCl₃ base): 1.66 (m, 4H, —CH₂—CH₂—), 2.44 (t, 2H, —CH₂—N), 2.56-2.60 (m, 4H, H piperazine), 3.18-3.21 (m, 4H, H-piperazine), 3.40 (t, 2H, —CH₂—N—CO—), 5.00 (s, 2H, O—CH₂), 6.20 (s, 1H, —NH), 6.73-7.20 (m, 9H, H arom+H4). Analysis (salt): C₂₄H₂₈O₂ClN₃—HCl Mass=462.42. MS (ESI⁺, 400° C.) MH⁺=426.2 (100%). MP=216° C.

### EXAMPLE 7

### 2H-Chromene-3 carboxylic acid {4-[4-(2-chlorophenyl)-piperazin-1-yl]-butyl}-amide

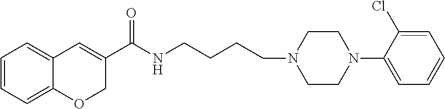

This derivative is obtained according to the procedure of example 1, but with corresponding reagents. ¹H NMR (CDCl₃ base): 1.65-1.67 (m, 4H, —CH₂—CH₂—), 2.47 (t, 2H, —CH₂—N), 2.65 (m, 4H, H piperazine), 3.07 (m, 4H, H-piperazine), 3.37-3.41 (m, 2H, —CH₂—N—CO—), 5.01 (s, 2H, O—CH₂), 6.49 (s, 1H, —NH), 6.84-7.35 (m, 9H, H arom+H4). Analysis (salt): C₂₄H₂₈O₂ClN₃—HCl Mass=462.42. MS (ESI⁺, 400° C.): MH⁺=426.2 (100%). MP=201° C.

### EXAMPLE 8

### 2H Chromene-3-carboxylic acid {4-[4-(4 fluorophenyl)-piperazin-1-yl]-butyl}-amide

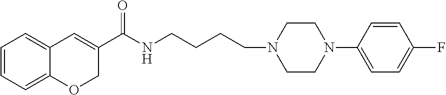

This derivative is obtained according to the procedure of example 1, but with corresponding reagents. ¹H NMR (CDCl₃ base): 1.62-1.66 (m, 4H, —CH₂—CH₂—), 2.45 (t, 2H, —CH₂—N), 2.60-2.62 (m, 4H, H-piperazine), 3.10-3.13 (m, 4H, H-piperazine), 3.37-3.40 (m, 2H, —CH₂—N—CO—), 5.00 (s, 2H, O—CH₂), 6.37 (s, 1H, —NH), 6.82-7.22 (m, 9H, H arom+H4). Analysis (salt): C₂₄H₂₈O₂FN₃—HCl Mass=445.97. MS (ESI⁺, 400° C.): MH⁺=410.2 (100%). MP=243° C.

### EXAMPLE 9

### 2H Chromene-3-carboxylic acid {4-[4-(2-methylphenyl)-piperazin-1-yl]-butyl}-amide

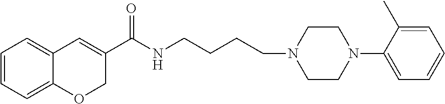

This derivative is obtained according to the procedure of example 1, but with corresponding reagents. ¹H NMR (CDCl₃ base): 1.63-1.67 (m, 4H, —CH₂—CH₂—), 2.29 (s, 3H, CH₃), 2.46 (t, 2H, —CH₂—N—), 2.61 (m, 4H, H-piperazine), 2.92-2.94 (m, 4H, H-piperazine), 3.39 (t, 2H, —CH₂—N—CO—), 5.01 (s, 2H, O—CH₂), 6.49 (s, 1H, —NH), 6.84-7.26 (m, 9H, H arom+H4). Analysis (salt): C₂₆H₃₃O₂N₃—HCl. Mass=442. MS (ESI⁺, 400° C.): MH⁺=406.3 (100%) MP=187° C.

### EXAMPLE 10

### 2H-Chromene-3-carboxylic acid {4-[4-(2,4-dimethylphenyl)-piperazin-1-yl]-butyl}-amide

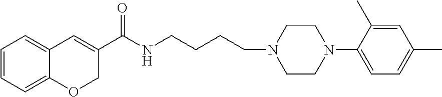

This derivative is obtained according to the procedure of example 1, but with corresponding reagents. ¹H NMR (CDCl₃ base): 1.64-1.68 (m, 4H, —CH₂—CH₂—), 2.26 (s, 6H, CH₃), 2.44 (t, 2H, —CH₂—N), 2.48 (m, 4H, H-piperazine), 2.89 (m, 4H, H-piperazine), 3.40 (t, 2H, —CH₂—N—CO—), 5.01 (s, 2H, O—CH₂), 6.48 (s, 1H, —NH), 6.84-7.20 (m, 8H, H arom+H4). Analysis (salt): C₂₆H₃₃O₂N₃—HCl Mass=456.03. MS (ESI⁺, 400° C.): MH⁺=420.3 (100%) MP=207° C.

### EXAMPLE 11

### 2H-Chromene-3-carboxylic acid {4-[4-(2,3-dimethylphenyl)piperazin-1-yl]-butyl}-amide

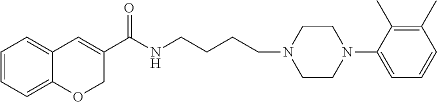

This derivative is obtained according to the procedure of example 1, but with corresponding reagents. ¹H NMR (CDCl₃ base): 1.63-1.67 (m, 4H, —CH₂—CH₂—), 2.21 (s, 3H, CH₃), 2.26 (s, 3H, CH₃), 2.44 (t, 2H, —CH₂—N), 2.48 (m, 4H, H-piperazine), 2.89 (m, 4H, H-piperazine), 3.39 (t, 2H, —CH₂—N—CO—), 5.01 (s, 2H, O—CH₂), 6.48 (s, 1H, —NH), 6.84-7.20 (m, 5H, H arom+H4) Analysis (salt): C₂₆H₃₃O₂N₃HCl, Mass=456.03. MS (ESI⁺, 400° C.): MH⁺=420.3 (100%). MP=202° C.

### EXAMPLE 12

### 2H-Chromene-3-carboxylic acid {4-[4-(2-cyanophenyl)-piperazin-1-yl]-butyl}-amide

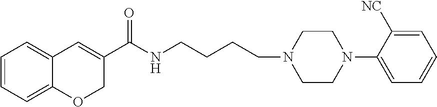

This derivative is obtained according to the procedure of example 1, but with corresponding reagents. ¹H NMR (CDCl₃ base): 1.65-1.66 (m, 4H, —CH₂—CH₂—), 2.50 (t, 2H, —CH₂—N), 2.68-2.70 (m, 4H, H-piperazine), 3.22-3.25 (m, 4H, H-piperazine), 3.39 (m, 2H, —CH₂—N—CO—), 5.00 (s, 2H, O—CH₂), 6.41 (s, 1H, —NH), 6.83-7.56 (m, 9H, H arom+H4). Analysis (salt): C₂₅H₂₈O₂N₄—HCl, Mass=452.99. MS (ESI⁺, 400° C.): MH⁺=417.3 (100%), MP=194° C.

### EXAMPLE 13

### 2H-Chromene-3-carboxylic acid {4-[4-(4-cyanophenyl)piperazin 1-yl]-butyl}-amide

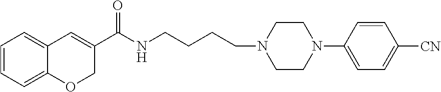

This derivative is obtained according to the procedure of example 1, but with corresponding reagents. ¹H NMR (CDCl₃ base): 1.64-1.67 (m, 4H, —CH₂—CH₂—), 2.44 (t, 2H, —CH₂—N), 2.57-2.59 (m, 4H, H-piperazine), 3.31-3.33 (m, 4H, H-piperazine), 3.4 (m, 2H, —CH₂—N—CO—), 4.99 (s, 2H, O—CH₂), 6.15 (s, 1H, —NH), 6.82-7.52 (m, 9H, H arom+H4). Analysis (salt): C₂₅H₂₈O₂N₄—HCl, Mass=452.99. MS (ESI⁺, 400° C.): MH⁺=417.3 (100%). MP=212° C.

### EXAMPLE 14

### 2H-Chromene-3-carboxylic acid {4-[4-(3-cyanophenyl)-piperazin-1-yl]-1-butyl}-amide

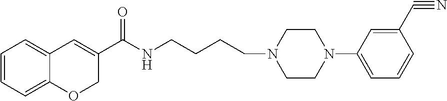

This compound is prepared according to the procedure of example 1, but using corresponding reagents. The acid used is 2H-chromene-3-carboxylic acid, obtained in step 2 of example 1; the amine used is prepared from 1-(3cyanophenyl)-piperazine according to the same procedure as for obtaining 4-[4-(2-methoxy-phenyl)-piperazin-1-yl]-butylamine in steps 3 and 4 of example 1. 2H-Chromene-3-carboxylic acid {4-[4-(3-cyanophenyl)-piperazin-1-yl]-butyl}-amide is obtained in the form of a beige solid with a yield of 72%. ¹H NMR (CDCl₃ base): 1.64-1.68 (m, 2H, —CH₂—), 2.03 (m, 2H, —CH2-), 2.47 (t, 2H, —CH₂—N), 2.61-2.64 (m, 4H, H-piperazine), 3.21-3.38 (m, 4H, H-piperazine), 3.38-3.42 (m, 2H, —CH₂—N—CO—), 4.99 (s, 2H, O—CH₂—), 6.32 (s, 1H, —NH), 6.84-7.32 (m, 9H, H arom+H₄).

Preparation of the salt: Dissolve 0.580 g of the base (1.4 mmol) in 10 ml of ethyl acetate. Add 0.44 ml of a 3.3 N solution of isopropanol-HCl (1 mmol). After concentration, take up the salt in ethyl ether, then filter and dry the salt. H-chromene-3-carboxylic acid {4-[4-(3-cyanophenyl)-piperazin-1-yl]-butyl}-amide hydrochloride is isolated in the form of a cream-colored powder with a yield of 83%. Analysis (salt): C₂₅H₂₈O₂N₄—HCl Mass=452.99. MS (ESI⁺, 250° C.): MH⁺=417.2 (100%). MP=229° C.

### EXAMPLE 15

### 2-H-Chromene-3-carboxylic acid {4-[4-(4-trifluoromethylphenyl)-piperazin-1-yl]-butyl}-amide

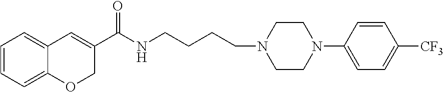

This derivative is obtained according to the procedure of example 1, but with corresponding reagents. ¹H NMR (CDCl₃ base): 1.65-1.67 (m, 4H, —CH₂—CH₂—), 2.50 (t, 2H, —CH₂—N), 2.59-2.61 (m, 4H, H-piperazine), 3.29-3.26 (m, 4H, H-piperazine), 3.42 (t, 2H, —CH₂—N—CO—), 4.99 (s, 2H, O—CH₂), 6.28 (s, 1H, —NH), 6.82-7.52 (m, 9H, H arom+H4). Analysis (salt): C₂₅H₂₈O₂N₃F₃—HCl Mass=495.98. MS (ESI⁺, 400° C.): MH⁺=460.3 (100%). MP=261.9° C.

### EXAMPLE 16

### 2H-Chromene-3-carboxylic acid {4-[4-(3-trifluoromethylphenyl)-piperazin-1-yl]-butyl}-amide

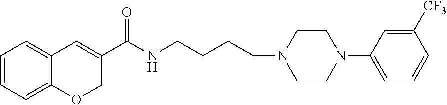

This derivative is obtained according to the procedure of example 1, but with corresponding reagents. ¹H NMR (CDCl₃ base): 1.66 (m, 4H, —CH₂—CH₂—), 2.44 (t, 2H, —CH₂—N), 2.56-2.60 (m, 4H, H-piperazine), 3.18-3.21 (m, 4H, H-piperazine), 3.41 (t, 2H, —CH₂—N—CO—), 5.00 (s, 2H, O—CH₂), 6.29 (s, 1H, —NH), 6.83-7.33 (m, 9H, H arom+H4). Analysis (salt): C₂₅H₂₈O₂F₃N₃—HCl, Mass=495.98. MS (ESI⁺, 400° C.): MH⁺=460.3 (100%). MP=234° C.

### EXAMPLE 17

### 2H-Chromene-3 carboxylic acid {4-[4-(2-trifluoromethylphenyl)-piperazin-1-yl]-butyl}-amide

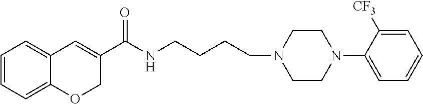

This derivative is obtained according to the procedure of example 1, but with corresponding reagents. ¹H NMR (CDCl₃ base): 1.65-1.67 (m, 4H, —CH₂—CH₂—), 2.50 (t, 2H, —CH₂—N), 2.66 (m, 4H, H-piperazine), 2.97 (m, 4H, H-piperazine), 3.40 (t, 2H, —CH₂—N—CO—), 5.02 (s, 2H, O—CH₂), 6.61 (s, 1H, —NH), 6.83-7.62 (m, 9H, H arom+H4). Analysis (salt): C₂₅H₂₈O₂N₃F₃—HCl. Mass=495.98. MS (ESI⁺, 400° C.): MH⁺=460.3 (100%). MP=161° C.

### EXAMPLE 18

### 2H-Chromene 3 carboxylic acid {4-[4-(4-nitrophenyl)-piperazin-1-yl]-butyl}-amide

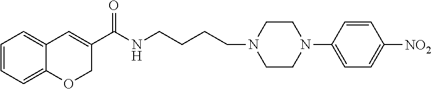

This derivative is obtained according to the procedure of example 1, but with corresponding reagents. ¹H NMR (CDCl₃ base) 1.64-1.65 (m, 4H, —CH₂—CH₂—), 2.43-2.45 (t, 2H, —CH₂—N), 2.58-2.66 (m, 4H, H-piperazine), 3.38-3.43 (m, 4H, H-piperazine), 3.55 (m, 2 Hr CH₂—N—CO—), 5.00 (s, 2H, O—CH₂), 6.17 (s, 1H, —NH), 6.71-8.12 (m, 9 Hr H arom+H₄). Analysis (salt): C₂₄H₂₈O₄N₄—HCl. Mass=472.98. MS (ESI⁺, 400° C.): MH⁺=437.2 (100%). MP=243° C.

In an analogous manner the following compounds are obtained using corresponding reagents:

### EXAMPLE 19

### 2H Chromene 3 carboxylic acid {4-[4-(3-nitrophenyl)-piperazin-1-yl]-butyl}-amide

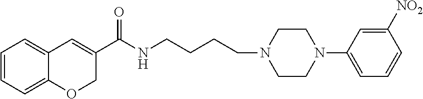

Analysis: C₂₄H₂₈N₄O₄, MW=436.52

### EXAMPLE 20

### 2H-Chromene-3-carboxylic acid {4-[4-(3-nitrophenyl)piperazin-1-yl]-butyl}-amide

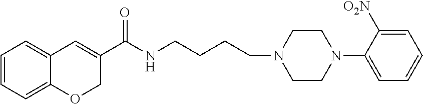

Analysis: C₂₄H₂₈N₄O₄, MW=436.52

### EXAMPLE 21

### 3-(4-{4-[(2H-Chromene-3-carbonyl)-amino]-butyl}-piperazin-1-yl)-benzoic acid ethyl ester hydrochloride

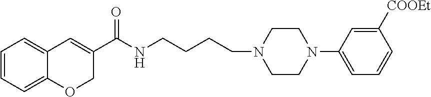

¹H NMR (CDCl₃ base): 1.39 (t, 3H, —O—CH₂—CH₃), 1.65-1.66 (m, 4H, —CH₂—CH₂—), 2.46 (t, 2H, —CH₂—N), 2.62-2.64 (m, 4H, H-piperazine), 3.24-3.27 (m, 4H, H-piperazine), 3.38-3.41 (t, 2H, —CH₂—N—CO—), 4.36 (q, 2H, O—CH₂—CH₃), 5.00 (s, 2H, O—CH₂), 6.33 (s, 1H, —NH), 6.78-7.58 (m, 9H, H arom+H4). Analysis (salt): C₂₇H₃₃O₄N₃—HCl, Mass=500.04, MS (ESI⁺, 400° C.): MH⁺=464.3 (100%). MP=229° C.

### EXAMPLE 22

### 4-(4-{4-[(2H-Chromene-3-carbonyl)-amino]-butyl}-piperazin-1-yl)-benzoic acid ethyl ester hydrochloride

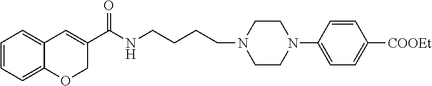

¹H NMR (CDCl₃ base): 1.37 (t, 3H, —O—CH₂—CH₃), 1.67 (m, 4H, —CH₂—CH₂), 2.44 (t, 2H, —CH₂—N), 2.58-2.60 (m, 4H, H-piperazine), 3.31-3.34 (m, 4H, H piperazine), 3.37-3.42 (t, 2H, —CH₂—N—CO—), 4.34 (q, 2H, —O—CH₂—CH₃), 5.00 (s, 2H, O—CH₂), 6.27 (s, 1H, —NH), 6.64-7.92 (m, 9H, Haro+H4). Analysis (salt): C₂₇H₃₃O₄N₃—HCl, Mass=500.04. MS (ESI⁺, 400° C.): MH⁺=464.3 (100%). MP=237° C.

### EXAMPLE 23

### 2H-Chromene 3 carboxylic acid {4-[4-(3,5-dimethoxyphenyl)piperazin-1-yl]-butyl}-amide hydrochloride

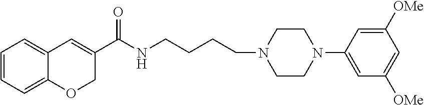

¹H NMR (CDCl₃ base): 1.66 (m, 4H, —CH₂—CH₂—), 2.46-2.48 (t, 2H, —CH₂—N), 2.62-2.65 (m, 4H, H-piperazine), 3.19-3.22 (m, 4H, H-piperazine), 3.39 (t, 2H, —CH₂—N—CO—), 3.77 (s, 6H, —OCH₃), 5.00 (s, 2H, O—CH₂), 6.37 (s, 1H, —NH), 6.88-7.26 (m, 9H, H arom+H4). Analysis (salt): C₂₅H₂₈O₂N₃F₃—HCl, Mass=495.9B. MS (ESI⁺, 400° C.): MH⁺=452.3 (100%). MP=213° C.

In an analogous manner the following compounds are obtained from corresponding reagents:

### EXAMPLE 24

### 2H-Chromene-3-carboxylic acid {4-[4-(3-methoxyphenyl)-piperazin-1-yl]-butyl}-amide hydrochloride

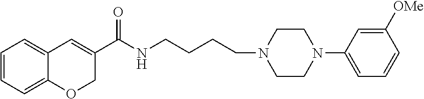

Analysis: C₂₅H₃₁N₃O₃, MW=421.54

### EXAMPLE 25

### 2H-Chromene-3-carboxylic acid {4-[4-(4-methoxyphenyl)-piperazin-1-yl]-butyl}-amide hydrochloride

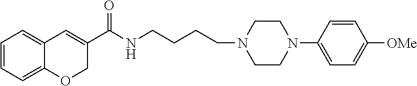

Analysis: C₂₅H₃₁N₃O₃, MW=421.54

### EXAMPLE 26

### 2H-Chromene-3-carboxylic acid {4-[4-(3,4-dimethoxyphenyl)-piperazin-1-yl]-butyl}-amide hydrochloride

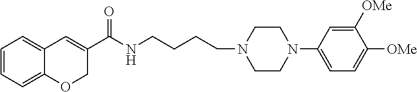

Analysis: C₂₆H₃₃N₃O₄, MW=451.57

### EXAMPLE 27

### 2H-Chromene-3-carboxylic acid {4-[4-(3,4-methylenedioxyphenyl)-piperazin-1-yl]-butyl}-amide hydrochloride

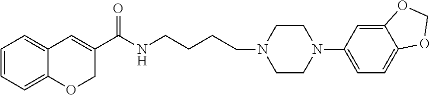

Analysis: C₂₅H₂₉N₃O₄, MW=435.53

### EXAMPLE 28

### 6-Chloro-2H-Chromene-3-carboxylic acid {4-[4-(2,3-dichlorophenyl)-piperazin-1-yl]-butyl}-amide

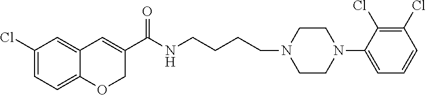

### Step 1: Preparation of 6-chloro-2H-chromene-3-carbonitrile

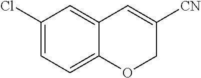

According to the same procedure as in step 1 of example 1, 6-chloro-2H-chromene-3-carbonitrile is prepared. Heat at 80° C. 10 g of 5-chlorosalicylic aldehyde (0.064 mol) diluted in 17 g of acrylonitrile (0.32 mol) and then add to the mixture 1.6 g of DABCO (0.015 mol). After 8 h of heating, the reaction mixture is allowed to return to room temperature. Add 100 ml of 1 N NaOH, extract three times in dichloromethane, dry the organic phase on MqSO₄, filter and concentrate under a vacuum. The solid obtained is chromatographed on silica (eluent: dichloromethane) to give 6.1 g of 6-chloro-2H-chromene 3 carbonitrile in the form of a yellow powder with a yield of 50%. ¹H NMR (DMSO): 4.92 (s, 2H, O—CH₂—) 6.94 (d, 1H, H arom), 7.31-7.39 (m, 2H, H arom), 7.55 (s, 1H, H4).

### Step 2: Nitrile hydrolysis

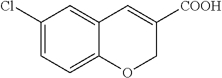

Hydrolysis of the 6 chloro-2H-chromene-3-carbonitrile obtained in the previous step leads, by a method identical to that described in example 1, in step 2 to 6 chloro-2H-chromene 3 carboxylic acid obtained in the form of a yellow powder with a yield of 94%, which is used directly in the following step. ¹H NMR (DMSO): 4.93 (s, 2H, O—CH₂—), 6.87 (d, 1H, H arom), 7.28 (dd, 1H, H arom), 7.43 (s, 1H, H₄), 7.45 (d, 1H, Haro), 13.01 (m, 1H, COOH).

### Step 3: Preparation of 6-chloro-2H-chromene-3-carboxylic acid {4-[4-(2,3-dichlorophenyl)-piperazin-1-yl]-butyl}-amide

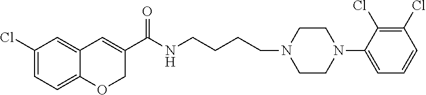

This compound is prepared according to the procedure of example 1, but using corresponding reagents. The acid used is 6-chloro-2H-chromene-3-carboxylic acid, obtained in previous step 2; the amine used is prepared from 1-(2,3-dichlorophenyl)-piperazine according to the same procedure as for obtaining 4-[4-(2-methoxy-phenyl)piperazin-1-yl]-butylaminein in steps 3 and 4 of example 1. 6 chloro 2H chromene-3-carboxylic acid {4-[4-(2,3-dichlorophenyl)-piperazin-1-yl]-butyl}-amide is obtained in the form of a yellow solid with a yield of 54%. ¹H NMR (CDCl₃ base): 1.65-1.69 (m, 4H, —CH₂—CH₂—), 2.47 (t, 2H, —CH₂—N), 2.65 (m, 4H, H-piperazine), 3.06 (m, 4H, H-piperazine), 3.33-3.42 (m, 2H, —CH₂—N—CO—), 5.00 (s, 2H, O—CH₂—), 6.52 (s, 1H, —NH), 6.77-7.17 (m, 7H, H arom+H4).

Preparation of the salt: Dissolve 0.464 g of the base (0.94 mmol) in 10 ml of ethyl acetate. Add 0.33 ml of a 3.3 N solution of isopropanol-HCl (1 mmol). After concentration, take up the salt in ethyl ether, then filter and dry the salt. 6-chloro-2H-chromene-3-carboxylic acid {4-[4-(2,3-dichlorophenyl)-piperazin-1-yl]-butyl}-amide hydrochloride is isolated in the form of a cream-colored powder with a yield of 83%. Analysis (salt): C₂₄H₂₆O₂N₃Cl₃—HCl Mass=531.31. MS (ESI⁺, 250° C.): MH⁺=496.1 (100%). MP=214° C.

### EXAMPLE 29

### 6-Chloro-2H-chromene-3-carboxylic acid {4-[4-(2-methoxyphenyl)-piperazin-1-yl]-butyl}-amide

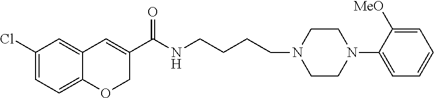

This derivative is obtained according to the procedure of example 1, but with corresponding reagents. ¹H NMR (CDCl₃ base): 1.63-1.66 (m, 4H, —CH₂—CH₂—), 2.46 (t, 2H, —CH₂—N), 2.66 (m, 4H, H piperazine), 3.09 (m, 4H, H-piperazine), 3.38 (m, 2H, CH₂—N—CO—), 3.75 (s, 3H, OCH₃), 4.90 (s, 2H, O—CH₂), 6.62 (s, 1H, —NH), 6.77-7.15 (m, 8H, H arom+H4). Analysis (salt): C₂₅H₃₀O₃N₃Cl—HCl. Mass=492.45. MS (ESI⁺=400° C.): MH⁺=456.2 (100%). MP=155° C.

### EXAMPLE 30

### 6-Chloro-2H-chromene-3-carboxylic acid {4-[4-(2-fluorophenyl)-piperazin-1-yl]-butyl}-amide

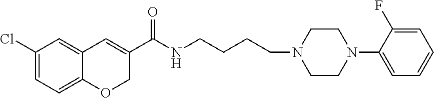

This derivative is obtained according to the procedure of example 1, but with corresponding reagents. ¹H NMR (CDCl₃ base): 1.61-1.66 (m, 4H, —CH₂—CH₂—), 2.45 (t, 2H, —CH₂—N), 2.63-2.65 (m, 4H, H-piperazine), 3.09-3.12 (m, 4H, H piperazine), 3.37-3.41 (m, 2H, —CH₂—N—CO—), 5.00 (s, 2H, O—CH₂), 6.49 (s, 1H, NH), 6.77-7.15 (m, 8H, H arom+H₄). Analysis (salt) C₂₄H₂₇O₂N₃ClF—HCl. Mass=480.41. MS (ESI⁺, 250° C.: MH⁺=444.2 (100%). MP=214° C.

### EXAMPLE 31

### 6-Chloro-2H-chromene-3-carboxylic acid {4-[4-(3-cyanophenyl)-piperazin-1-yl]-butyl}-amide

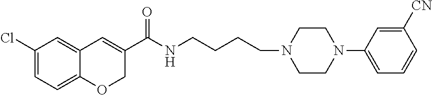

This derivative is obtained according to the procedure of example 1, but with corresponding reagents. ¹H NMR (CDCl₃ base): 1.63-1.67 (m, 4H, —CH₂—CH₂—), 2.43 (t, 2H, —CH₂—N), 2.59-2.61 (m, 4H, H-piperazine), 3.20-3.23 (m, 4H, H-piperazine), 3.37-3.42 (m, 2H, —CH₂—N—CO—), 4.99 (s, 2H, O—CH₂), 6.56 (s, 1H, —NH), 6.76-7.40 (m, 8H, H arom+H₄). Analysis (salt): C₂₅H₂₇O₂N₄Cl—HCl. Mass=487.43. MS (APCI⁺, 500° C.) MH⁺=451.2 (100%). MP=120° C. decom.

### EXAMPLE 32

### 2H-thiochromene-3-carboxylic acid {4-[4-(2,3-dichlorophenyl)piperazin-1-yl]-butyl}-amide

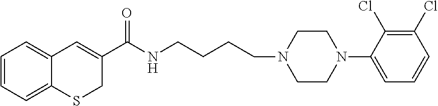

### Step 1: 2,2′-Dithiodibenzaldehyde

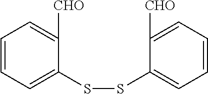

The procedure used is that described in Synthesis 1989, 763. Into a 1 liter round bottomed flask under nitrogen, introduce 5 g of 2-mercaptobenzyl alcohol (0.035 mol. 1 eq) and 350 ml of dry toluene. Then add 46 q of MnO₂ (0.53 mol. 15 eq) and carry the mixture at 40° C. for 5 h. After returning to room temperature, filter the toluene mixture on silica then elute with a 50/50 n-heptane/dichloromethane mixture. 3.5 g of 2,2′-dithiodibenzaldehyde is recovered in the form of a white solid (yield=70%). ¹H NMR (CDCl₃): 7.37-7.41 (m, 2H), 7.47-7.51 (m, 2H), 7.77-7.79 (m, 2H), 7.86-7.88 (m, 2H), 10.23 (s, 2H, CHO).

### Step 2: 2H-thiochromene-3-carbonitrile

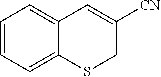

The procedure used is that described in Synthesis 2001, 2389. In a 250 ml round-bottomed flask, introduce 3.5 g of 2,2′-dithiodibenzaldehyde (0.013 mol, 1 eq) obtained in previous step 1, add 13 ml of acrylonitrile (0.197 mol, 15 eq) and 3 ml of DBU (0.02 mol, 1.5 eq) dropwise. The mixture becomes homogeneous and orange in color. After one night at room temperature, chromatograph the mixture directly and elute with 40/60 n-heptane/dichloromethane. 4.24 g of 2H-thiochromene-3-carbonitrile is recovered in the form of a yellow solid (yield=90%), which is used in the following step. ¹H NMR (DMSO-d₆): 3.76 (d, 2H, J_{HH}=0.8 Hz, SCH₂), 7.20-7.38 (m, 4H, H arom), 7.54 (s, 1H, CH═).

### Step 3: Preparation de l'acide 2H-thiochromene-3-carboxylique

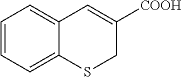

In a 250 ml round-bottomed flask, introduce 1.23 g of 2H-thiochromene-3-carbonitrile (0.007 mol) obtained in previous step 2 and 22 ml of 10% NaOH. Heat the mixture at 100-110° C. for 3 h. Allow to return to room temperature and then extract with dichloromethane. The aqueous phase is then acidified and extracted again with dichloromethane. The organic phase is dried on Na₂SO₄, filtered and evaporated. 1 g of 2H-thiochromene-3-carboxylic acid is recovered in the form of a yellow solid (yield=73%), which is used directly in the following step. ¹H NMR (CDCl₃) 3.75 (s, 2H, SCH₂), 7.12-7.33 (m, 4H, H arom), 7.67 (s, 1H, CH═).

### Step 4: Preparation of 2H-thiochromene-3-carboxylic acid {4-[4-(2,3-dichlorophenyl)-piperazin-1-yl]-butyl}-amide

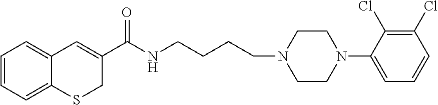

This compound is prepared according to the procedure for the 2H-chromene-3-carboxylic acid {4-[4-(2-methoxyphenyl)-piperazin-1-yl]-butyl}-amide obtained in example 1, but using the 2H-thiochromene-3-carboxylic acid obtained in previous step 3. 0.7 g of 2H-thiochromene-3-carboxylic acid {4-[4-(2,3-dichlorophenyl)-piperazin-1-yl]-butyl}-amide is recovered (yield=73%). ¹H NMR (DMSO): 1.45-1.55 (m, 4H, CH₂), 2.35-2.38 (m, 2 Hr CH₂N), 2.51-2.54 (m, 4H, CH₂ piperazine), 2.97-3.02 (m, 4H, CH₂ piperazine), 3.18-3.23 (m, 2H, CH₂NHCO), 3.67 (s, 2 Hr CH₂S), 7.10-7.30 (m, 8H, H arom and CH═), 8.26 (t, 1H, NHCO).

Preparation of the salt: Dissolve 0.68 g of the base (1.47 mmol, 1 eq) in 5 ml of dichloromethane. Introduce 0.94 ml of a 3.3 N solution of HCl in isopropanol (2.1 eq). Evaporate and take up in ethyl ether. Filter and dry the salt formed. Yield: 90%, MP=215° C., ¹H NMR (DMSO): 1.50-1.58 (m, 2H, CH₂), 1.76-1.81 (m, 2H, CH₂), 3.15-3.27 (m, 8H, CH₂), 3.42-3.45 (m, 2H, CH₂), 3.56-3.59 (m, 2H, CH₂), 3.69 (s, 2H, CH₂S), 7.17-7.39 (m, OH, H arom and CH═), 8.40 (t, 1H, NHCO). MS (ESI, 400° C.): MH⁺=476.1 (100%); M+2H⁺=478.1 (62%).

### EXAMPLE 33

### 2H-Thiochromene-3-carboxylic acid {4-[4-(2-fluorophenyl)piperazin-1-yl]-butyl}-amide

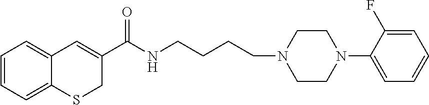

This compound is prepared according to the procedure for the 2H-chromene-3-carboxylic acid {4-[4-(2-methoxyphenyl)piperazin-1-yl]-butyl}-amide of example 1, but using corresponding reagents. 0.62 g of 2H-thiochromene-3-carboxylic acid {4-[4-(2-fluorophenyl)-piperazin-1-yl]-butyl}-amide is recovered (yield=73%). ¹H NMR (DMSO): 1.48-1.51 (m, 4H, CH₂), 2.33-2.36 (m, 2H, CH₂N), vers 2.50 cache par le pic du DMSO (4H, CH₂ piperazine), 2.99-3.01 (m, 4H, CH₂ piperazine), 3.18-3.23 (m, 2H, CH₂NHCO), 3.67 (s, 2H, CH₂S), 6.95-7.30 (m, 9H, H arom and CH═), 8.25 (t, 1H, NHCO).

Preparation of the salt: Dissolve 0.62 g of the base (1.4 mmol, 1 eq) in 5 ml of dichloromethane. Introduce 0.9 ml of a 3.3 N solution of HCl in isopropanol (2.2 eq). Evaporate and take up in ethyl ether. Filter and dry the salt formed. Yield: 90%. MP=212° C. ¹H NMR (DMSO): 1.51-1.58 (m, 2H, CH₂), 1.76-1.83 (m, 2H, CH₂), 3.13-3.26 (m, 8H, CH₂), 3.47-3.59 (m, 4H, CH₂), 3.70 (s, 2H, CH₂S), 7.04-7.34 (m, 9H, H arom and CH═), 8.44 (t, 1H, NHCO), 11.06 (s, 1H, HCl). MS (ESI, 400° C.): MH⁺=426 (100%).

### EXAMPLE 34

### 2H-Thiochromene-3-carboxylic acid {4-[4-(2-methoxyphenyl)piperazin-1-yl]-butyl}-amide

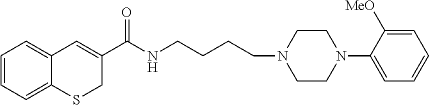

This derivative is obtained according to the procedure of example 1, but with corresponding reagents. ¹H NMR(CDCl₃ base): 1.67-1.75 (m, 4H, —CH₂—CH₂—), 2.45 (t, 2H, —CH₂—N), 2.65 (m, 4H, H-piperazine), 3.06 (m, 4H, H-piperazine), 3.40 (m, 2H, —CH₂—N—CO—), 3.70 (s, 2H, S—CH₂), 3.85 (s, 3H, O—CH₃), 6.68 (s, 1H, —NH), 6.79-7.28 (m, 9H, H arom+H₄). Analysis (salt): C₂₅H₃₁O₂N₃S—HCl Mass=474.07. MS (APCI⁺, 600° C.): MH⁺=438.2 (100%). MP=208° C.

### EXAMPLE 35

### 2H-Thiochromene-3-carboxylic acid {4-[4-(3-cyanophenyl)-piperazin-1-yl]-butyl}-amide

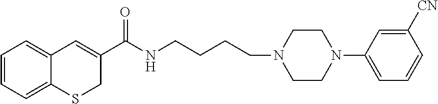

This compound is prepared according to the procedure for the 2H-chromene-3-carboxylic acid {4-[4-(2-methoxyphenyl)-piperazin-1-yl]-butyl}-amide of example 1, but using corresponding reagents. 0.61 g of 2H thiochromene-3-carboxylic acid {4-[4-(3-cyanophenyl)-piperazin-1-yl]-butyl}-amide is recovered (yield=70%). ¹H NMR (DMSO): 1.48-1.55 (m, 4H, CH₂), 2.32-2.35 (m, 2H, CH₂N), 2.47-2.50 (m, 4H, CH₂ piperazine), 3.16-3.22 (m, 6H, 2 CH₂ and CH₂NHCO), 3.67 (s, 2H, CH₂S), 7.13-7.39 (m, 9H, H arom), 8.26 (t, 1H, NHCO).

Preparation of the salt: Dissolve 0.61 g of the base (1.4 mmol, 1 eq) in 5 ml of dichloromethane. Introduce 0.9 ml of a 3.3 N solution of HCl in isopropanol (2.1 eq). Evaporate and take up in ethyl ether. Filter and dry the salt formed. Yield: 70%. MP=209° C. ¹H NMR (DMSO): 1.50-1.60 (m, 2H, CH₂), 1.73-1.77 (m, 2H, CH₂), 3.10-3.19 (m, 6H, CH₂), 3.21-3.26 (m, 2H, CH₂), 3.54-3.58 (m, 2H, CH₂), 3.69 (s, 2H, CH₂S), 3.95-3.98 (m, 2H, CH₂), 7.17-7.45 (m, 9H, H arom and CH═), 8.36 (t, 1H, NHCO), 10.22 (s, 1H, HCl). MS (ESI, 400° C.): MH⁺=433.2 (100%).

In an analogous manner, the following compounds are obtained from corresponding reagents:

### EXAMPLE 36

### 2H-Thiochromene-3-carboxylic acid {4-[4-(4-cyanophenyl)-piperazin-1-yl]-butyl}-amide

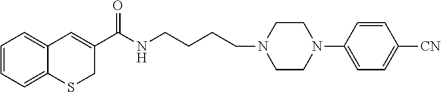

Analysis: C₂₅H₂₉N₄OS. MW=432.59

### EXAMPLE 37

### 2H-Thiochromene-3-carboxylic acid {4-[4-(2-cyanophenyl)-piperazin-1-yl]-butyl}-amide

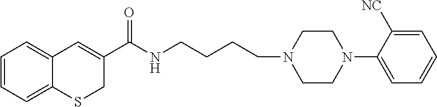

Analysis: C₂₅H₂₈N₄OS. MW=432.59

### EXAMPLE 38

### 2H-Thiochromene 3 carboxylic acid {4-[4-(3-methoxy phenyl)-piperazin-1-yl]-butyl}-amide

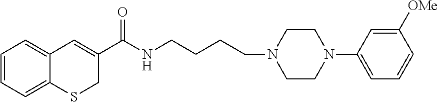

Analysis: C₂₅H₃₁N₃O₂S. MW=437.61

### EXAMPLE 39

### 2H-Thiochromene-3-carboxylic acid {4-[4-(4-methoxyphenyl)-piperazin-1-yl]-butyl}-amide

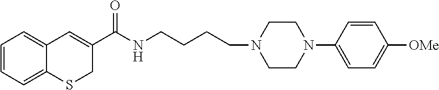

Analysis: C₂₋₅H₃₁N₃O₂S. MW=437.61

### EXAMPLE 40

### 2H-Thiochromene-3-carboxylic acid {4-[4-(3,4-dimethoxyphenyl)-piperazin-1-yl]-butyl}-amide

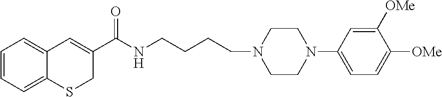

Analysis: C₂₆H₃₃N₃O₃S, MW=467.64.

### EXAMPLE 41

### 2H-Thiochromene-3-carboxylic acid {4-[4-(3-hydroxyphenyl)-piperazin-1-yl]-butyl}-amide

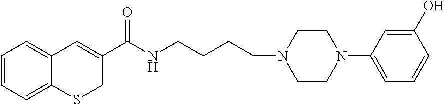

### Step 1: Preparation of 2H-thiochromene-3-carboxylic acid (4-hydroxybutyl)-amide

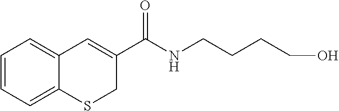

In a 250 ml round-bottomed flask, under nitrogen, introduce 2 q of 2H thiochromene-3-carboxylic acid (0.010 mole, 1 eq), 150 ml of dry dichloromethane and 15 drops of dry dimethylformamide. Cool the mixture to 0° C. and add dropwise 1.07 ml of oxalyl chloride (12 mmol, 1.2 eq). The mixture is then left at room temperature for 2 h, evaporated, then put back in solution in 15 ml of dry dichloromethane and added to a solution of 1.02 ml 4-amino-1-butanol (11 mmol, 1.1 eq) and 4.2 ml triethylamine (30 mmol, 3 eq) in 30 ml of dry dichloromethane. After one night of agitation at room temperature, the mixture is concentrated, purified on silica and eluted with a 90/10 dichloromethane/acetone mixture. 2.1 g of 2H-thiochromene-3-carboxylic acid {4-hydroxy-butyl}-amide is recovered in the form of a yellow solid (yield: 80%), which is used in the following step. ¹H NMR (DMSO d₆): 1.41-1.55 (m, 4H, CH₂), 3.16-3.19 (m, 2H, CH₂N), 3.39-3.44 (m, 2H, CH₂O), 3.67 (s, 2H, CH₂S), 4.41 (t, 1H, OH), 7.16-7.30 (m, 5H, H arom and CH═), 8.24 (t, 1H, NHCO).

### Step 2: Preparation of 2H-thiochromene-3-carboxylic acid (4-iodo-butyl)-amide

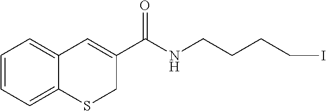

In a 100 ml round bottomed flask under nitrogen, introduce 1.65 g of triphenylphosphine (6.3 mmol, 1 eq), 0.43 q of imidazole (6.3 mmol, 1 eq) and 25 ml of dry dichloromethane. Add 1.76 g of iodine (6.9 mmol, 1.1 eq). A precipitate forms and the mixture becomes orange. After 5 min, add 1.66 g of the 2H-thiochromene-3-carboxylic acid {4-hydroxy-butyl}-amide (6.3 mmol, 1 eq) obtained in previous step 1, in solution in 25 ml of dichloromethane. After 4 h at room temperature, the reaction mixture is evaporated and purified by chromatography on silica and eluted with 70/30 n-heptane/ethyl acetate. 1.7 g of 2H-thiochromene-3-carboxylic acid (4-iodo-butyl)-amide is obtained in the form of an orange solid (yield: 72%). ¹H NMR (DMSO-d₆): 1.50-1.61 (m, 2H, CH₂), 1.74-1.84 (m, 2H, CH₂), 3.18-3.23 (m, 2H, CH₂), 3.26-3.33 (m, 2H, CH₂), 3.67 (s, 2H, CH₂S), 7.15-7.30 (m, 5H, H arom and CH═), 8.29 (t, 1H, NHCO).

### Step 3: Preparation of 2H-thiochromene-3-carboxylic acid {4-[4-(3-hydroxyphenyl)-piperazin-1-yl]-butyl}-amide

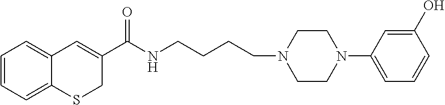

In a 250 ml round-bottomed flask, introduce 0.35 g 3-hydroxy phenyl piperazine (2 mmol, 1.1 eq), 0.33 g of K₂CO₃ (24 mmol, 1.3 eq) and 20 ml of acetonitrile. Add 0.7 g of the 2H-thiochromene-3-carboxylic acid {4-iodo-butyl}-amide (1.9 mmol, 1 eq) obtained in previous step 2, in solution in 10 ml of acetonitrile. Carry the mixture at reflux for 6 h. After returning to room temperature, the mixture is evaporated, taken up in dichloromethane and washed with water. The organic phase is then dried on Na₂SO₄, filtered, evaporated and purified by chromatography on silica and eluted with 95/5 dichloromethane/methanol. 0.4 g of 2H-thiochromene-3-carboxylic acid {4-[4-(3-hydroxyphenyl)piperazin-1-yl]-butyl}-amide is recovered in the form of a white solid (yield: 50%). ¹H NMR (DMSO): 1.50 (m, 4H, CH₂), 2.30-2.34 (m, 2H, CH₂N), 2.46-2.50 (m, 4H, CH₂ piperazine), 3.04-3.06 (m, 4H, CH₂ piperazine), 3.18-3.23 (m, 2H, CH₂NHCO), 3.67 (s, 2H, CH₂S), 6.19 (d, 1H, H arom), 6.28 (s, 1H, H arom), 6.35 (d, 1H, H arom), 6.96 (t, 1H, H arom), 7.15-7.30 (m, 5H, H arom, and CH═), 8.25 (t, 1H, NHCO), 9.08 (s, in, OH).

Preparation of the salt: Dissolve 0.4 g of the base (0.94 mmol, 1 eq) in 5 ml of methanol. Introduce 0.4 ml of a 5 N solution of HCl in isopropanol (2 eq). Evaporate and take up in ethyl ether. Filter and dry the salt formed. Yield: 57%. MP=182-184° C. ¹H NMR (DMSO): 1.51-1.58 (m, 2H, CH₂), 1.72-1.80 (m, 2H, CH₂), 2.99-3.17 (m, 6H, CH₂), 3.21-3.26 (m, 2H, CH₂), 3.52-3.54 (m, 2H, CH₂), 3.69 (s, 2H, CH₂S), 3.71-3.74 (m, 2H, CH₂), 6.30 (dd, 1H, H arom), 6.36 (s, 1H, H arom), 6.42 (d, 1H, H arom), 7.03 (t, 1H, H arom), 7.16-7.32 (m, 5H, H arom and CH═), 8.39 (t, 1H, NHCO), 9.27 (s large, 1H, OH), 10.47 (s, 1H, HCl). MS (APCI⁺, 150° C.): MH⁺=424.2 (100%).

In an analogous manner, the following compounds are obtained from corresponding reagents:

### EXAMPLE 42

### 2H-Thiochromene-3-carboxylic acid {4-[4-(2-hydroxyphenyl)-piperazin-1-yl]-butyl}-amide

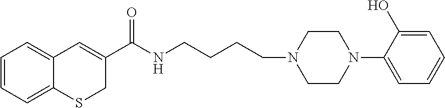

Analysis: C₂₄H₂₉N₃O₂S, MW=423.58.

### EXAMPLE 43

### 2H-Thiochromene-3-carboxylic acid {4-[4-(4-hydroxyphenyl)piperazin-1-yl]-butyl}-amide

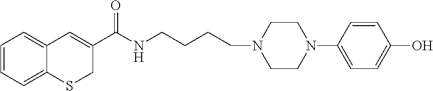

Analysis: C₂₄H₂₉N₃O₂S, MW=423.58.

### EXAMPLE 44

### 2,2-Dimethyl-2H thiochromene-3-carboxylic acid {4-[4-(3-cyanophenyl)-piperazin-1-yl]-butyl}-amide

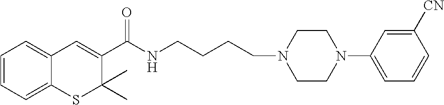

### Step 1: Preparation of 2,2-dimethyl-2H-thiochromene-3-carbonitrile

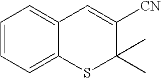

The procedure used is that described in Synthesis 2001, 2389. In a 500 ml round-bottomed flask, introduce 7 g of the 2,2′-dithiodibenzaldehyde (25 mmol, 1 eq) obtained in step 1 of example 28, add 40 ml of dimethylacrylonitrile (0.38 mole, 15 eq) and dropwise 6 ml of DBU (38 mmol, 1.5 eq). Heat at 100° C. for 5 h, then after returning to room temperature chromatograph the mixture directly and elute with 50/50 n-heptane/dichloromethane. 5 g of 2,2-dimethyl-2H-thiochromene-3-carbonitrile is recovered (yield: 50%). ¹H NMR (DMSO): 1.50 (s, 6H, 2 CH₃), 7.23-7.27 (m, 1H, H arom), 7.33-7.36 (m, 2H, H arom), 7.47 (d, 1H, H arom), 7.57 (s, 1H, CH═).

### Step 2: Preparation of 2,2-dimethyl-2H-thiochromene-3-carboxylic acid

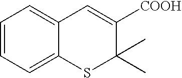

In a 100 ml round bottomed flask, introduce 0.5 g of the 2,2-dimethyl-2H-thiochromene-3-carbonitrile (2.5 mmol, 1 eq) obtained in previous step 1, add 10 ml of an aqueous solution saturated with KOH and methanol to complete solubilization of the mixture. Heat at 100° C. for 8 h. After returning to room temperature, add ice and acidify with concentrated HCl. Extract with ethyl acetate. The organic phase is dried on Na₂SO₄₁ filtered, evaporated and purified on silica and eluted with 95/5 dichloromethane/methanol. 0.2 q of 2,2-dimethyl-2H-thiochromene-3-carboxylic acid is recovered (yield: 59%). ¹H NMR (DMSO): 1.56 (s, 6H, 2 CH₃), 7.16-7.20 (m, 1H, H arom), 7.25-7.2B (m, 2H, H arom), 7.33 (s, 1H, CH═), 7.43 (d, 1H, H arom), 12.75 (s, 1H, COOH).

### Step 3: Preparation of 2,2-dimethyl-2H-thiochromene-3-carboxylic acid {4-[4 (3-cyanophenyl)-piperazin-1-yl]-butyl}-amide

This compound is prepared according to the procedure for the 2H-chromene-3-carboxylic acid {4-[4-(2-methoxyphenyl)-piperazin-1-yl]-butyl}-amide of example 1, but using the acid prepared in previous step 2 and the 4-[4-(3-cyano phenyl)-piperazin-1-yl]-butylamine prepared according to the same method as in step 4 of example 1, but with corresponding reagents. 0.7 q of 2,2-dimethyl-2H-thiochromene-3-carboxylic acid {4-[4-(3-cyanophenyl)-piperazin-1-yl]-butyl}-amide is recovered (yield: 58%). ¹H NMR (DMSO): 1.50 (s large, 10H, 2 CH₂ and 2 CH₃), 2.32-2.35 (m, 2H, CH₂), 2.69 (s large, 4H, CH₂), 3.15-3.21 (m, 6H, CH₂), 6.71 (s, 1H, H arom), 7.13-7.39 (m, 8H, H arom and CH═), 8.35 (t, 1H, NHCO).

Preparation of the salt: Dissolve 0.7 g of the base (1.52 mmol, 1 eq) in 5 ml of ethyl acetate. Introduce 0.7 ml of a 5 N solution of HCl in isopropanol (2.2 eq). Evaporate and take up in ethyl ether and ethyl acetate. Filter and dry the salt formed. Yield: 62%. MP=155° C. ¹H NMR (DMSO): 1.51 (s large, 8H, CH₂ and 2 CH₃), 1.75-1.79 (m, 2H, CH₂), 3.05-3.28 (m, 8H, CH₂), 3.54 (d, 2H, CH₂), 3.96 (d, 2H, CH₂), 6.79 (s, 1H, H arom), 7.15-7.26 (m, 4H, H arom), 7.34-7.46 (m, 4H, H arom), 8.39 (t, 1H, NHCO), 10.96 (s, 1H, HCl). MS (ESI, 250° C.): MH⁺=461.2 (100%).

### EXAMPLE 45

### 2,2-Dimethyl-2H-thiochromene-3-carboxylic acid {4-[4-(3-hydroxyphenyl)-piperazin-1-yl]-butyl}-amide

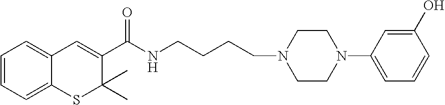

### Step 1: Preparation of 2,2-dimethyl-2H-thiochromene-3-carboxylic acid (4-hydroxybutyl)-amide

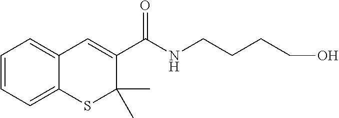

This compound is prepared according to the procedure for the 2H-thiochromene-3-carboxylic acid (4-hydroxybutyl)-amide of step 1 of example 41. 0.4 g of 2,2-dimethyl-2-H-thiochromene-3-carboxylic acid (4-hydroxy-butyl)-amide is recovered (yield: 23%). ¹H NMR (DMSO): 1.43-1.48 (m, 4H, CH₂), 1.49 (s, 6H, CH₃), 3.10-3.15 (m, 2H, CH₂), 3.39-3.43 (m, 2H, CH₂), 4.40 (t, 1H, OH), 6.69 (s, 1Hr CH═), 7.15-7.26 (m, 3H, H arom), 7.35 (d, 1H, H arom), 8.30 (t, 1H NHCO).

### Step 2: Preparation of 2,2-dimethyl-2H-thiochromene-3-carboxylic acid (4-iodobutyl)-amide

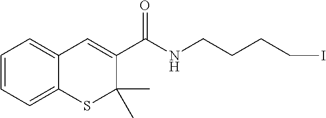

This compound is prepared according to the procedure for the 2H-thiochromene-3-carboxylic acid (4-iodobutyl)-amide of step 2 of example 41. 0.44 g of 2,2-dimethyl-2H-thiochromene-3-carboxylic acid (4-iodobutyl)-amide is recovered (yield: 80%). ¹H NMR (DMSO): 1.50 (s, 6H, CH₃), 1.52-1.59 (m, 2H, CH₂), 1.77-1.84 (m, 2H, CH₂), 3.13-3.18 (m, 2H, CH₂), 3.30-3.33 (m, 2H, CH₂), 6.71 (s, 1H, CH═), 7.15-7.26 (m, 3H, H arom), 7.35 (d, 1H, H arom), 8.36 (t, 1H, NHCO).

### Step 3: Preparation of 2,2-dimethyl-2H-thiochromene-3-carboxylic acid {4-[4-(3-hydroxyphenyl)-piperazin-1-yl]-butyl}-amide hydrochloride

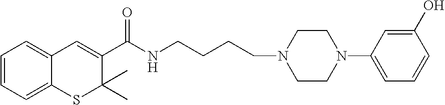

This compound is prepared according to the procedure for the 2H-thiochromene-3-carboxylic acid {4-[4-(3-hydroxyphenyl)piperazin-1-yl]-butyl}-amide of step 3 of example 41, but using the iodine derivative of previous step 2 and N-3-hydroxyphenyl piperazine. 0.4 g of 2,2-dimethyl-2H-thiochromene-3-carboxylic acid {4-[4-(3-hydroxyphenyl)-piperazin-1-yl]-butyl}-amide is recovered (yield: 77%). ¹H NMR (DMSO): 1.50 (s large, 10H, CH₂ and CH₃), 2.32-2.34 (m, 2H, CH₂N), 2.44-2.51 (m, 4H, CH₂ piperazine), 3.04-3.06 (m, 4H, CH₂ piperazine), 3.14-3.17 (m, 2H, CH₂NHCO), 6.19 (dd, 1H, H arom), 6.28 (s, 1H, H arom), 6.35 (dd, 1H, H arom), 6.71 (s, 1H, CH═), 6.96 (t, 1H, H arom), 7.16-7.26 (m, 3H, H arom), 7.35 (dd, 1H, H arom), 8.34 (t, 1H, NHCO), 9.09 (s, 1H, OH).

Preparation of the salt: Dissolve 0.4 g of the base (0.88 mmol, 1 eq) in 5 ml of methanol. Introduce 0.35 ml of a 5 N solution of HCl in isopropanol (2 eq). Evaporate and take up in acetone. Filter and dry the salt formed. Yield: 67%. MP=sticky above 90° C., truly melts at roughly 145° C. MS (ESI, 400° C.): MH⁺=452.2 (100%).

### EXAMPLE 46

### Preparation of 2H-chromene-3-carboxylic acid {4-[4-(3-hydroxymethylphenyl)-piperazin-1-yl]-butyl}-amide

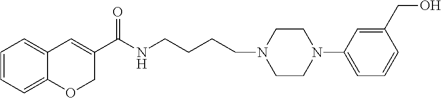

### Step 1: 2H-Chromene-3-carboxylic acid (4-hydroxy-butyl)-amide

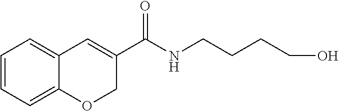

This compound is prepared according to the procedure for the 2H-thiochromene-3-carboxylic acid (4-hydroxy-butyl)-amide in step 1 of example 41, but using the 2H-chromene-3-carboxylic acid prepared in step 2 of example 1. 1.5 g of 2H-chromene-3-carboxylic acid {4 hydroxy-butyl}-amide is recovered (yield: 56%). ¹H NMR (DMSO-d₆): 1.40-1.54 (m, 4H, CH₂), 3.14-3.19 (m, 2H, CH₂N), 3.38-3.43 (m, 2H, CH₂OH), 4.40 (t, 1H, OH), 4.89 (d, 2H, J_{HH}=11.2 Hz, CH₂O), 6.83 (d, 1H, H arom), 6.93-6.97 (m, 1H, H arom), 7.19-7.24 (m, 3H, 2H arom and CH═), 8.20 (t, 1H, NHCO).

### Step 2: 2H-Chromene-3-carboxylic acid (4-iodo-butyl)-amide

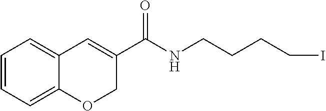

This compound is prepared according to the procedure for the 2H-thiochromene-3-carboxylic acid (4-iodo-butyl)-amide in step 2 of example 41. 0.7 g of 2H-chromene-3-carboxylic acid (4-iodo-butyl)-amide is obtained in the form of an orange solid (yield: 50%). ¹H NMR (DMSO-d₆): 1.52-1.59 (m, 2H, CH₂), 1.75-1.82 (m, 2H, CH₂), 3.16-3.21 (m, 2H, CH₂), 3.29-3.32 (m, 2H, CH₂), 4.89 (s, 2H, CH₂O), 6.84 (d, 1H, H arom), 6.95 (t, 1H, H arom), 7.20-7.24 (m, 3H, 2H arom and CH═), 8.24 (t, 1H, NHCO).

### Step 3: Preparation of (3 piperazin-1-yl-phenyl)-methanol

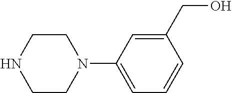

This piperazine is prepared from 4 (3 hydroxymethyl-phenyl)-piperazine-1-carboxylic acid tert-butyl ester, itself prepared by reduction in NaBH₄ of the corresponding aldehyde, analogous to Bioorg. Med. Chem. Lett. 2003, 13, 3793. In a 250 ml round bottomed flask, introduce 1 g of 4-(3-hydroxymethyl-phenyl)-piperazine-1-carboxylic acid tert-butyl ester (4 mmol, 1 eq) and then add 25 ml of ethanol and 25 ml of 30% HCl. Leave for 8 h under agitation at room temperature. The ethanol is then concentrated and the mixture made more basic. Extract with dichloromethane. The organic phase is dried on Na₂SO₄, filtered, evaporated and purified on silica using a dichloromethane/ethyl acetate gradient. 0.6 g of (3-piperazin-1-yl-phenyl)-methanol is recovered (yield: 79%). ¹H NMR (DMSO): 2.80-2.83 (m, 4H, CH₂ piperazine), 3.00-3.02 (m, 4H, CH₂ piperazine), 4.42 (d, 2H, CH₂OH), 5.06 (t, 1H, OH), 6.71 (d, 1H, H arom), 6.76 (dd, 1H, H arom), 6.86 (s, 1H, H arom), 7.13 (t, 1H, H arom).

### Step 4: 2H-Chromene-3-carboxylic acid {4-[4-(3-hydroxymethylphenyl)-piperazin-1-yl]-butyl}-amide

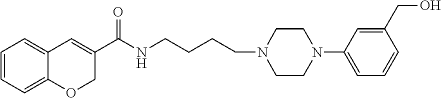

This compound is prepared according to the procedure for the 2H-thiochromene-3-carboxylic acid {4-[4-(3-hydroxyphenyl)-piperazin-1-yl]-butyl}-amide of step 3 of example 41, but using the reagents prepared in previous step 2 (iodine derivative) and in previous step 3. 0.2 g of 2H-chromene-3-carboxylic acid {4-[4-(3-hydroxymethylphenyl)-piperazin-1-yl]-butyl}-amide is recovered (yield: 24%). ¹H NMR (DMSO): 1.48-1.51 (m, 4H, CH₂), 2.32-2.34 (m, 2H, CH₂N), vers 2.50 cache par le pic du DMSO (4H, CH₂ piperazine), 3.09-3.12 (m, 4H, CH₂ piperazine), 3.17-3.20 (m, 2H, CH₂NHCO), 4.42 (d, 2H, CH₂OH), 4.89 (d, 2H, J_{HH}=1.2 Hz, CH₂O), 5.06 (t, 1H, OH), 6.72 (d, 1H, H arom), 6.78 (dd, 1H, H arom), 6.84 (d, 1H, H arom), 6.87 (s, 1, H arom), 6.94 (td, 1H, H arom), 7.14 (t, 1H, H arom), 7.19-7.23 (m, 3H, H arom and CH═), 8.21 (t, 1H, NHCO).

Preparation of the salt: Dissolve 0.2 q of the base (0.5 mmol, 1 eq) in 5 ml of dichloromethane. Introduce 0.2 ml of a 5 N solution of HCl in isopropanol (2.2 eq). Evaporate and take up in ethyl ether. Filter and dry the salt formed. Yield: 55%. MP=194° C. ¹H NMR (DMSO): 1.51-1.57 (m, 2H, CH₂), 1.68-1.74 (m, 2H, CH₂), 2.98-3.25 (m, 5H, CH₂), 3.50-3.52 (m, 2H, CH₂), 3.79-3.82 (m, 2H, CH₂), 4.45 (s, 2H, CH2OH), 4.91 (d, 2H, J_{HH}=1.2 Hz, CH₂O), 6.82-6.88 (m, 3H, H arom), 6.95-6.97 (m, 2H, H arom), 7.19-7.26 (m, 4H, H arom and CH═), 8.31 (t, 1H, NHCO), 9.85 (s, 1H, HCl). MS (APCI+, 400° C.): MH⁺=422.2 (100%).

Similarly, but using the 2H-thiochromene-3-carboxylic acid {4-iodo-butyl}-amide obtained in step 2 of example 41, the compounds of the following example are obtained:

### EXAMPLE 47

### Preparation of 2H-thiochromene-3-carboxylic acid {4-[4-(3-hydroxymethylphenyl)-piperazin-1-yl]-butyl}-amide

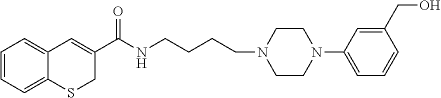

Analysis: C₂₅H₃₁N₃O₂S, MW=437.61

### EXAMPLE 48

### 2,2-Dimethyl-2H-thiochromene-3-carboxylic acid {4-[4-(2-cyanophenyl)-piperazin-1-yl]-butyl}-amide

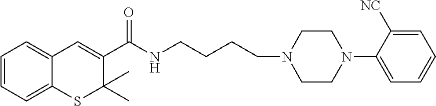

This compound is prepared according to the procedure for the 2H-chromene-3-carboxylic acid {4-[4-(2-methoxyphenyl)-piperazin-1-yl]-butyl}-amide of example 1, but using the acid prepared in step 2 of example 44 and the 4-[4-(3-cyano-phenyl)piperazin-1-yl]-butylamine prepared according to the same method as in step 4 of example 1, but with corresponding reagents. 2,2-dimethyl-2H-thiochromene-3-carboxylic acid {4-[4-(2-cyanophenyl)-piperazin-1-yl]-butyl}-amide is obtained. Analysis: C₂₇H₃₂N₄OS, MW: 460.65.

### EXAMPLE 49

### 5-Bromo-8-methoxy-2H-chromene-3-carboxylic acid {4-[4 (2-fluorophenyl)-piperazin-1-yl]-butyl}-amide hydrochloride

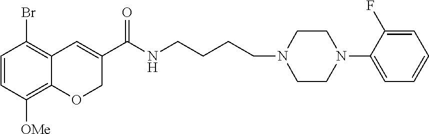

### Step 1: Preparation of 5-bromo-8-methoxy-2H-chromene-3-carbonitrile

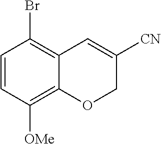

According to the same procedure as in step 1 of example 1, 5-bromo-8-methoxy-2H-chromene-3-carbonitrile is prepared from 6-bromo-3-methoxy salicylaldehyde. Yield=40%. ¹H NMR (DMSO): 3.78 (s, 3H, —OCH₃), 4.87 (s, 2H, O—CH₂—), 7.05 (d, 1H, H arom), 7.25 (d, 1H, H arom), 7.54 (s, 1H, H4). C₁₁H₉BrO₄. MW: 285.10.

### Step 2: Preparation of 5-bromo-8-methoxy-2H-chromene-3-carboxylic acid

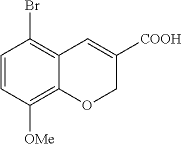

5-Bromo-8-methoxy-2H-chromene-3-carboxylic acid is prepared according to the same procedure as in step 2 of example 1. Yield=96%. ¹H NMR (DMSO): 3.77 (s, 3H, —OCH₃), 4.89 (s, 2H, O—CH₂—), 6.99 (d_{,} 1H, H arom), 7.19 (d, 1H, H arom), 7.42 (s, 1H, H₄), 13.5 (s, ech. —COOH). F: 121° C. (decom.)

### Step 3: Preparation of 5-bromo-8-methoxy-2H-chromene-3-carboxylic acid {4-[4-(2-fluororophenyl)piperazin-1-yl]-butyl}-amide

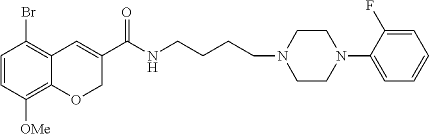

This compound is prepared according to the procedure of example 1, but using corresponding reagents. The acid used is the 5-bromo-8-methoxy-2H-chromene-3-carboxylic acid obtained in previous step 2, and the amine used is prepared from 1-(2-fluorophenyl)piperazine according to the same procedure as for obtaining the 4-[4-(2 methoxy-phenyl)-piperazin-1-yl]-butylamine of steps 3 and 4 of example 1. 5-Bromo-8-methoxy-2H-chromene-3-carboxylic acid {4-[4-(2-fluororophenyl)-piperazin-1-yl]-butyl}-amide is obtained. ¹H NMR (CDCl₃ base): 1.65-1.71 (m, 4H, —CH₂—CH₂—), 2.45-2.48 (t, 2H, —CH₂—N), 2.63-2.65 (m, 4H, H-piperazine), 3.08-3.10 (m, 4H, H-piperazine), 3.38-3.51 (m, 2H, —CH₂—N—CO—), 3.86 (s, 3H —OCH₃), 5.02 (s, 2H, O—CH₂), 6.54 (s, 1H, —NH), 6.69-7.26 (m, 7H, H arom+H₄). Analysis (salt): C₂₅H₂₉O₃N₃BrF-2HCl. Mass=605.79. MS (ESI⁺, 400° C.): MH⁺=520 (100%). MP=157° C.

### EXAMPLE 50

### 5-Bromo-8-methoxy-2H-chromene-3-carboxylic acid {4-[4-(2-methoxyphenyl)piperazin-1-yl]-butyl}-amide

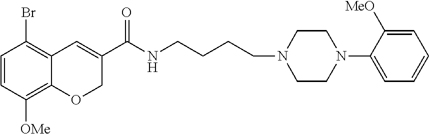

This derivative is obtained according to the procedure of previous example 49, and by analogy with that of example 1, but with corresponding reagents. ¹H NMR (CDCl₃ base): 1.64-1.71 (m, 4H, —CH₂—CH₂—), 2.47 (t, 2H, —CH₂—N), 2.61-2.66 (m, 4H, H-piperazine), 3.07 (m, 4H, H piperazine), 3.38-3.43 (m, 2H, —CH₂—N—CO—), 3.85-3.86 (2 s, 6H, —OCH₃ & —OCH₃), 5.02 (s, 2H, O—CH₂), 6.73 (s, 1H, —NH), 6.80-7.26 (m, 7H, H arom+H₄). Analysis (salt): C₂₆H₃₂O₄N₃Br—HCl Mass=566.93. MS (APCI⁺, 600° C.): MH⁺=532.2 (100%). MP=176° C.

### EXAMPLE 51

### 5-Bromo 8 methoxy-2H-chromene-3-carboxylic acid {4-[4-(2,3-dichlorophenyl)-piperazin-1-yl]-butyl}-amide

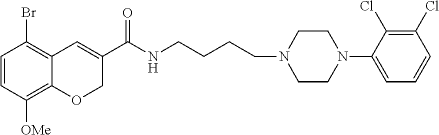

This compound is prepared in a manner analogous to previous example 49, and by analogy with that of example 1, but with corresponding reagents. ¹H NMR (CDCl₃ base): 1.63-1.73 (m, 4H, —CH₂—CH₂), 2.48 (t, 2H, —CH₂—N), 2.61-2.65 (m, 4H, H-piperazine), 3.04 (m, 4H, H-piperazine), 3.38-3.43 (m, 2H, —CH₂—N—CO—), 3.86 (s, 3H —OCH₃), 5.02 (s, 2H, O—CH₂), 6.67 (s, 1H, —NH), 6.82-7.15 (m, 6H, H arom+H₄). Analysis (salt): C₂₅H₂₈O₃N₃BrCl₂—HCl. Mass=605.79. MS (ESI⁺, 400° C.): MH⁺=570 (100%). MP=205° C.

### EXAMPLE 52

### 5-Bromo-8-methoxy-2H-chromene-3-carboxylic acid {4-[4-(3-cyanophenyl)piperazin-1-yl]-butyl}-amide

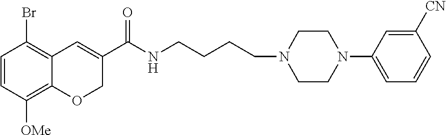

This compound is prepared in a manner analogous to previous example 49, and by analogy with that of example 1, but with corresponding reagents. ¹H NMR (DMSO base): 1.64-1.66 (m, 4H, —CH₂—CH₂—), 2.33-2.34 (t, 2H, —CH₂—N), 2.50-2.51 (m, 4H, H-piperazine), 3.20-3.21 (m, 4H, H-piperazine), 3.32 (m, 2H, —CH₂—N—CO—), 3.77 (s, 3H —OCH₃), 4.87 (s, 2H, O—CH₂), 6.31 (s, 1H, —NH), 6.94-7.35 (m, 7H, H arom+H₄), 8.43 (s, 1H, NH). Analysis (salt): C₂₆H₂₉O₃N₄Br—HCl Mass=561.91. MS (ESI⁺, 400° C.): MH⁺=525.1 (100%). MP=155° C.

### EXAMPLE 53

### 2H Chromene-3-carboxylic acid {4-[4-(3-hydroxyphenyl)-piperazin-1-yl]-butyl}-amide

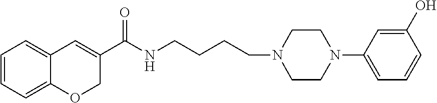

This compound is prepared according to the procedure for the 2H-thiochromene-3-carboxylic acid {4-[4-(3-hydroxyphenyl)-piperazin-1-yl]-butyl}-amide of step 4 in example 46, but using the corresponding reagent, i.e., the iodine derivative prepared in step 2 of example 46 and N-(3-hydroxyphenyl)piperazine. 0.25 g of 2H-chromene-3-carboxylic acid {4-[4-(3-hydroxyphenyl)-piperazin-1-yl]-butyl}-amide is recovered (yield=25%). ¹H NMR (DMSO): 1.48-1.50 (m, 4H, CH₂), 2.30-2.33 (m, 2H, CH₂N), 2.45-2.48 (m 4H, CH₂ piperazine), 3.04-3.06 (m, 4H, CH₂ piperazine), 3.16-3.19 (m, 2H, CH₂NHCO), 4.89 (d, 2H, J_{HH}=0.8 Hz, CH₂O), 6.19 (d, 1H, H arom), 6.28 (s, 1H, H arom), 6.35 (d, 1H, H arom), 6.83 (d, 1H H arom), 6.93-6.98 (m, 2H, H arom), 7.20-7.23 (m, 3H, H arom and CH═), 8.21 (t, 1H, NHCO), 9.08 (s, 1H, OH).

Preparation of the salt: Dissolve 0.25 g of the base (0.61 mol, 1 eq) in 5 ml of ethyl acetate. Introduce 0.3 ml of a 5 N solution of HCl in isopropanol (2.2 eq). Evaporate and take up in pentane. Filter and dry the salt formed. Yield: 68%. MP=199° C. ¹H NMR (DMSO): 1.49-1.56 (m, 2H, CH₂), 1.70-1.76 (m, 2H, CH₂), 3.00-3.16 (m, 6H, CH₂), 3.19-3.24 (m, 2H, CH₂), 3.51-3.54 (m, 2H, CH₂), 3.71-3.74 (m, 2H, CH₂), 4.91 (d, 2H, JHH=0.8 Hz, CH₂O), 6.30 (dd, 1H, H arom), 6.36 (s large, 1H, H arom), 6.43 (dd, 1H, H arom), 6.84 (d, 1H, H arom), 6.95 (td, 1H, H arom), 7.03 (t, 1H, H arom), 7.21-7.25 (m, 2H, H arom), 7.27 (s, 1H, CH═), 8.33 (t, 1H, NHCO), 9.30 (s large, 1H, OH), 10.25 (s, 1H, HCl). MS (ESI, 250° C.): MH⁺=408.2 (100%).

Similarly, but with corresponding reagents, the following compounds are prepared:

### EXAMPLE 54

### 6-Methoxy-2H-chromene-3-carboxylic acid {4-[4-(2-methoxyphenyl)piperazin-1-yl]-butyl}-amide

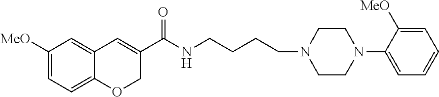

This derivative is obtained according to the procedure of example 1, with the 6-methoxy-2H-chromene-3-carboxylic acid obtained according to J. Med. Chem. 1988, 31, 688. ¹H NMR (CDCl₃ base): 1.64-1.66 (m, 4H, —CH₂—CH₂—), 2.46 (t, 2H, —CH₂—N), 2.66 (m, 4H, H-piperazine), 3.09 (m, 4H, H piperazine), 3.37-3.40 (t, 2H, —CH₂—N—CO—), 3.72 (s, 3H, OCH₃), 3.86 (s, 3H, —OCH₃), 4.94 (s, 2H, O—CH₂), 6.54 (s, 1H, —NH), 6.62-7.01 (m, 7H, H arom+H₄). Analysis (salt): C₂₆H₃₃O₄N₃—HCl. Mass=488.03. MS (ESI⁺, 400° C.): MH⁺=452.3 (100%). MP=165° C.

### EXAMPLE 55

### 6-Methoxy-2H-chromene-3-carboxylic acid {4-[4-(2,3-dichlorophenyl)-piperazin-1-yl]-butyl}-amide

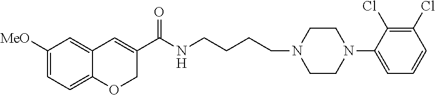

This derivative is obtained according to the procedure of example 1, but with corresponding reagents. ¹H NMR (CDCl₃ base): 1.67-1.73 (m, 4H, —CH₂—CH₂—), 2.48 (t, 2H, —CH₂—N), 2.65 (m, 4H, H-piperazine), 3.04 (m, 4H, H piperazine), 3.38-3.43 (m, 2H, —CH2-N—CO—), 3.86 (s, 3H, OCH₃), 5.03 (s, 2H, O—CH₂), 6.67 (s, 1H, —NH), 6.82-7.14 (m, 5H, H arom+H₄). Analysis (salt): C₂₅H₂₈O₃Cl₂N₃Br—HCl. Mass=605.79. MS (ESI⁺, 400° C.): MH⁺=570.0 (100%). MP=205° C.

### EXAMPLE 56

### 6-Methoxy-2H-chromene-3-carboxylic acid {4-[4-(2-fluorophenyl)piperazin-1-yl]-butyl}-amide

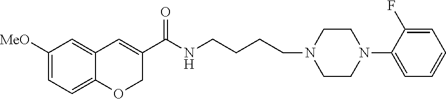

This derivative is obtained according to the procedure of example 1, but with corresponding reagents. ¹H NMR (CDCl₃ base): 1.64-1.66 (m, 4H, —CH₂—CH₂—), 2.46 (t, 2H, —CH₂—N), 2.66 (m, 4H, H-piperazine), 3.09 (m, 4H, H-piperazine), 3.37-3.40 (t, 2H, —CH₂—N—CO—), 3.72 (s, 3H, OCH₃), 3.86 (s, 3H, —OCH₃), 4.94 (s, 2H, O—CH₂), 6.54 (s, 1H, —NH), 6.62-7.01 (m, 7H, H arom+H14). Analysis (salt): C₂₆H₃₃O₄N₃—HCl. Mass=488.03. MS (ESI⁺, 400° C.): MH⁺=452.3 (100%). MP=154° C.

### EXAMPLE 57

### 6-Methoxy-2H-chromene-3-carboxylic acid {4-[4-(3-cyanophenyl)-piperazin-1-yl]-butyl}-amide

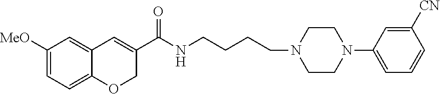

This derivative is obtained according to the procedure of example 1, but with corresponding reagents. ¹H NMR (CDCl₃ base): 1.64-1.66 (m, 4H, —CH₂—CH₂—), 2.44 (t, 2H, —CH₂—N), 2.60 (m, 4H, H-piperazine), 3.21 (m, 4H, H-piperazine), 3.37-3.42 (m, 2H, —CH₂—N—CO—), 3.75 (s, 3H, OCH₃), 4.93 (s, 2H, O—CH₂), 6.31 (s, 1H, —NH), 6.75-7.40 (m, 7H, H arom+H₄). Analysis (salt): C₂₆H₃₀O₃N₄—HCl Mass=483.01. MS (ESI⁺, 400° C.): MH⁺=447.3 (100%). MP=159° C.

In an analogous manner, the following compounds are obtained but with corresponding reagents:

### EXAMPLE 58

### 2H-Chromene-3-carboxylic acid {4-[4-(2-hydroxyphenyl)-piperazin-1-yl]-butyl}-amide

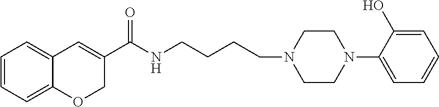

¹H NMR (CDCl₃ base): 1.59-1.65 (m, 4H, —CH₂—CH₂—), 2.45 (t, 2H, —CH₂—N), 2.62 (m, 4H, H-piperazine), 2.89 (m, 4H, H-piperazine), 3.40 (m, 2H, —CH₂—N—CO—), 5.01 (s, 2H, O—CH₂), 6.31 (s, 1H, —NH), 6.81-7.26 (m, 9H, H arom+H₄). Analysis (salt): C₂₄H₂₉O₃N₃—HCl. Mass=443.98. MS (ESI⁺, 250° C.): MH⁺=408.1 (100%). MP=189° C.

### EXAMPLE 59

### 2H-Chromene-3-carboxylic acid {4-[4-(4-hydroxyphenyl)-piperazin-1-yl]-butyl}-amide

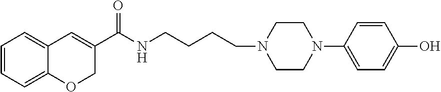

¹H NMR (CDCl₃ base): 1.65-1.67 (m, 4H, —CH₂—CH₂—), 2.51 (t, 2H, —CH₂—N), 2.71 (m, 4H, H-piperazine), 3.11 (m, 4H, H-piperazine), 3.41 (m, 2H, —CH₂—N—CO—), 5.00 (s, 2H, OCH₂), 6.51 (s, 1H, —NH), 6.74-7.21 (m, 9H, H arom+H4). Analysis (salt): C₂₄H₂₉O₃N₃—HCl. Mass=443.98. MS (ESI⁺, 250° C.): MH⁺=408.3 (100%). MP=236° C.

### EXAMPLE 60

### 2H-Chromene-3-carboxylic acid {4-[4-(4-cyanophenyl)-piperazin-1-yl]-butyl}-amide

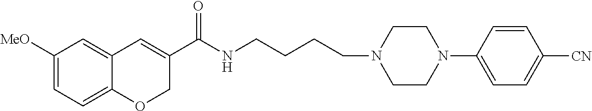

Analysis: C₂₆H₃₀N₄O₃. MW=446.55

### EXAMPLE 61

### 2H-Chromene-3-carboxylic acid {4-[4-(2-cyanophenyl)-piperazin-1-yl]-butyl}-amide

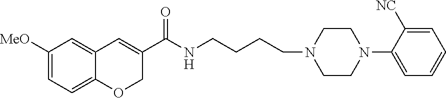

Analysis: C₂₆H₃₀N₄O₃. MW=446.55

### EXAMPLE 62

### 2H-Chromene-3-carboxylic acid {4-[4-(3-methoxyphenyl)-piperazin-1-yl]-butyl}-amide

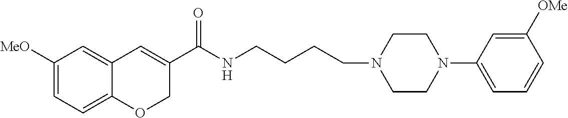

Analysis: C₂₆H₃₃N₃O₄. MW=451.57

### EXAMPLE 63

### 2H-Chromene-3-carboxylic acid {4-[4-(3,4-dimethoxyphenyl)piperazin 1-yl]-butyl}-amide

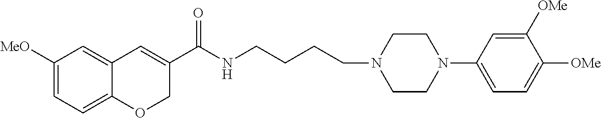

Analysis: C₂₇H₃₅N₃O₅. MW=481.60

### EXAMPLE 64

### 2H-Chromene-3-carboxylic acid {4-[4-(3,4-methylenedioxyphenyl)-piperazin-1-yl]-butyl}-amide

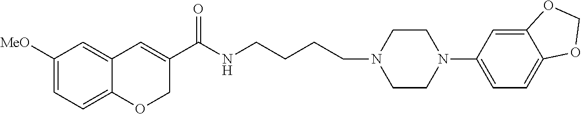

Analysis: C₂₆H₃₁N₃O₅. MW=465.55

Other examples of compounds in conformity with the present invention are listed in following table I.

| Example | | |
|---|---|---|
| no. | Name | |
| 65 | 7-methoxy-2H- chromene-3-carboxylic acid {4-[4-(2-methoxy- phenyl)-piperazin-1-yl]- butyl}-amide | 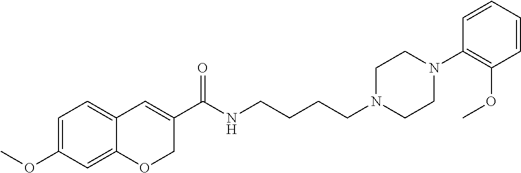 |
| 66 | 7-methoxy-2H- chromene-3-carboxylic acid {4-[4-(3-cyano- phenyl)-piperazin-1-yl]- butyl}-amide | 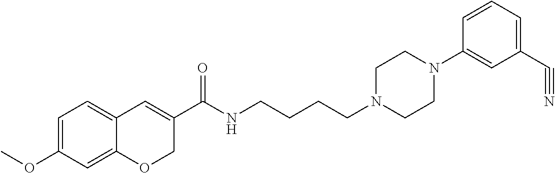 |
| 67 | 7-methoxy-2H- chromene-3-carboxylic acid {4-[4-(2,3-dichloro- phenyl)-piperazin-1-yl]- butyl}-amide | 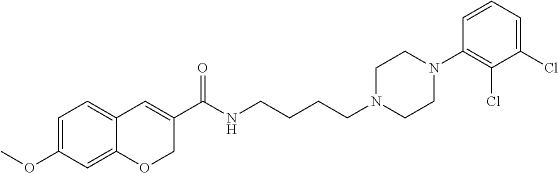 |
| 68 | 7-methoxy-2H- chromene-3-carboxylic acid {4-[4-(3-hydroxy- phenyl)-piperazin-1-yl]- butyl}-amide | 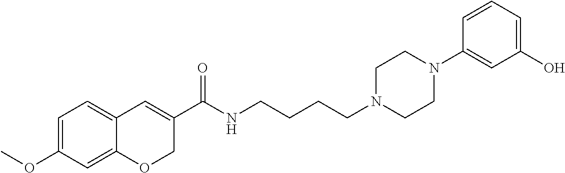 |
| 69 | 7-methoxy-2H- chromene-3-carboxylic acid {4-[4-(2,3-dihydro- benzo[1,4]dioxin-6-yl)- piperazin-1-yl]butyl}- amide | 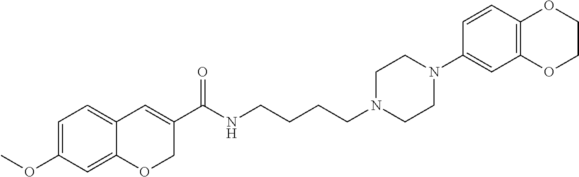 |
| 70 | 7-methoxy-2H- chromene-3-carboxylic acid {4-[4-(3-methyloxy- carbonyl)-piperazin-1- yl]-butyl}-amide | 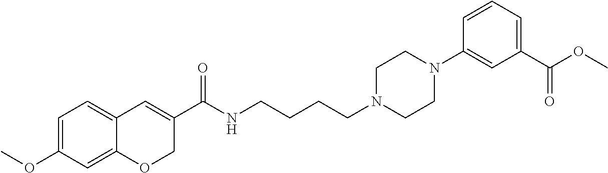 |
| 71 | 6-methoxy-2H- chromene-3-carboxylic acid {4-[4-(2,4-dichloro- phenyl)-piperazin-1-yl]- butyl}-amide | 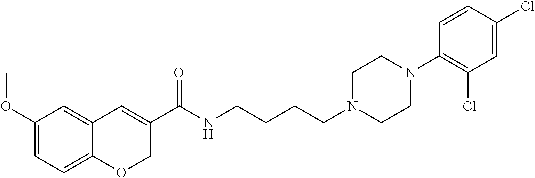 |
| 72 | 3(4-{4-[(6-methoxy-2H- chromene-3-carbonyl)- amino]-butyl}-piperazin- 1-yl)-benzoic acid ethyl ester | 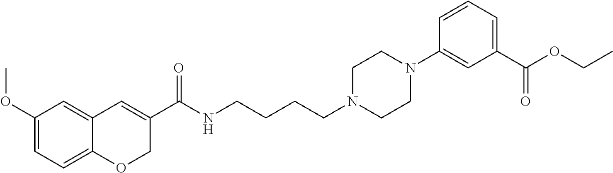 |
| 73 | 6-methoxy-2H- chromene-3-carboxylic acid {4-[4-(3-amino- phenyl)-piperazin-1-yl]- butyl}-amide | 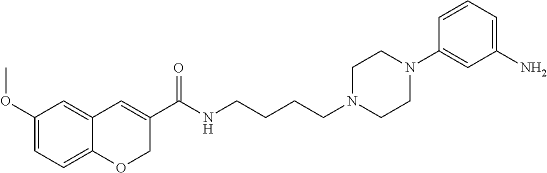 |
| 74 | 6-methoxy-2H- chromene-3-carboxylic acid {4-[4-(3-nitro- phenyl)-piperazin-1-yl]- butyl}-amide | 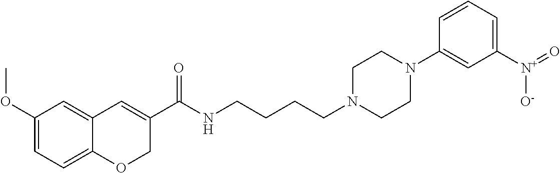 |
| 75 | 6-methoxy-2H- chromene-3-carboxylic acid {4-[4-(3- hydroxymethyl-phenyl)- piperazin-1-yl]-butyl}- -amide | 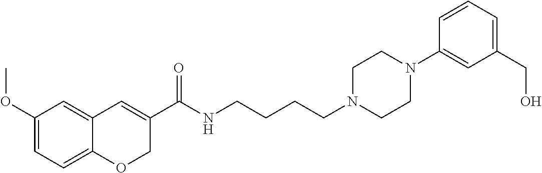 |
| 76 | 6-methoxy-2H- chromene-3-carboxylic acid {4-[4-(3- acetylamino-phenyl)- piperazin-1-yl]-butyl}- -amide | 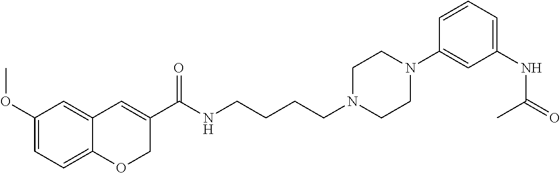 |
| 77 | 6-methoxy-2H- chromene-3-carboxylic acid {4-[4-(3-hydroxy- phenyl)-piperazin-1-yl]- butyl}-amide | 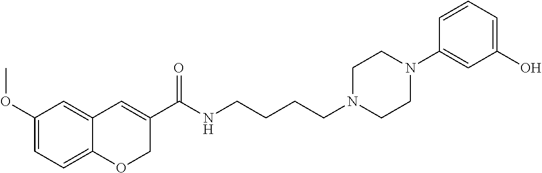 |
| 78 | 6-methoxy-2H- chromene-3-carboxylic acid {4-[4-(2,3-benzo- 1,4-dioxanyl-)-piperazin- 1-yl]-butyl}-amide | 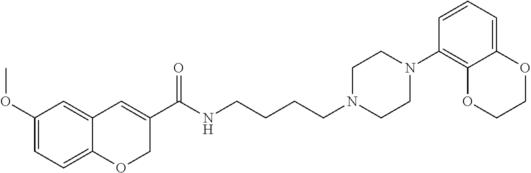 |
| 79 | 6-methoxy-2H- chromene-3-carboxylic acid {4-[4-(3,4-benzo- 1,4-dioxanyl-)-piperazin- 1-yl]-butyl}-amide | 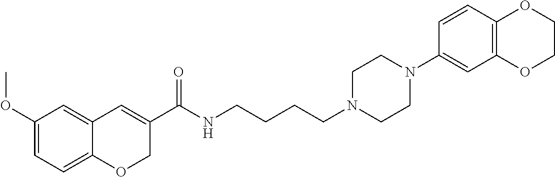 |
| 80 | 6-methoxy-2H- chromene-3-carboxylic acid {4-[4-(3- methylamino-carbonyl)- piperazin-1-yl]-butyl}- amide | 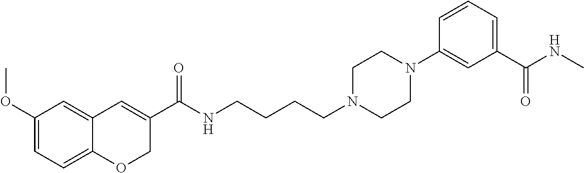 |
| 81 | 6-methoxy-2H- chromene-3-carboxylic acid {4-[4-(3-mesylamino- phenyl)-piperazin-1-yl]- butyl}-amide | 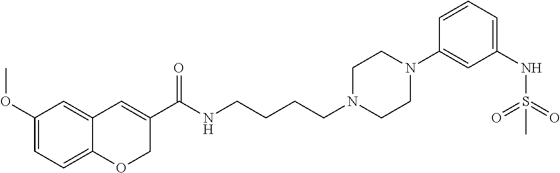 |
| 82 | 6-methoxy-2H- chromene-3-carboxylic acid {4-[4-(3-methyloxy- carbonyl-)-piperazin-1- yl]-butyl}-amide | 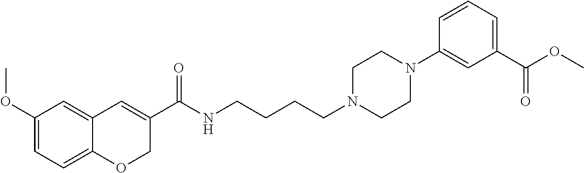 |
| 83 | 6-chloro-2H-chromene- 3-carboxylic acid {4-[4- (2,4-dichloro-phenyl)- piperazin-1-yl]-butyl}- amide | 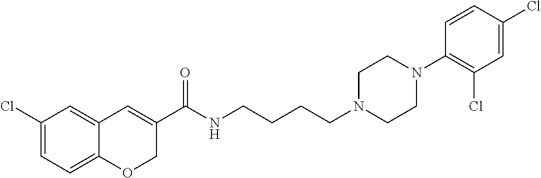 |
| 84 | 6-chloro-2H-chromene- 3-carboxylic acid {4-[4-(3- nitro-phenyl)-piperazin-1- yl]-butyl}-amide | 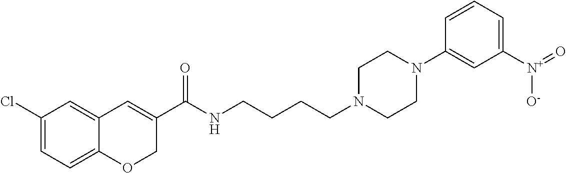 |
| 85 | 6-chloro-2H-chromene- 3-carboxylic acid {4-[4-(3- amino-phenyl)-piperazin- 1-yl]-butyl}-amide | 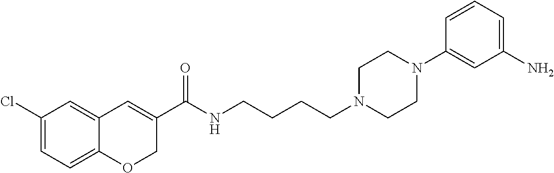 |
| 86 | 6-chloro-2H-chromene- 3-carboxylic acid {4-[4-(3- acetylamino-phenyl)- piperazin-1-yl]-butyl}- amide | 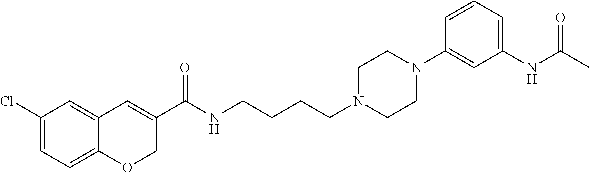 |
| 87 | 6-chloro-2H-chromene- 3-carboxylic acid {4-[4-(3- hydroxy-phenyl)- piperazin-1-yl]-butyl}- amide | 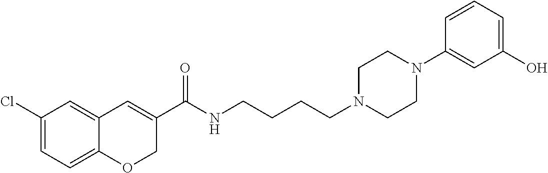 |
| 88 | 6-chloro-2H-chromene- 3-carboxylic acid {4-[4-(3- hydroxymethyl-phenyl)- piperazin-1-yl]-butyl}- amide | 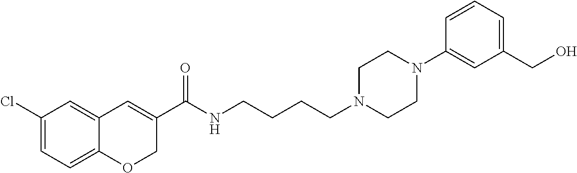 |
| 89 | 6-chloro-2H-chromene- 3-carboxylic acid {4-[4-(3- mesylamino-phenyl}- piperazin-1-yl]-butyl}- amide | 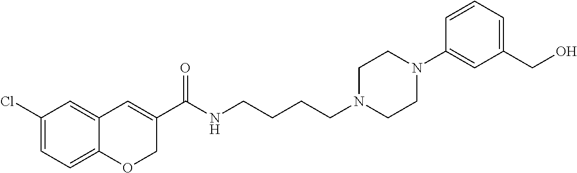 |
| 90 | 6-fluoro-2H-chromene-3- carboxylic acid {4-[4-(2,3- dichloro-phenyl)- piperazin-1-yl]-butyl}- amide | 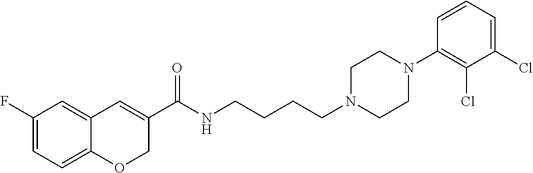 |
| 91 | 6-fluoro-2H-chromene-3- carboxylic acid {4-[4-(2- methoxy-phenyl)- piperazin-1-yl]-butyl}- amide | 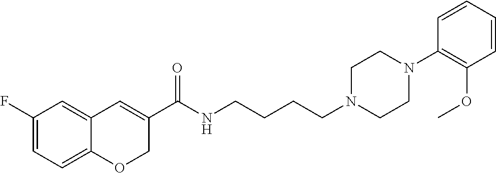 |
| 92 | 6-fluoro-2H-chromene-3- carboxylic acid {4-[4-(3- cyano-phenyl)-piperazin- 1-yl]-butyl}-amide | 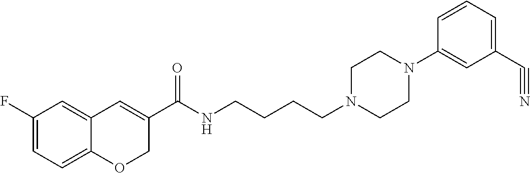 |
| 93 | 6-fluoro-2H-chromene-3- carboxylic acid {4-[4-(3- acetylamino-phenyl)- piperazin-1-yl]-butyl}- amide | 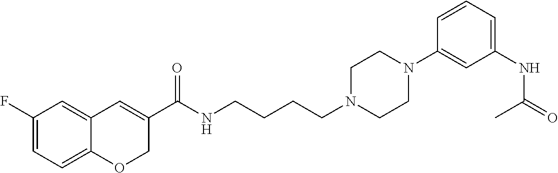 |
| 94 | 6-fluoro-2H-chromene-3- carboxylic acid {4-[4-(3- hydroxy-phenyl)- piperazin-1-yl]-butyl}- amide | 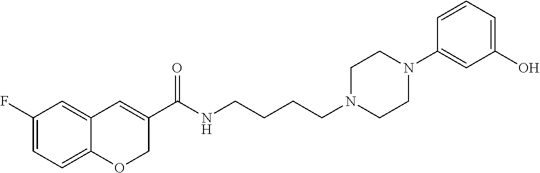 |
| 95 | 6-fluoro-2H-chromene-3- carboxylic acid {4-[4-(3- nitro-phenyl)-piperazin-1- yl]-butyl}-amide | 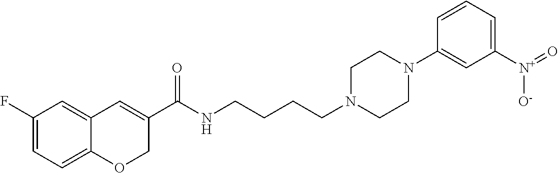 |
| 96 | 6-fluoro-2H-chromene-3- carboxylic acid {4-[4-(3- mesylamino-phenyl)- piperazin-1-yl]-butyl}- amide | 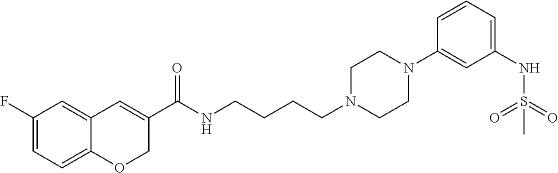 |
| 97 | 6-fluoro-2H-chromene-3- carboxylic acid {4-[4-(3- amino-phenyl)-piperazin- 1-yl]-butyl}-amide | 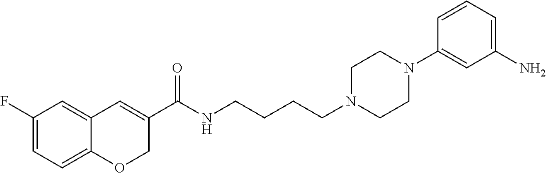 |
| 98 | 6-fluoro-2H-chromene-3- carboxylic acid {4-[4-(2,3- benzo-1,4-dioxanyl)- piperazin-1-yl]-butyl}- amide | 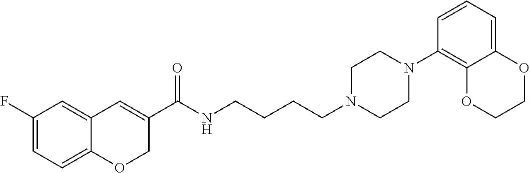 |
| 99 | 6-fluoro-2H-chromene-3- carboxylic acid {4-[4-(3- methyloxy-carbonyl)- piperazin-1-yl]-butyl}- amide | 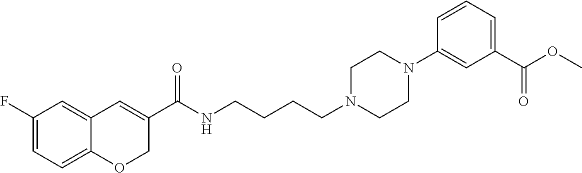 |
| 100 | 6-fluoro-5-(4-{4-[2H- chromene-3-carbonyl)- amino]-butyl}-piperazin- 1-yl)-benzofuran-2- carboxylic acid methyl ester | 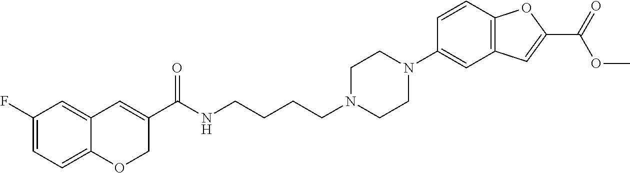 |
| 101 | 2H-chromene-3- carboxylic acid {4-[4- (3,4,5-trimethoxy-phenyl)- piperazin-1-yl]-butyl}- amide | 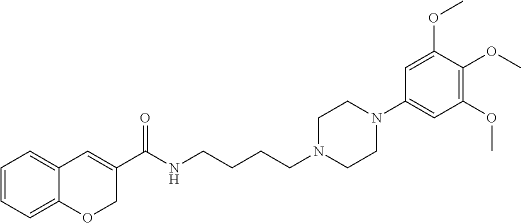 |
| 102 | 2H-chromene-3- carboxylic acid {4-[4-(1H- indol-4-yl)-piperazin-1- yl]-butyl}-amide | 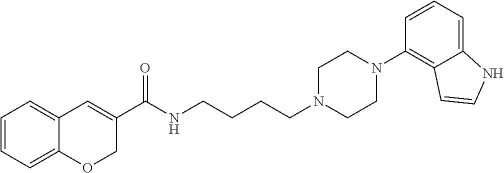 |
| 103 | 2H-chromene-3- carboxylic acid {4-[4-(3- amino)-piperazin-1-yl]- butyl}-amide | 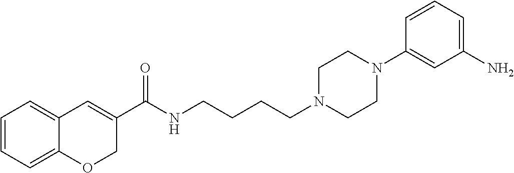 |
| 104 | 2H-chromene-3- carboxylic acid {4-[4-(2,3- dihydro-benzo[1,4]dioxin- 6-yl)-piperazin-1-yl]- butyl}-amide | 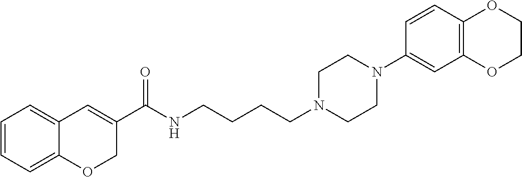 |
| 105 | 2H-chromene-3- carboxylic acid {4-[4-(2,3- dihydro-benzo[1,4]dioxin- 5-yl)-piperazin-1-yl]- butyl}-amide | 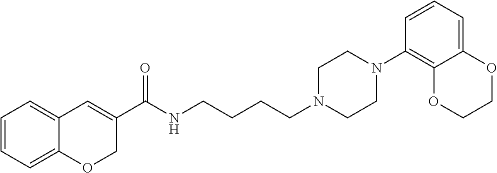 |
| 106 | 5-(4-{4-[2H-chromene-3- carbonyl)-amino]-butyl]- piperazin-1-yl)- benzofuran-2-carboxylic acid methyl ester | 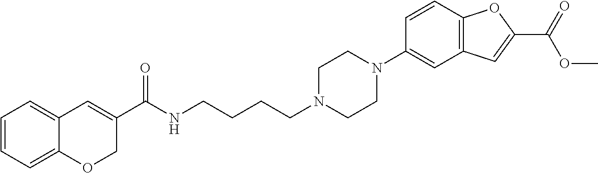 |
| 107 | 2H-chromene-3 carboxylic acid {4-[4-(2,3- dihydro-1H-indol-4-yl)- piperazin-1-yl]-butyl}- amide | 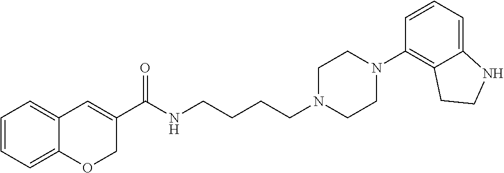 |
| 108 | 2H-chromene-3- carboxylic acid {4-[4-(3- mesylamino-phenyl)- piperazin-1-yl]-butyl}- amide | 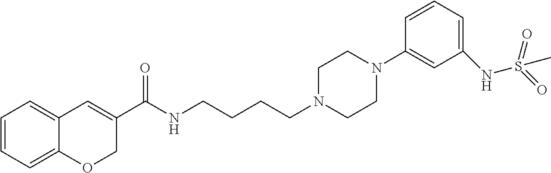 |
| 109 | 2H-chromene-3- carboxylic acid {4-[4-(1- acetyl-2,3-dihydro-1H- indol-4-yl)-piperazin-1- yl]-butyl}-amide | 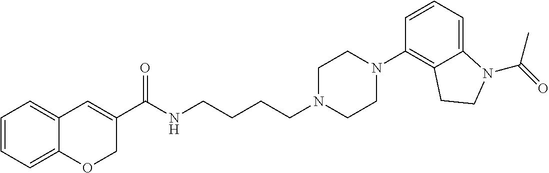 |
| 110 | 2H-chromene-3- carboxylic acid {4-[4-(2- oxo-2,3-dihydro- benzoxazol-7-yl)- piperazin-1-yl]-butyl}- amide | 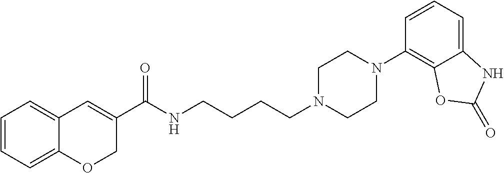 |
| 111 | 2H-chromene-3- carboxylic acid {4-[4-(2,3- dihydro-benzofuran-7-yl)- piperazin-1-yl]-butyl} amide | 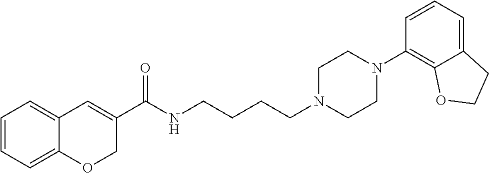 |
| 112 | 2H-thiochromene-3- carboxylic acid {4-[4-(2,3- dimethyl)-piperazin-1-yl]- butyl}-amide | 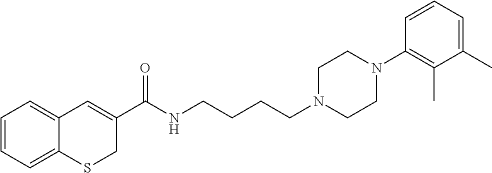 |
| 113 | 2H-thiochromene-3- carboxylic acid {4-[4-(3- methyl-phenyl)-piperazin- 1-yl]-butyl}-amide | 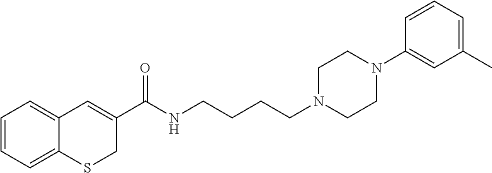 |
| 114 | 2H-thiochromene-3- carboxylic acid {4-[4-(2- chloro-phenyl)-piperazin- 1-yl]-butyl}-amide | 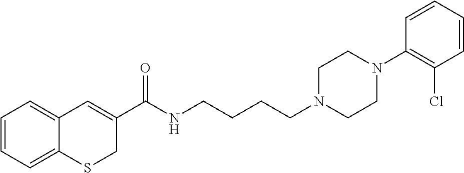 |
| 115 | 2H-thiochromene-3- carboxylic acid {4-[4-(3- chloro-phenyl)-piperazin- 1-yl]-butyl}-amide | 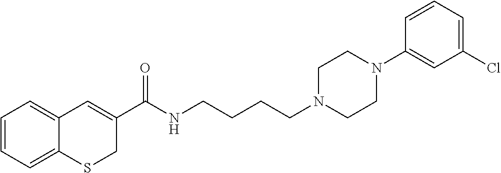 |
| 116 | 2H-thiochromene-3- carboxylic acid {4-[4-(4- chloro-phenyl)-piperazin- 1-yl]-butyl}-amide | 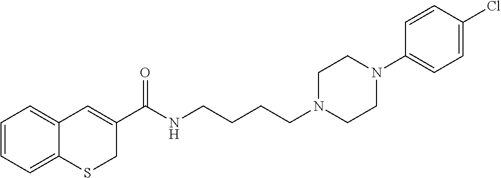 |
| 117 | 2H-thiochromene-3- carboxylic acid {4-[4-(2,4- dimethoxy-phenyl)- piperazin-1-yl]-butyl}- amide | 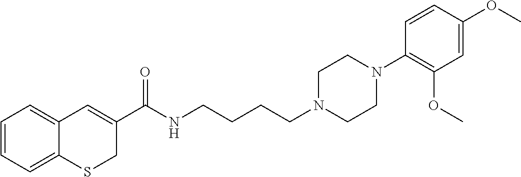 |
| 118 | 2H-thiochromene-3- carboxylic acid {4-[4-(3,4- dimethoxy-phenyl)- piperazin-1-yl]-butyl}- amide | 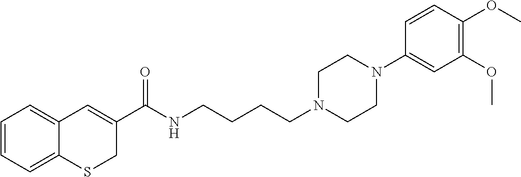 |
| 119 | 2H-thiochramene-3- carboxylic acid {4-[4-(3- formyl-phenyl)-piperazin- 1-yl]-butyl}-amide | 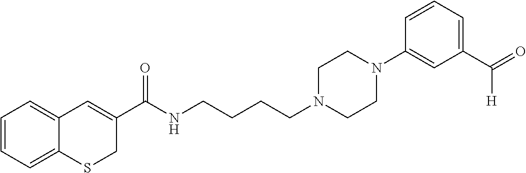 |
| 120 | 2H-thiochromene-3- carboxylic acid {4-[4-(3- nitro-phenyl)-piperazin-1- yl]-butyl}-amide | 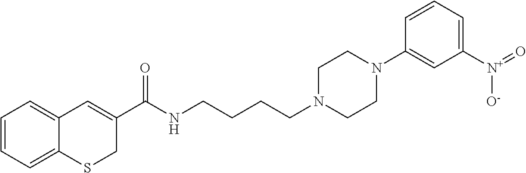 |
| 121 | 5-(4-{4-[2H- thiochromene-3- carbonyl)-amino]-butyl)- piperazin-1-yl)- benzofuran-2-carboxylic acid methyl ester | 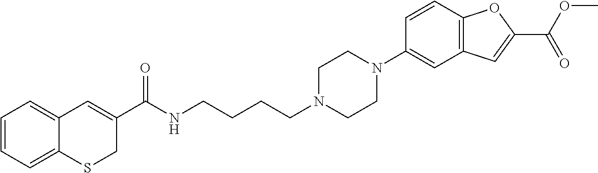 |
| 122 | 2H-thiochromene-3- carboxylic acid {4-[4-(2,3- dihydro-benzo[1,4]dioxin- 6-yl)-piperazin-1-yl]- butyl}-amide | 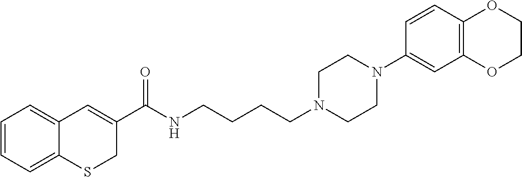 |
| 123 | 2H-thiochromene-3- carboxylic acid {4-[4-(3- acetylamino-phenyl)- piperazin-1-yl]-butyl}- amide | 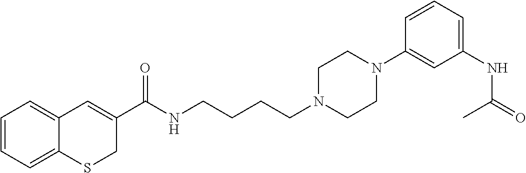 |
| 124 | 2H-thiochromene-3- carboxylic acid {4-[4-(3- hydroxymethyl)-piperazin- 1-yl]-butyl}-amide | 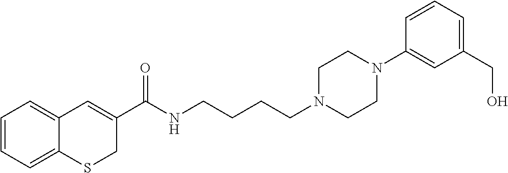 |
| 125 | 2H-thiochromene-3- carboxylic acid {4-[4-(3- methyloxy-carbonyl)- piperazin-1-yl]-butyl}- amide | 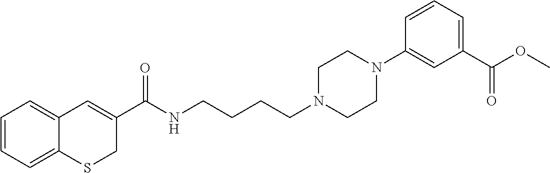 |
| 126 | 2H-thiochromene-3- carboxylic acid {4-[4-(2,3- dihydro-benzo[1,4]dioxin- 5-yl)-piperazin-1-yl]- butyl}-amide | 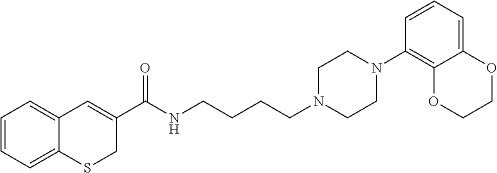 |
| 127 | 6-chloro-2H- thiochromene-3-carboxylic acid {4-[4-(2,3-dihydro- benzo[1,4]dioxin-6-yl)- piperazin-1-yl]-butyl}- amide | 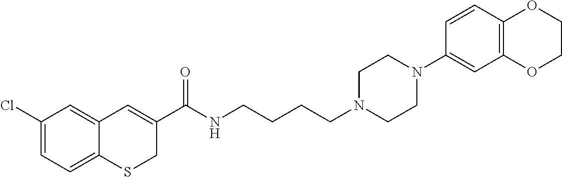 |
| 128 | 6-chloro-2H- thiochromene-3-carboxylic acid {4-[4-(3-cyano- phenyl)-piperazin-1-yl]- butyl}-amide | 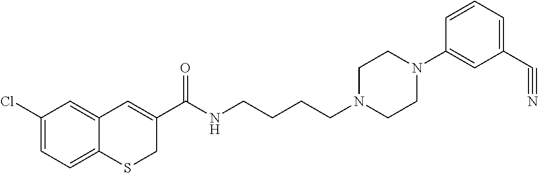 |
| 129 | 6-chloro-2H- thiochromene-3-carboxylic acid {4-[4-(3-hydroxy- phenyl)-piperazin-1-yl]- butyl}-amide | 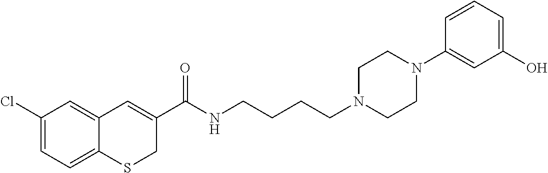 |
| 130 | 6-chloro-2H- thiochromene-3-carboxylic acid {4-[4-(2-methoxy- phenyl)-piperazin-1-yl]- butyl}-amide | 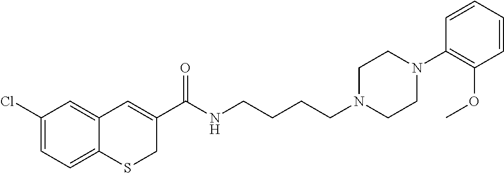 |
| 131 | 6-chloro-2H- thiochromene-3-carboxylic acid {4-[4-(2-fluoro- phenyl)-piperazin-1-yl]- butyl}-amide | 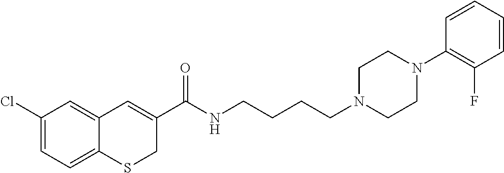 |
| 132 | 6-chloro-2H- thiochromene-3-carboxylic acid {4-[4-(2,4- dimethoxy-phenyl)- piperazin-1-yl]-butyl}- amide | 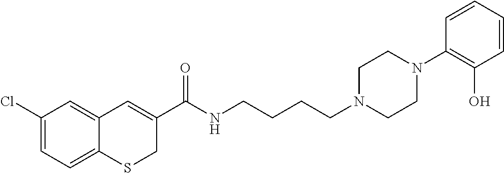 |

| | Empirical | | | | |
|---|---|---|---|---|---|
| Example | formula | | Mass | NMR | Synthesis |
| No. | (base) | Salt | (MH⁺) | ¹H (δ in ppm) | method |
| 65 | C₂₆H₃₃O₄N₃ MW = 451.57 | HCl | 452.3 | (CD₃OD): 3.78 (s, 3 H, OCH₃ chrom.), 3.86 (s, 3 H, OCH₃) | Example 1 |
| 66 | C₂₆H₃₀O₃N₄ MW = 446.55 | HCl | 447.3 | (CD₃OD): 3.78 (s, 3 H, OCH₃ chrom.), 7.09- 7.47 (m, 7 H, H arom) | Example 1 |
| 67 | C₂₅H₂₀O₃N₃Cl₂ MW = 490.43 | HCl | 490.2 | (CD₃OD): 3.78 (s, 3 H, OCH₃ chrom.), 6.42-7.3 (m, 7 H, H arom + H₄) | Example 1 |
| 68 | C₂₅H₃₁O₄N₃ MW = 437.54 | HCl | 438.3 | (CD₃OD): 3.78 (s, 3 H, OCH₃ chrom.), 6.38- 7.19 (m, 8 H, H arom + H₄) | Example 41 |
| 69 | C₂₇H₃₃O₅N₃ MW = 479.58 | HCl | 480.2 | (DMSO): 3.78 (s, 3 H, OCH₃ chrom.), 4.16- 4.21 (m, 2 H + 2 H, OCH₂—CH₂O—), 6.45 7.24 (m, 7 H, H arom + H₄) | Example 41 |
| 70 | C₂₇H₃₃O₅N₃ MW = 479.58 | HCl | 480.1 | (DMSO): 3.78 (s, 3 H, OCH₃ chrom.), 4.16- 4.21 (m, 2 H + 2 H, OCH₂—CH₂O—), 6.45- 7.24 (m, 7 H, H arom + H₄) | Example 41 |
| 71 | C₂₅H₂₉Cl₂O₃N₃ MW = 490.43 | HCl | 490.2 | (CD₃OD): 3.75 (s, 3H, OCH₃ chrom), 6.75- 7.69 (m, 7 H, H arom + H₄) | Example 1 |
| 72 | C₂₈H₃₅O₅N₃ MW = 493.61 | HCl | 494.3 | (CD₃OD): 1.38 (t, 3 H, COOCH₂—CH₃), 3.75 (s, 3 H, OCH₃ chrom), 4.37 (q, 2 H, COOCH₂—CH₃) | Example 41 |
| 73 | C₂₅H₃₂O₃N₄ MW = 436.56 | HCl | 437.3 | (CD₃OD): 3.75 (s, 3 H, OCH₃ chrom), 6.36- 7.18 (m, 8 H, H arom + H₄) | Example 41 |
| 74 | C₂₅H₃₀O₅N₄ MW = 466.55 | HCl | 467.2 | (CD₃OD): 3.75 (s, 3 H, OCH₃ chrom), 6.76- 7.85 (m, 7 H, H arom + H₄) | Example 41 |
| 75 | C₂₆H₃₃O₄N₃ MW = 451.57 | HCl | 452.3 | (CD₃OD): 3.75 (s, 3 H, OCH₃ chrom), 4.58 (s, 2 H, Ar—CH₂—OH), 6.76- 7.85 (m, 8 H, H arom + H₄) | Example 41 |
| 76 | C₂₇H₃₄O₄N₄ MW = 478.60 | HCl | 479.3 | (CD₃OD): 2.18 (s, 3 H, CH₃—CO—NH—), 3.75 (s, 3 H, OCH₃ chrom), | Example 41 |
| 77 | C₂₅H₃₁O₄N₃ MW = 437.54 | HCl | 438.2 | (CD₃OD): 3.75 (s, 3H, OCH₃ chrom), 6.36- 7.45 (m, 7 H, H arom + H₄) | Example 41 |
| 78 | C₂₇H₃₃O₅N₃ MW = 479.58 | HCl | 480.1 | (DMSO): 3.78 (s, 3 H, OCH₃ chrom.), 4.16- 4.83 (m, 2 H + 2 H, OCH₂—CH₂O—), 6.50- 7.46 (m, 7 H, H arom + H₄) | Example 41 |
| 79 | C₂₇H₃₃O₅N₃ MW = 479.58 | HCl | 480.2 | (DMSO): 3.72 (s, 3 H, OCH₃ chrom.), 4.16- 4.21 (m, 2 H + 2 H, OCH₂—CH₂O—), 6.49- 7.24 (m, 7 H, H arom + H₄) | Example 41 |
| 80 | C₂₇H₃₄O₄N₄ MW = 478.60 | HCl | 479.2 | (DMSO): 2.78 (s, 3 H, CH₃—NH—CO—), 3.72 (s, 3 H, OCH₃ chrom.) | Example 41 |
| 81 | C₂₅H₃₄N₄O₅S MW = 514.65 | base | 515.2 | (CDCl3): 2.98 (s, 3 H, CH₃—SO₂—), 3.74 (s, 3 H, OMe), 6.53-7.22 (m, 8 H, H₄ + Haro) | Example 1 |
| 82 | C₂₇H₃₃O₅N₃ MW = 479.58 | HCl | 480.2 | (DMSO): 3.50 (s, 3 H, CH₃—O—CO—), 3.72 (s, 3 H, OCH₃ chrom.) | Example 41 |
| 83 | C₂₄H₂₆O₂N₃Cl₃ MW = 494.85 | HCl | 494.2 | (CD₃OD): 6.83-7.82 (m, 7 H, H arom + H₄) | Example 1 |
| 84 | C₂₄H₂₇O₄N₄Cl MW = 470.97 | HCl | 471.2 | (CD₃OD): 6.82 (d, 1 H, H₄), 7.15-7.20 (m, 3 H, H arom Chrom.), 7.44- 8.08 (m, 4 H, H arom. Aryl-NO₂) | Example 41 |
| 85 | C₂₄H₂₉O₂N₄Cl MW = 440.98 | HCl | 441.2 | (CD₃OD): 6.82 (d, 1 H, H₄), 6.90-6.99 (m, 2 H, Aryl-NH₂), 7.15-7.20 (m, 3 H, H arom Chrom.), 7.44-8.08 (m, 2 H, Aryl-NH₂) | Example 41 |
| 86 | C₂₆H₃₁O₃N₄Cl MW = 483.01 | HCl | 483.2 | (CD₃OD): 2.11 (s, 3 H, CH₃—CO—NH—), 6.75- 7.71 (m, 8 H, H arom + H₄) | Example 41 |
| 87 | C₂₄H₂₈O₃N₃Cl MW = 441.96 | HCl | 442.2 | (CD₃OD): 6.37-6.50 (m, 3 H, Aryl-OH), 6.82 (d, 1 H, H₄), 7.08-7.37 (m, 4 H, Aryl-OH + chrom.) | Example 41 |
| 88 | C₂₅H₃₀O₃N₃Cl MW = 455.99 | HCl | 456.2 | (CD₃OD): 4.58 (s, 2 H, Aryl-CH₂—OH), 6.81 (d, 1 H, H₄), 6.93-7.92 (m, 7 H, Haro) | Example 41 |
| 89 | C₂₅H₃₁O₄N₄SCl MW = 519.07 | HCl | 519.2 | (CD₃OD): 3.1 (s, 3 H, CH₃—SO₂—), 6.80-7.92 (m, 8 H, H₄ + Haro) | Example 41 |
| 90 | C₂₄H₂₆O₂FN₃Cl₂ MW = 478.40 | HCl | 478.2 | (CD₃OD): 4.58 (s, 2 H, Aryl-CH₂—OH), 6.81 (d, 1 K, H₄), 6.93-7.92 (m, 7 H, Haro) | Example 1 |
| 91 | C₂₅H₃₀O₃FN₃ MW = 439.53 | HCl | 440.2 | (CD₃OD): 3.86 (s, 3 H, Aryl-OCH₃), 6.81-7.16 (m, H₄ + 8 H, Haro) | Example 1 |
| 92 | C₂₅H₂₇O₂FN₄ MW = 434.52 | HCl | 435.2 | (CD₃OD): 6.83 (m, 1 H, H₄), 6.97 (m, 2 H, Haro), 7.17-7.53 (m, 5 H, Haro.) | Example 41 |
| 93 | C₂₆H₃₁O₃FN₄ MW = 466.56 | HCl | 467.2 | (CD₃OD): 2.12 (s, 3 H, CH₃—CO—NH—), 6.76- 7.66 (m, 8 H, H arom + H₄) | |
| 94 | C₂₄H₂₈O₃FN₃ MW = 425.51 | HCl | 426.2 | (CD₃OD): 6.37-6.50 (m, 3 H, Aryl-OH), 6.82 (d, 1 H, H₄), 7.08-7.37 (m, 4 H, Aryl-OH + chrom.) | Example 41 |
| 95 | C₂₄H₂₇O₄FN₄ MW = 454.51 | HCl | 455.3 | (CD₃OD): 6.42-6.53 (m, 3 H, H arom. + H₄), 6.83 (m, 1 H, H arom), 6.93- 7.18 (m, 4 H, H arom. Aryl-NO₂ + H arom.) | Example 41 |
| 96 | C₂₅H₃₁O₄FSN₄ MW = 502.61 | HCl | 503.2 | (CD₃OD): 2.94 (s, 3 H, CH₃—SO₂—), 6.77-7.24 (m, 8 H, H₄ + Haro) | Example 41 |
| 97 | C₂₄H₂₉O₂FN₄ MW = 424.52 | HCl | 425.2 | (CD₃OD): 6.83 (m, 1 H, H₄), 6.90-7.00 (m, 4 H, Aryl-NH₂), 7.13-7.46 (m, 3 H, H arom Chrom.), | Example 41 |
| 98 | C₂₆H₃₀O₄FN₃ MW = 467.55 | HCl | 468.2 | (DMSO): 4.05-4.24 (m, 2 H + 2 H, OCH₂—CH₂O—), 6.51-7.24 (m, 7 H, H arom + H₄) | Example 41 |
| 99 | C₂₆H₃₀O₄FN₃ MW = 467.55 | HCl | 468.2 | (DMSO): 3.56 (s, 3 H, CH₃—O—CO—), 6.87- 7.52 (m, 7 H, H arom + H₄) | Example 41 |
| 100 | C₂₈H₃₀O₅FN₃ MW = 507.55 | HCl | 508.2 | (DMSO): 3.88 (s, 3 H, CH₂—O—CO—), 6.88- 7.66 (m, 8 H, H arom + H benzofurane. + H₄) | Example 41 |
| 101 | C₂₇H₃₅O₅N₃ MW = 481.60 | HCl | 482.3 | (DMSO): 3.57 (s, 3 H, Ar-OCH₃), 3.76 (s, 6 H, Ar-(OCH₃)₂), 6.26 (s, 2 H, H arom.), 6.83-7.50 (m, 5 H, H arom. + H₄) | Example 41 |
| 102 | C₂₆H₃₀O₂N₄ MW = 430.55 | HCl | 431.4 | (DMSO): 6.45-7.29 (m, 8 H, H arom. + H₄), 8.36 (m, 1 H, —CONH—), 11.16 (s, 1 H, NHindole) | Example 41 |
| 103 | C₂₄H₃₀O₂N₄ MW = 406.53 | HCl | 407.1 | (CD₃OD): 6.80 (d, 1 H, H4), 6.91-7.11 (m, 4 H, Aryl-NH₂), 7.14-7.35 (m, 4 H, H arom Chrom.), | Example 41 |
| 104 | C₂₆H₃₁O₄N₃ MW = 449.55 | HCl | 450.1 | (DMSO): 4.16-4.21 (m, 2 H + 2 H, OCH₂—CH₂O—), 6.49-7.25 (m, 8 H, H arom + H₄) | Example 41 |
| 105 | C₂₆H₃₁O₄N₃ MW = 449.55 | HCl | 450.1 | (DMSO): 4.23 (m, 2 H + 2 H, OCH₂—CH₂O—), 6.51-7.28 (m, 9 H, H arom + H₄) | Example 41 |
| 106 | C₂₈H₃₁O₅N₃ MW = 489.58 | HCl | 490.1 | (DMSO): 3.82 (s, 3 H, CH₃—O—OC—), 6.84- 7.67 (m, 9 H, H arom + H benzofurane. + H₄) | Example 41 |
| 107 | C₂₆H₃₂O₂N₄ MW = 432.57 | HCl | 433.2 | (DMSO): 3.10 (t, 2 H, Ar—CH₂—CH₂—N), 3.67 (t, 2 H, Ar—CH₂—CH₂—N.), 3.8 (m, Ar—CH₂—CH₂—NH—) | Example 41 |
| 108 | C₂₅H₃₂O₄SN₄ MW = 484.62 | HCl | 485.2 | (DMSO): 2.97 (s, 3 H, CH₃—SO₂—NH—Ar), 9.66 (s, 1 H, CH₃—SO₂—NH—Ar) | Example 41 |
| 109 | C₂₈H₃₄O₃N₄ MW = 474.61 | base | 475.3 | (DMSO): 2.13 (s, 3 H, CH₃—CO—), 3.01 (t, 2 H, Ar—CH₂—CH₂—N), 4.05 (t, 2 H, Ar—CH₂—CH₂—N.) | Example 41 |
| 110 | C₂₅H₂₈O₄N₄ MW = 448.53 | base | 449.2 | (DMSO): 6.59-8.22 (m, 7 H, Haro), 11.51 (s, Ar—O—CO—NH—Ar) | Example 41 |
| 111 | C₂₆H₃₁O₃N₃ MW = 433.56 | HCl | 434.3 | (DMSO): 3.2 (t, 2 H, Ar—CH₂—CH₂—O—Ar.), 4.53 (t, 2 H, Ar—CH₂—CH₂—O—Ar) | Example 1 |
| 112 | C₂₆H₃₃OSN₃ MW = 435.64 | HCl | 436.3 | (CD₃OD): 2.25 (d, 6 H, Ar—(CH₃)₂), 6.94-7.39 (m, 8 H, Haro + H₄) | Example 41 |
| 113 | C₂₅H₃₁OSN₃ MW = 421.61 | HCl | 422.3 | (CD₃OD): 2.30 (s, 3 H, Ar—CH₃), 6.75-7.38 (m, 9 H, Haro + H₄) | Example 1 |
| 114 | C₂₄OH₂₈ClSN₃ MW = 442.03 | HCl | 442.2 | (CO₃OD): 7.08-7.31 (m, 8 H, Haro + H₄), 7.39- 7.43 (d, 1 H, Haro) | Example 41 |
| 115 | C₂₄H₂₈OClSN₃ MW = 442.03 | HCl | 442.2 | (CD₃OD): 6.90-6.95 (m, 2 H, Haro + H₄), 7.03 (s, 1 H, Haro Ar—Cl), 7.13- 7.41 (m, 6 H, Haro) | Example 41 |
| 116 | C₂₄H₂₈OClSN₃ MW = 442.03 | HCl | 442.2 | (CD₃OD): 6.96-7.06 (d, 2 H, Haro), 7.13-7.38 (m, 7 H, Haro + H₄) | Example 41 |
| 117 | C₂₆H₃₃O₃SN₃ MW = 467.64 | HCl | 468.3 | (CD₃OD): 3.77 (s, 3 H, Ar—OCH₃), 3.82 (s, 3 H, Ar—OCH₃) 6.46-7.27 (m, 8 H, Haro + H₄) | Example 41 |
| 118 | C₂₆H₃₃O₃SN₃ MW = 467.64 | HCl | 468.3 | (CD₃OD): 3.76 (s, 3 H, Ar—OCH₃), 3.84 (s, 3 H, Ar—OCH₃), 6.52-7.38 (m, 8 H, Haro + H₄) | Example 41 |
| 119 | C₂₅H₂₉O₂SN₃ MW = 435.59 | HCl | 436.5 | (CD₃OD): 7.00-7.32 (m, 9 H, Haro + H₄), 9.95 (s, 1 H, Ar—CHO) | Example 41 |
| 120 | C₂₄H₂₈O₃SN₄ MW = 451.58 | HCl | 453.3 | (CD₃OD): 7.13-7.83 (m, 9 H, Haro + H₄) | Example 41 |
| 121 | C₂₈H₃₁SO₄N₃ MW = 505.64 | HCl | 506.1 | (DMSO): 3.87 (s, 3 H, CH₃—O—OC—), 7.18- 7.66 (m, 8 H, H arom + H benzofurane. + H₄) | Example 41 |
| 122 | C₂₆H₃₁O₃SN₃ MW = 465.62 | HCl | 466.1 | (DMSO): 4.16-4.20 (m, 2 H + 2 H, OCH₂—CH₂O—), 6.48-7.31 (m, 8 H, H arom + H₄) | Example 41 |
| 123 | C₂₆H₃₂O₂SN₄ MW = 464.63 | HCl | 465.2 | (CD₃OD): 2.11 (s, 3 H, CH₃—CO—NH—), 6.93- 7.79 (m, 9 H, H arom + H₄) | Example 41 |
| 124 | C₂₅H₃₁O₂SN₃ MW = 437.61 | HCl | 438.2 | (CD₃OD): 4.56 (s, 2 H, Aryl-CH₂—OH), 6.83 (d, 1 H, H₄), 6.93-7.89 (m, 8 H, Haro) | Example 41 |
| 125 | C₂₆H₃₁O₃SN₃ MW = 465.62 | HCl | 466.1 | (DMSO): 3.67 (s, 3 H, CH₃—O—CO—), 7.15- 7.64 (m, 9 H, H arom + H₄) | Example 41 |
| 126 | C₂₆H₃₁O₃SN₃ MW = 465.62 | HCl | 466.2 | (DMSO): 4.22-4.24 (m, 2 H + 2 H, OCH₂—CH₂O—), 6.50-7.32 (m, 8H, H arom + H₄) | Example 41 |
| 127 | C₂₈H₃₀O₃SClN₃ MW = 500.06 | HCl | 500.1 | (DMSO): 4.19-4.21 (m, 2 H + 2 H, OCH₂—CH₂O—), 6.48-7.41 (m, 7 H, H arom + H₄) | Example 41 |
| 128 | C₂₅H₂₇OSClN₄ MW = 467.04 | HCl | 467.1 | (DMSO): 7.25-7.62 (m, 8 H, H arom + H₄) (DMSO): 6.29-7.42 (m, 8 H, H arom + H₄), 9.29 (s, 1 H, Ar—OH) | Example 41 |
| 129 | C₂₄H₂₈O₂SClN₃ MW = 458.03 | HCl | 458.1 | (DMSO): 6.29-7.42 (m, 8 H, H arom + H₄), 9.29 (s, 1 H, Ar—OH) | Example 41 |
| 130 | C₂₄H₃₀O₂SClN₃ MW = 472.05 | HCl | 472.1 | (DMSO): 3.79 (s, 3 H, Ar—OCH₃), 6.91-7.42 (m, 8 H, Haro + H₄) | Example 41 |
| 131 | C₂₄H₂₇OFSClN₃ MW = 460.02 | HCl | 460.1 | (DMSO): 7.05-7.42 (m, 8 H, Haro + H₄) | Example 41 |
| 132 | C₂₄H₂₈O₂SClN₃ MW = 458.03 | HCl | 458.1 | (DMSO): 6.76-7.61 (m, 8 H, H arom + H₄), 9.30 (s, 1 H, Ar—OH) | Example 41 |

The binding inhibition constants for certain compounds according to the invention, expressed in pKi, on dopamine D2 and D3 receptors and on α₁-adrenergic receptor are presented in table 2 below.

TABLE 2

| Example | | | | Intrinsic |
|---|---|---|---|---|
| no. | pKi D3⁽¹⁾ | pKi D2⁽¹⁾ | pKi α₁⁽²⁾ | activity |
| 2 | 9.09 | 6.91 | 7.29 | nt⁽³⁾ |
| 55 | 9.10 | 6.6 | 7.13 | 0 |
| 54 | 9.27 | 6.38 | 8.05 | nt |
| 102 | 9.66 | 7.03 | 8.05 | 0.59 |
| 35 | 8.62 | 5.5 | 7.32 | 0.18 |
| 89 | 9.66 | 7.46 | 6.78 | 0 |
| 111 | 9.17 | 6.85 | 7.84 | 0.26 |
| 110 | 10.17 | 8.12 | 8.44 | 0.81 |
| 105 | 9.22 | 6.88 | 7.81 | nt |
| 41 | 9.18 | 5.42 | 7.08 | 0.64 |
| 16 | 8.57 | 6.35 | 7.55 | nt |

⁽¹⁾Measured by inhibition of spiperone[³H] binding in CHO cells as described by Cussac et al. in Naunyn-Schmiedeberg's Arch. Pharmacol. 2000, 361, 569.
⁽²⁾Measured by inhibition of prazocine[³H] binding in rat brain tissue as described in Hornung et al. in Naunyn-Schmiedeberg's Arch Pharmacol 1979, 308, 223.
⁽³⁾Not tested

1. A compound of general formula 1 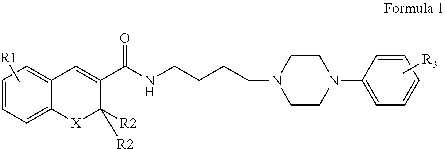
wherein:
X represents a heteroatom, O or S;
R1 represents an atom of hydrogen or one or more identical or different substituents on the homocycle consisting of halogen, Cl, F, Br or a C₁₋₄ alkoxy, OH, C₁₋₄ alkyl or CF₃ group;
R2 represents a hydrogen atom or C₁₋₄ alkyl group;
R3 represents a hydrogen atom or one or more identical or different substituents consisting of halogen, Cl, F, Br or a C₁₋₄ alkyl, C₁₋₄ alkoxy or thioalkoxy, O(CH₂)ₙO with n being 1 or 2, NO₂, NH₁₂, NCOCH₃, NHSO₂CH₃, OH, CF₃, CN, COOEt or CH₂OH group, a phenyl or benzyl substituent optionally substituted, or R3 forms a ring fused with the aromatic ring which carries it.

2. A compound according to claim 1, wherein said compound is selected among the group comprising:
2H-Chromene-3-carboxylic acid {4-[4-(2-methoxyphenyl)-piperazin-1-yl]-butyl}-amide
2H-Chromene-3-carboxylic acid {4-[4-(4-methoxyphenyl)-piperazin-1-yl]-butyl}-amide
2H-Chromene-3-carboxylic acid {4-[4-(2-fluorophenyl)-piperazin-1-yl]-butyl}-amide
2H Chromene-3-carboxylic acid {4-[4-(4-fluorophenyl)-piperazin-1-yl]-butyl}-amide
2H-Chromene-3-carboxylic acid {4-[4-phenylpiperazin-1-yl]-butyl}-amide
2H-Chromene-3-carboxylic acid {4-[4-(2-chlorophenyl)-piperazin-1-yl]-butyl}-amide
2H-Chromene-3-carboxylic acid {4-[4-(4-chlorophenyl)-piperazin-1-yl]-butyl}-amide
2H-Chromene-3-carboxylic acid {4-[4-(2,3-dichlorophenyl)-piperazin-1-yl]-butyl}-amide
2H-Chromene-3-carboxylic acid {4-[4-(3-chlorophenyl)-piperazin-1-yl]-butyl}-amide
2H-Chromene-3-carboxylic acid {4-[4-(3-trifluoromethylphenyl)piperazin-1-yl]-butyl}-amide
2H-Chromene-3-carboxylic acid {4-[4-(2-trifluoromethylphenyl)-piperazin-1-yl]-butyl}-amide
2H-Chromene-3-carboxylic acid {4-[4-(4-trifluoromethylphenyl)piperazin-1-yl]-butyl}-amide
2H-Chromene-3-carboxylic acid {4-[4-(4-nitrophenyl)-piperazin-1-yl]-butyl}-amide
2H-Chromene-3-carboxylic acid {4-[4-(3-nitrophenyl)-piperazin-1-yl]-butyl}-amide
2H-Chromene-3-carboxylic acid {4-[4-(3-aminophenyl)-piperazin-1-yl]-butyl}-amide
2H-Chromene-3-carboxylic acid {4-[4-(3-acetamidophenyl)-piperazin-1-yl]-butyl}-amide
2H-Chromene-3-carboxylic acid {4-[4-(3-methylsulfonamidophenyl)-piperazin-1-yl]-butyl}-amide
2H-Chromene-3-carboxylic acid {4-[4-(2-nitrophenyl)-piperazin-1-yl]-butyl}-amide
2H-Chromene-3-carboxylic acid {4-[4-(2,3-dimethylphenyl)-piperazin-1-yl]-butyl}-amide
2H-Chromene-3-carboxylic acid {4-[4-(3,4-dimethylphenyl)-piperazin-1-yl]-butyl}-amide
2H-Chromene-3-carboxylic acid {4-[4-(2,4-dimethylphenyl)piperazin-1-yl]-butyl}-amide
2H-Chromene-3-carboxylic acid {4-[4-(2-methylphenyl)-piperazin-1-yl]-butyl}-amide
2H-Chromene-3-carboxylic acid {4-[4-(3-methoxyphenyl)-piperazin-1-yl]-butyl}-amide
2H-Chromene-3-carboxylic acid {4-[4-(2-hydroxyphenyl)-piperazin-1-yl]-butyl}-amide
2H-Chromene-3-carboxylic acid {4-[4-(3-hydroxyphenyl)piperazin-1-yl]-butyl}-amide
2H-Chromene-3-carboxylic acid {4-[4-(4-hydroxyphenyl)-piperazin-1-yl]-butyl}-amide
2H-Chromene-3-carboxylic acid {4-[4-(3,4-methylenedioxyphenyl)-piperazin-1-yl]-butyl}-amide
2H-Chromene-3-carboxylic acid {4-[4-(3,4-dimethoxyphenyl)-piperazin-1-yl]-butyl}-amide
2H-Chromene-3-carboxylic acid {4-[4-(3,5-dimethoxyphenyl)-piperazin-1-yl]-butyl}-amide
2H-Chromene 3 carboxylic acid {4-[4-(2-cyanophenyl)-piperazin-1-yl]-butyl}-amide
2H-Chromene-3-carboxylic acid {4-[4-(3-cyanophenyl)-piperazin-1-yl]-butyl}-amide
2H-Chromene-3-carboxylic acid {4-[4-(4-cyanophenyl)-piperazin-1-yl]-butyl}-amide
2H-Chromene-3-carboxylic acid {4-[4-(3-ethoxycarbonylphenyl)-piperazin-1-yl]-butyl}-amide
2H-Chromene-3-carboxylic acid {4-[4-(4-ethoxycarbonylphenyl)-piperazin-1-yl]-butyl}-amide
2H-Chromene-3-carboxylic acid {4-[4-(3-hydroxymethylphenyl)-piperazin-1-yl]-butyl}-amide
6-Methoxy-2H-chromene-3-carboxylic acid {4-[4-(2-methoxyphenyl)-piperazin-1-yl]-butyl}-amide
6-Methoxy-2H-chromene-3-carboxylic acid {4-[4-(2-fluorophenyl)-piperazin-1-yl]butyl}-amide
6-Methoxy-2H-chromene-3-carboxylic acid {4-[4-(2,3-dichlorophenyl)-piperazin-1-yl]-butyl}-amide
6-Methoxy-2H-chromene-3-carboxylic acid {4-[4-(3-methoxyphenyl)-piperazin-1-yl]-butyl}-amide
6-Methoxy-2H-chromene-3-carboxylic acid {4-[4-(4-methoxyphenyl)-piperazin-1-yl]-butyl}-amide
6-Methoxy-2H-chromene-3-carboxylic acid {4-[4-(3-hydroxyphenyl)-piperazin-1-yl]-butyl}-amide
6-Methoxy-2H-chromene-3-carboxylic acid {4-[4-(3-cyanophenyl)-piperazin-1-yl]-butyl}-amide
6-Methoxy-2H-chromene-3-carboxylic acid {4-[4-(cyanophenyl)piperazin-1-yl]-butyl}-amide
6-Methoxy-2H-chromene-3-carboxylic acid {4-[4-(2-cyanophenyl)piperazin-1-yl]-butyl}-amide
6-Methoxy-2H-chromene-3-carboxylic acid {4-[4-(3-ethoxycarbonylphenyl)-piperazin-1-yl]-butyl}-amide
6-Methoxy-2H-chromene-3-carboxylic acid {4-[4-(3-hydroxymethylphenyl)-piperazin-1-yl]-butyl}-amide
6-Methoxy-2H-chromene-3-carboxylic acid {4-[4-(3,4-dimethoxyphenyl)piperazin-1-yl]-butyl}-amide
6-Methoxy-2H-chromene-3-carboxylic acid {4-[4-(3,4-methylenedioxyphenyl)-piperazin-1-yl]-butyl}-amide
2,2-Dimethyl-2H-chromene-3-carboxylic acid {4-[4-(3-hydroxyphenyl)-piperazin-1-yl]-butyl}-amide
2,2-Dimethyl-2H-chromene-3-carboxylic acid {4-[4-(2-cyanophenyl)-piperazin-1-yl]-butyl}-amide
2,2-Dimethyl-2H-chromene-3-carboxylic acid {4-[4-(3-cyanophenyl)-piperazin-1-yl]-butyl}-amide
2,2-Dimethyl-2H-chromene-3-carboxylic acid {4-[4-(3-hydroxymethylphenyl)-piperazin-1-yl]-butyl}-amide
2H-Thiochromene-3 carboxylic acid {4-[4-(2-methoxyphenyl)-piperazin-1-yl]-butyl}-amide
2H-Thiochromene-3-carboxylic acid {4-[4-(2-fluorophenyl)-piperazin-1-yl]-butyl}-amide
2H-Thiochromene-3-carboxylic acid {4-[4-(2-chlorophenyl)-piperazin-1-yl]-butyl}-amide
2H-Thiochromene-3-carboxylic acid {4-[4-(2,3-dichlorophenyl)piperazin-1-yl]-butyl}-amide
2H-Thiochromene-3-carboxylic acid {4-[4-(3-chlorophenyl)-piperazin-1-yl]-butyl}-amide
2H-Thiochromene-3-carboxylic acid {4-[4-(3-trifluoromethylphenyl)-piperazin-1-yl]-butyl}-amide
2H-Thiochromene-3-carboxylic acid {4-[4-(3-methoxyphenyl)-piperazin-1-yl]-butyl}-amide
2H-Thiochromene-3-carboxylic acid {4-[4-(4-methoxyphenyl)-piperazin-1-yl]-butyl}-amide
2H-Thiochromene-3-carboxylic acid {4-[4-(3,4-dimethoxyphenyl)-piperazin-1-yl]-butyl}-amide
2H-Thiochromene-3-carboxylic acid {4-[4-(3-hydroxyphenyl)-piperazin-1-yl]-butyl}-amide
2H-Thiochromene-3-carboxylic acid {4-[4-(2-hydroxyphenyl)-piperazin-1-yl]-butyl}-amide
2H-Thiochromene-3-carboxylic acid {4-[4-(4-hydroxyphenyl)piperazin-1-yl]-butyl}-amide
2H-Thiochromene-3-carboxylic acid {4-[4-(2-cyanophenyl)-piperazin-1-yl]-butyl}-amide
2H-Thiochromene-3-carboxylic acid {4-[4-(4-cyanophenyl)-piperazin-1-yl]-butyl}-amide
2H-Thiochromene-3-carboxylic acid {4-[4-(3-cyanophenyl)-piperazin-1-yl]-butyl}-amide
2H-Thiochromene-3-carboxylic acid {4-[4-(3-methoxycarbonylphenyl)-piperazin-1-yl]-butyl}-amide
2H-Thiochromene-3-carboxylic acid {4-[4-(3-hydroxymethylphenyl)piperazin-1-yl]-butyl}-amide
6-Methoxy-2H-thiochromene-3-carboxylic acid {4-[4-(2-methoxyphenyl)-piperazin-1-yl]-butyl}-amide
6-Methoxy-2H-thiochromene-3-carboxylic acid {4-[4-(2-fluorophenyl)-piperazin-1-yl]-butyl}-amide
6-Methoxy-2H-thiochromene-3-carboxylic acid {4-[4-(3-chlorophenyl)-piperazin-1-yl]-butyl}-amide
6-Methoxy-2H-thiochromene-3-carboxylic acid {4-[4-(3-trifluoromethylphenyl)-piperazin-1-yl]-butyl}-amide
6-Methoxy-2H-thiochromene-3-carboxylic acid {4-[4-(3 methoxyphenyl)-piperazin-1-yl]-butyl}-amide
6-Methoxy-2H-thiochromene-3-carboxylic acid {4-[4-(3,4-dimethoxyphenyl)-piperazin-1-yl]-butyl}-amide
6-Methoxy-2H-thiochromene-3-carboxylic acid {4-[4-(3-hydroxyphenyl)-piperazin-1-yl]-butyl}-amide
6-Methoxy-2H-thiochromene-3-carboxylic acid {4-[4-(3-cyanophenyl)-piperazin-1-yl]-butyl}-amide
6-Methoxy-2H-thiochromene-3-carboxylic acid {4-[4-(3-ethoxycarbonylphenyl)-piperazin-1-yl]-butyl}-amide
6 Methoxy-2H-thiochromene-3-carboxylic acid {4-[4-(3-hydroxymethylphenyl)-piperazin-1-yl]-butyl}-amide
2,2-Dimethyl-2H-thiochromene-3-carboxylic acid {4-[4-(2-cyanophenyl)piperazin-1-yl]-butyl}-amide
2,2-Dimethyl-2H-thiochromene-3-carboxylic acid {4-[4-(3-hydroxymethylphenyl)-piperazin 1-yl]-butyl}-amide
2,2-Dimethyl-2H-thiochromene-3-carboxylic acid {4-[4-(2-methoxyphenyl)-piperazin-1-yl]-butyl}-amide
2,2-Dimethyl-2H-thiochromene-3-carboxylic acid {4-[4-(2 fluorophenyl)-piperazin-1-yl]-butyl}-amide
2,2-Dimethyl-2H-thiochromene-3-carboxylic acid {4-[4-(2,3-dichlorophenyl)-piperazin-1-yl]-butyl}-amide
2,2-Dimethyl-2H-thiochromene-3-carboxylic acid {4-[4-(3-chlorophenyl)piperazin-1-yl]-butyl}-amide
2,2-Dimethyl-2H-thiochromene-3-carboxylic acid {4-[4-(3-hydroxyphenyl)piperazin-1-yl]-butyl}-amide
2,2-Dimethyl-2H-thiochromene-3-carboxylic acid {4-[4-(3-cyanophenyl)-piperazin-1-yl]-butyl}-amide
5-Bromo-8-methoxy-2H-chromene-3-carboxylic acid {4-[4-(2-methoxyphenyl)-piperazin-1-yl]-butyl}-amide
5-Bromo-8-methoxy-2H-chromene-3-carboxylic acid {4-[4-(2,3-dichlorophenyl)-piperazin-1-yl]-butyl}-amide
6-Chloro-2H-chromene-3-carboxylic acid {4-[4-(2-methoxyphenyl)-piperazin-1-yl]-butyl}-amide
6-Chloro-2H-chromene-3-carboxylic acid {4-[4-(2,3-dichloro-methoxyphenyl)-piperazin-1-yl]-butyl}-amide
6-Chloro-2H-chromene-3-carboxylic acid {4-[4-(2-fluorophenyl)piperazin-1-yl]-butyl}-amide
6-Chloro-2H-chromene-3-carboxylic acid {4-[4-(2-cyanophenyl)-piperazin-1-yl]-butyl}-amide
6 Chloro-2H-chromene-3-carboxylic acid {-4-[4-(3-cyanophenyl)-piperazin-1-yl]-butyl}-amide
6-Chloro-2H-chromene-3-carboxylic acid {4-[4-(cyanophenyl)-piperazin-1-yl]-butyl}-amide
6-Chloro-2H-chromene-3-carboxylic acid {4-[4-(3-hydroxyphenyl)-piperazin-1-yl]-butyl}-amide
6-Chloro-2H-chromene-3-carboxylic acid {4-[4-(3,4-dimethoxy-phenyl)-piperazin-1-yl]-butyl}-amide
6-Chloro-2H-chromene-3-carboxylic acid {4-[4-(3,4-methylenedioxy-phenyl)piperazin-1-yl]-butyl}-amide
7-Methoxy-2H-chromene-3-carboxylic acid {4-[4-(2-methoxy-phenyl)piperazin-1-yl]-butyl}-amide
7-Methoxy-2H-chromene-3-carboxylic acid {4-[4-(3-cyano-phenyl)piperazin-1-yl]-butyl}-amide
7-Methoxy-2H-chromene-3-carboxylic acid {4-[4-(2,3-dichloro-phenyl)piperazin-1-yl]-butyl}-amide
7-Methoxy-2H-chromene-3-carboxylic acid {4-[4-(3-hydroxy-phenyl)-piperazin-1-yl]-butyl}-amide
7-Methoxy-2H-chromene-3-carboxylic acid {4-[4-(2,3-dihydro-benzo[1,4]dioxin-6-yl)-piperazin-1-yl]-butyl}-amide
7-Methoxy-2H-chromene-3-carboxylic acid {4-[4-(3-methyloxy-carbonyl-)-piperazin-1-yl]-butyl}-amide
6-Methoxy-2H-chromene-3-carboxylic acid {4-[4-(2,4-dichloro-phenyl)-piperazin-1-yl]-butyl}-amide
6-Methoxy-2H-chromene-3-carboxylic acid {4-[4-(3-amino-phenyl)-piperazin-1-yl]-butyl}-amide
6-Methoxy-2H-chromene-3-carboxylic acid {4-[4-(3-nitro-phenyl)-piperazin-1-yl]-butyl}-amide
6-Methoxy-2H-chromene-3-carboxylic acid {4-[4-(3-acetylamino-phenyl)-piperazin-1-yl]-butyl}-amide
6-Methoxy-2H-chromene-3-carboxylic acid {4-[4-(2,3-benzo-1,4-dioxanyl-)-piperazin-1-yl]-butyl}-amide
6-Methoxy-2H-chromene-3-carboxylic acid {4-[4-(3,4-benzo-1,4-dioxanyl-)-piperazin-1-yl]-butyl}-amide
6-Methoxy-2H-chromene-3-carboxylic acid {4-[4-(2-oxo-2,3-dihydro-1H-benzimidazol-4-yl)-piperazin-1-yl]-butyl}-amide
6-Methoxy-2H-chromene-3-carboxylic acid {4-[4-(3H-benzimidazol-4-yl)-piperazin-1-yl]-butyl}-amide
6-Methoxy-2H-chromene-3-carboxylic acid {4-[4-(2-oxo-2,3-dihydro-1H-benzoxazol-7-yl)-piperazin-1-yl]-butyl}-amide
6-Methoxy-2H-chromene-3-carboxylic acid {4-[4-(3-methylamino-carbonyl)-piperazin-1-yl]-butyl}-amide
6-Methoxy-2H-chromene-3-carboxylic acid {4-[4-(3-mesylamino-phenyl)-piperazin-1-yl]-butyl}-amide
6-Chloro-2H-chromene-3-carboxylic acid {4-[4-(2,4-dichloro-phenyl)-piperazin-1-yl]-butyl}-amide
6-Chloro-2H-chromene-3-carboxylic acid {4-[4-(3-nitro-phenyl)piperazin-1-yl]-butyl}-amide
6-Chloro-2H-chromene-3-carboxylic acid {4-[4-(3-amino-phenyl)-piperazin-1-yl]-butyl}-amide
6 Chloro-2H-chromene-3-carboxylic acid {4-[4-(3-acetylamino-phenyl)-piperazin-1-yl]-butyl}-amide
6-Chloro-2H-chromene-3-carboxylic acid {4-[4-(3-hydroxymethyl-phenyl)-piperazin-1-yl]-butyl}-amide
6-Chloro-2H-chromene-3-carboxylic acid {4-[4-(3-mesylamino-phenyl)-piperazin-1-yl]-butyl}-amide
6-Fluoro-2H-chromene-3-carboxylic acid {4-[4-(2,3-dichloro-phenyl)-piperazin-1-yl]-butyl}-amide
6-Fluoro-2H-chromene-3-carboxylic acid {4-[4-(2-methoxy-phenyl)-piperazin-1-yl]-butyl}-amide
6-Fluoro-2H-chromene-3-carboxylic acid {4-[4-(3-cyano-phenyl)-piperazin-1-yl]-butyl}-amide
6-Fluoro 2H-chromene-3-carboxylic acid {4-[4-(3-acetylamino-phenyl)-piperazin-1-yl]-butyl}-amide
6-Fluoro-2H-chromene-3-carboxylic acid {4-[4-(3-hydroxyphenyl)piperazin-1-yl]-butyl}-amide
6-Fluoro-2H-chromene-3-carboxylic acid {4-[4-(3-nitro-phenyl)-piperazin-1-yl]butyl}-amide
6-Fluoro 2H-chromene-3-carboxylic acid {4-[4-(3-mesylamino-phenyl)-piperazin-1-yl]-butyl}-amide
6-Fluoro-2H-chromene-3-carboxylic acid {4-[4-(3-amino-phenyl)piperazin-1-yl]-butyl}-amide
6-Fluoro-2H-chromene-3-carboxylic acid {4-[4-(3-methylcarbamoyl-phenyl)-piperazin-1-yl]-butyl}-amide
6-Fluoro-2H-chromene-3-carboxylic acid {4-[4-(2,3-benzo-1,4-dioxanyl)-piperazin-1-yl]-butyl}-amide
6-Fluoro-2H-chromene-3-carboxylic acid {4-[4-(2-oxo-2,3-dihydro-1H-benzimidazol-4-yl)-piperazin-1-yl]-butyl}-amide
6-Fluoro-2H-chromene-3-carboxylic acid {4-[4-(3H-benzimidazol-4-yl)-piperazin-1-yl]-butyl}-amide
6-Fluoro-2H-chromene-3-carboxylic acid {4-[4-(2-oxo-2,3-dihydro-1H-benzoxazol-7-yl)-piperazin-1-yl]-butyl}-amide
6-Fluoro-2H-chromene-3-carboxylic acid {4-[4-(3-methyloxy carbonyl)-piperazin-1-yl]-butyl}-amide
6-Fluoro-5-(4-{4-[2H-chromene-3-carbonyl)-amino]-butyl}-piperazin-1-yl)-benzofuran-2-carboxylic acid methyl ester
2H-chromene-3-carboxylic acid {4-[4-(3,4,5-trimethoxy-phenyl)-piperazin-1-yl]-butyl})amide
2H-chromene-3-carboxylic acid {4-[4-(1H-indol-4-yl)-piperazin-1-yl]-butyl}-amide
2H-chromene-3-carboxylic acid {4-[4-(2,3-dihydrobenzo[1,4]dioxin-6-yl)-piperazin-1-yl]-butyl}-amide
2H-chromene-3-carboxylic acid {4-[4-(2,3-dihydro-benzo[1,4]dioxin-5-yl)-piperazin-1-yl]-butyl}-amide
5-(4-{4-[2H chromene-3-carbonyl)-amino]-butyl}-piperazin-1-yl)-benzofuran-2-carboxylic acid methyl ester
2H-chromene-3-carboxylic acid {4-[4-(2,3-dihydro-1H-indol-4-yl)-piperazin-1-yl]-butyl}-amide
2H-chromene-3-carboxylic acid {4-[4-(3-mesylamino-phenyl)piperazin-1-yl]-butyl}-amide
2H-chromene-3-carboxylic acid {4-[4-(1-acetyl-2,3-dihydro-1H-indol-4-yl)-piperazin-1-yl]-butyl}-amide
2H-chromene 3-carboxylic acid {4-[4-(2-oxo-2,3-dihydro-benzoxazol-7-yl)-piperazin-1-yl]-butyl}-amide
2H-chromene-3-carboxylic acid {4-[4-(2-oxo-2,3-dihydro-1H-benzimidazol-1-yl)-piperazin 1-yl]-butyl}-amide
2H-chromene-3-carboxylic acid {4-[4-(3H-benzimidazol-4-yl)-piperazin-1-yl]-butyl}-amide
2H-chromene-3-carboxylic acid {4-[4-(3-carbamoyl-phenyl-1-piperazin 1-yl]-butyl}-amide
2H-chromene-3-carboxylic acid {4-[4-(3-methylcarbamoyl-phenyl)-piperazin-1-yl]-butyl}-amide
2H-chromene-3-carboxylic acid {4-[4-(2,3-dihydro-benzofuran-7-yl)-piperazin-1-yl]-butyl}-amide
2H-thiochromene-3-carboxylic acid {4-[4-(2,3-dimethyl)-piperazin-1-yl]-butyl}-amide
2H-thiochromene-3-carboxylic acid {4-[4-(3-methyl-phenyl)-piperazin-1-yl]-butyl}-amide
2H-thiochromene-3-carboxylic acid {4-[4-(4-chloro-phenyl)piperazin-1-yl]-butyl}-amide
2H-thiochromene-3-carboxylic acid {4-[4-(2,4-dimethoxy-phenyl)piperazin-1-yl]-butyl}-amide
2H-thiochromene-3-carboxylic acid {4-[4-(3-formyl-phenyl)-piperazin-1-yl]-butyl}-amide
2H-thiochromene-3-carboxylic acid {4-[4-(3-mesylamino-phenyl)-piperazin-1-yl]-butyl}-amide
2H-thiochromene-3-carboxylic acid {4-[4-(3-nitro-phenyl)-piperazin-1-yl]-butyl}-amide
5-(4-{f-[2H-thiochromene-3-carbonyl)-amino]-butyl}-piperazin-1-yl)-benzofuran-2-carboxylic acid methyl ester
2H-thiochromene-3-carboxylic acid {4-[4-(2-oxo-2,3-dihydro-1H-benzimidazol-4-yl)-piperazin-1-yl]-butyl}-amide
2H-thiochromene-3-carboxylic acid {4-[4-(3H-benzimidazolyl)-piperazin-1-yl]-butyl}-amide
2H-thiochromene-3-carboxylic acid {4-[4-(2-oxo-2,3-dihydro-benzoxazol-7-yl)-piperazin-1-yl]-butyl}-amide
2H-thiochromene-3-carboxylic acid {4-[4-(3-methylcarbamoyl-phenyl)-piperazin-1-yl]-butyl}-amide
2H-thiochromene-3-carboxylic acid {4-[4-(3-carbamoyl-phenyl)-piperazin-1-yl]-butyl}-amide
2H-thiochromene-3-carboxylic acid {4-[4-(2,3-dihydro-benzo[1,4]dioxin-6-yl)-piperazin-1-yl]-butyl}-amide
2H-thiochromene-3-carboxylic acid {4-[4-(3-acetylamino-phenyl)piperazin-1-yl]-butyl}-amide
2H-thiochromene-3-carboxylic acid {4-[4-(2,3-dihydro-benzo[1,4]dioxin-5-yl)-piperazin-1-yl]-butyl}-amide
6-Chloro-2H-thiochromene-3-carboxylic acid {4-[4-(2,3-dihydro-benzo[1,4]dioxin 6-yl)-piperazin-1-yl]-butyl}-amide
6-Chloro-2H-thiochromene-3-carboxylic acid {4-[4-(3-cyano-phenyl)-piperazin-1-yl]-butyl}-amide
6-Chloro-2H-thiochromene-3-carboxylic acid {4-[4-(3-chloro-phenyl)-piperazin-1-yl]-butyl}-amide
61-Chloro-2H-thiochromene-3-carboxylic acid {4-[4-(3 hydroxy-phenyl)-piperazin-1-yl]-butyl}-amide
6-Chloro-2H-thiochromene-3-carboxylic acid {4-[4-(2-methoxy phenyl)-piperazin-1-yl]-butyl}-amide
6-Chloro-2H-thiochromene-3-carboxylic acid {4-[4-(2-fluoro-phenyl)-piperazin-1-yl]-butyl}-amide
6-Chloro-2H-thiochromene-3-carboxylic acid {4-[4-(2,4-dimethoxy-phenyl)-piperazin-1-yl]-butyl}-amide
6-Methoxy-2H-thiochromene-3-carboxylic acid {4-[4-(3-mesylamino-phenyl)-piperazin-1-yl]-butyl}-amide
6-Fluoro-2H-thiochromene-3-carboxylic acid {4-[4-(3-mesylamino-phenyl)-piperazin-1-yl]-butyl}-amide
6-Chloro-2H-thiochromene-3-carboxylic acid {4-[4-(3-mesylamino-phenyl)-piperazin-1-yl]-butyl}-amide

3. A method for preparing compounds of general formula 1 according to claims 1 or 2, wherein one prepares an optionally-substituted chromene or thiochromene acid of formula 2, 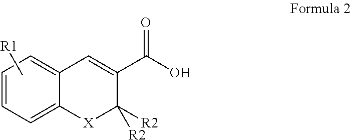
or the corresponding acid chloride, which is then coupled with a primary amine of formula 5, 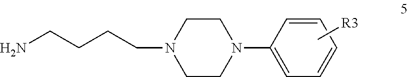
formulas wherein the various radicals R1, R2, R3, and X are defined in methylene chloride in the presence of TBTU and triethylamine, or wherein one prepares an optionally-substituted chromene or thiochromene acid of formula 2, 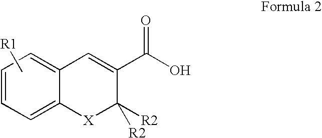
or the corresponding acid chloride, which is then coupled with 4-aminobutanol and then, after activation, an alkylation reaction is carried out with a substituted phenylpiperazine of formula 10, 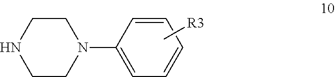
formulas wherein the various radicals R1, R2, R3, and X are defined as in claim 1.

4. A pharmaceutical composition comprising at least one compound according to claims 1 or 2, in combination with a pharmaceutically acceptable excipient.

5. (canceled)

6. Method for the treatment of a neurological or psychiatric disease or disorder comprising the administration of an effective amount of a compound according to claims 1 or 2 to a patient in need thereof.

7. The method according to claim 6, wherein the disease is Parkinson's disease.

8. The method according to claim 6, wherein the disorder is associated with a Parkinson's disease treatment.

9. The method according to claim 6, wherein the disease is psychosis.

10. The method according to claim 9, wherein the psychosis is schizophrenia.

11. Method for the treatment of dependency or addiction to drugs or other addictive substances comprising the administration of an effective amount of a compound according to claims 1 or 2 to a patient in need thereof.

12. The method according to claim 11, wherein the dependency or addiction is to nicotine or alcohol.

13. The method according to claim 6, wherein the disorder is a cognitive deficit.

14. The method according to claim 13, wherein the cognitive deficit is associated with Alzheimer's disease or is related to aging.

15. The method according to claim 6, wherein the disorder is depression.

16. The method according to claim 6, wherein the disorder is essential tremor.

17. The compound according to claim 1, wherein the ring fused formed by R3 with the aromatic ring which carries it, is an aryl, heteroaryl or C₅, C₆ or C₇ cycloalkyl or a heterocycle.
